(19) United States
(12) Patent Application Publication
(10) Pub. No.: US 2016/0278366 A1
(43) Pub. Date: Sep. 29, 2016

(54) Microfluidic embryo and gamete culture systems

(71) Applicant: H. Randall Craig, Phoenix, AZ (US)

(72) Inventor: H. Randall Craig, Phoenix, AZ (US)

(21) Appl. No.: 15/164,783

(22) Filed: May 25, 2016

Related U.S. Application Data
(62) Division of application No. 13/645,495, filed on Oct. 4, 2012.
Application No. 13/645,495 is a division of application No. 13/062,515, filed on Mar. 7, 2011, now abandoned.
(60) Provisional application No. 61/114,365, filed on Nov. 13, 2008.
(60) Provisional application No. 61/113,581, filed on Nov. 11, 2008.

(51) Int. Cl. A01N 1/02; A61B 17/435

(52) U.S. Cl. CPC A01N 1/0284; A61B 17/435; A01N 1/0252

(57) ABSTRACT

A robotic microfluidic incubator system has a thin transparent sidewall and close proximity of the embryo/oocyte/cultured cells to the sidewall allow close approach of a side view microscope with adequate focal length for mid to high power. This arrangement permits microscopic examination of multiple culture wells when arranged in rows (linear or along the circumference of a carousel). Manual or automated side to side movement of the linear well row, or rotation of the carousel, allows rapid inspection of the contents each well. Automated systems with video capability also allow remote inspection of wells by video connection or Internet connection, and automated video systems can record oft-hours inspections or time lapse development in culture (i.e. embryo cell division progression, or axon growth in neuron cell cultures).

### CROSS REFERENCE TO RELATED APPLICATIONS

This application claims the benefit of U.S. Provisional Application No. 61/113,581, filed Nov. 11, 2008, and U.S. Provisional Application No. 61/114,365, filed Nov. 12, 2008, each of which are incorporated herein by reference for all purposes.

### BACKGROUND OF THE INVENTION

Classic cell culture consists of cells and tissues grown in Petri dishes containing large amounts of culture media and stored in large temperature and humidity controlled incubators. Microfluidic cell culture systems enclose cells and tissue specimens in tiny fluid-filled chambers and channels, reducing the scale of biologic culture systems in the same manner that integrated circuits reduced the scale of electronics from vacuum tubes and transistors.

Significant advantages of microfluidic culture systems include small laboratory size, reduced laboratory expenditures, automated cell culture media changes and manipulations, and numerous labor saving innovations. Purification of sperm from semen and separation of normal sperm from those with chromosomal and morphologic abnormalities, and the potential to separate X and Y chromosome sperm, is a goal of the sperm separation network. The vertical micromanipulator system simplifies manual and automated manipulation of cells, gametes, and neurons for research and clinical applications. The microfluidic cell culture cassette system allows massive parallel system advantages for cell and tissue cultures, and provides flexibility in initiating, storing, and moving cell cultures between specific applications. The rapid culture media/multiple gas equilibrator eliminates the one to two hour dissolved gas equilibration time characterized by current incubation systems, allowing immediate availability of small volumes of feedback controlled pre-equilibrated media supplied to microfluidic cell culture systems. Intra-vaginal incubation modules eliminate large, expensive cell culture incubators and the associated multiple gas lines and manifolds, with the added benefit of providing in-vitro fertilization (IVF) patients a more intimate role in their fertility treatment. The biospecimen microfluidic freezing stem should significantly improve freeze-thaw survival of cells and tissues while protecting the specimens from microorganism contamination during cryopreservation.

Current IVF technology can involve up to eight steps:

1. sperm purification;

2. oocyte capture and isolation;

3. oocyte stripping;

4. intra cytoplasmic sperm injection (ICSI);

5. embryo incubation;

6. embryo & oocyte freezing;

7. embryo & oocyte thawing; and

8. zona hatching and embryo transfer.

### Current Sperm Purification and Separation Methods

Purification of sperm from semen, washing away cellular debris, and reconcentration of sperm is an essential requirement for many fertility procedures, including preparation of sperm for intrauterine insemination and for IVF. Separation of normal sperm from those with chromosomal and morphological abnormalities is difficult with current technology.

Purified sperm are used primarily for intrauterine insemination or as the initial preparation for IVF or ICSI. Currently used methods for sperm purification include basic sperm wash with resuspension in low volume media, sperm swim up procedure from centrifuge pellet into low volume media, density gradient purification with one or two density layers, or transverse of sperm through bovine mucus filter.

Separated sperm are used primarily for IVF, gender selection, or pre-implantation genetic diagnosis procedures. Currently used technology with relatively low efficiency for separation of sperm includes filtering sperm through a concentrated albumin solution, subjecting sperm to column chromatography, or layering sperm on a density gradient solution and applying high centrifugation forces. A much more efficient but expensive method for separation of sperm utilizes flow cytometry to individually select and separate sperm based on optical properties.

### Oocyte Capture and Isolation

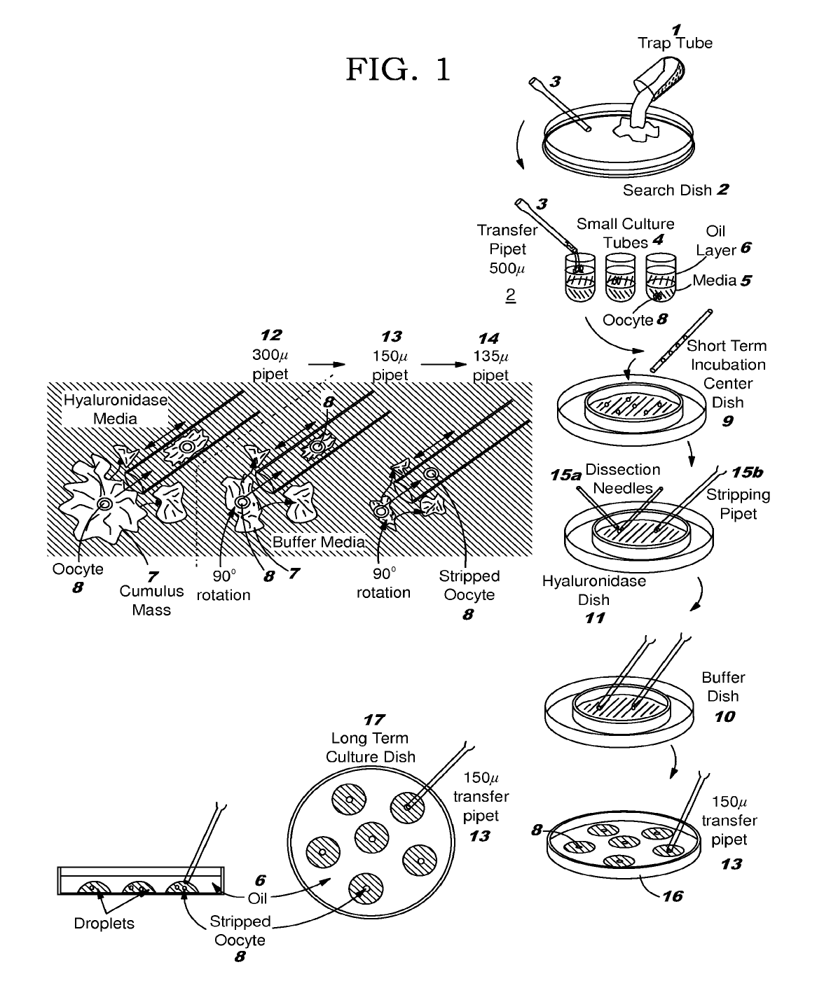

Currently, five steps are performed sequentially to capture and isolate oocytes, which are illustrated in FIG. 1:

1. Oocytes are aspirated from ovarian follicle using 17-gauge needle under ultrasound guidance and vacuum pump, using 10-ml plastic tube fluid trap 1. The trapped fluid tube 1 is then detached and passed to an IVF lab technician and placed in heating block.

2. The trap tube 1 is emptied into a search dish 2 and examined under stereo microscope. Cumulus masses 7 containing oocytes 8, along with bare oocytes 8, are identified and then aspirated into 500 μm roller-controlled pipettes 3 and transferred into individual 5 milliliter test culture tubes 4, each containing 1.0 cc of HEPES media 5 under 0.7 cc mineral oil 6 which has been pre-equilibrated in an incubator. Cumulus masses 7 are deposited on top of the oil layer and spontaneously sink through the oil layer into media, separating from red blood cells and cell debris during the oil passage. One, or occasionally two, oocytes 8 are inserted in each tube 4, with tubes 4 kept in heating block until the oocyte capture procedure is completed. The tubes are capped before and after receiving oocytes to maintain dissolved gas equilibrium.

3. The heating block containing the small culture tubes is moved to the IVF lab and placed in a laminar flow hood. Preincubated and equilibrated center culture dishes 9 containing 1.0 cc of buffered culture media under 0.7 cc oil are then moved from incubator to hood, and placed under stereo microscope.

4. The small culture tubes 4 are uncapped and the cumulus masses 7 are aspirated into a 500 μm pipette, up to 4 to 6 oocytes into the pipette at a time. Oocytes and cumulus masses are then transferred to center culture dishes 9 through the oil layer, usually 4 to 12 per dish. The pipette is used to evenly distribute the cumulus masses on dish bottom. The center culture dishes 9 are lidded and transferred to an incubator for 1½ to two hours. Incubator settings are temperature of 37.0° C., CO₂ gas of 5.8%, and oxygen gas of 18.9%.

### Oocyte Stripping Procedure

1. Preincubated and equilibrated center culture dishes are moved to hood within 30 minutes after egg capture procedure, two dishes 10, 16 contain 1.0 cc of hepes media and one dish 11 contains 1.0 cc hyaluronidace media. The dish 11 is placed under sterile microscope. The oocyte center dishes 9 are then moved to hood and the lids are removed.

2. Three to four cumulus masses are transferred from the oocyte center dish 9 to the hyaluronidace dish 11 and incubated for 45 to 60 seconds.

3. Oocytes 8 are then individually aspirated into 300 μm roller pipette 12, then pulled back and forth, to and fro, passing repeatedly through the pipette mouth with the outer layer of cumulus mass peeled off. Pipette stripping usually requires 5 to 15 rapid passes. Oocytes 8 are then returned to the bottom of the hyaluronidace dish 11 and the pipette stripping procedure is then repeated on the next oocyte.

4. The stripping procedure is rapidly repeated on the same oocytes using a 150 μm pipette 13, with 90 degree rotation of oocytes 8 done to facilitate removal of remaining cumulus 7. Once mostly stripped, oocytes 8 are collected into the 150 μm pipette 13 as a group and transferred out of the hyaluronidase dish 11 and into the first buffered dish 10. The next set of 3 to 4 oocytes in center dish are selected and the stripping procedure is repeated.

5. The oocytes 8 in the first buffered dish 10 are then stripped of any remaining cumulus 7 using the smaller 135 μm pipette 14, then transferred to second buffered dish 16. Oocytes 8 with very tight or adherent cumulus are manually dissected with two 27 gauge metal needles 15a, 15b chopsticks style.

6. After stripping, the oocytes 8 are transferred to the long-term IVF culture dish 17 using the 150 μm pipette 13. The long-term culture dishes 17 are then placed in the incubator until fertilization or ICSI procedure.

### ICSI—Intra-Cytoplasmic Sperm Injection

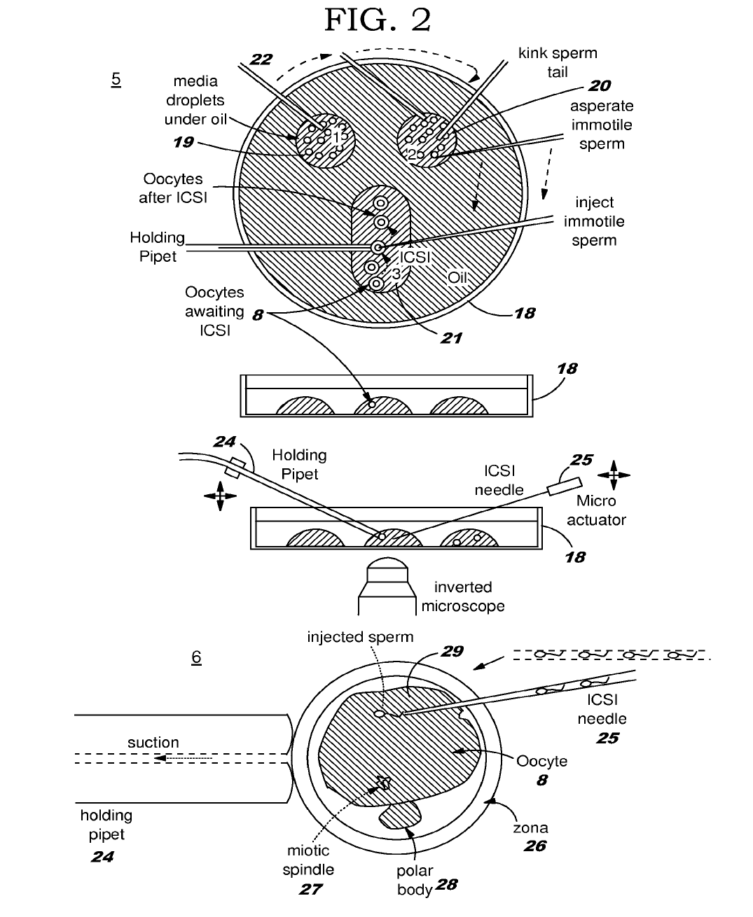

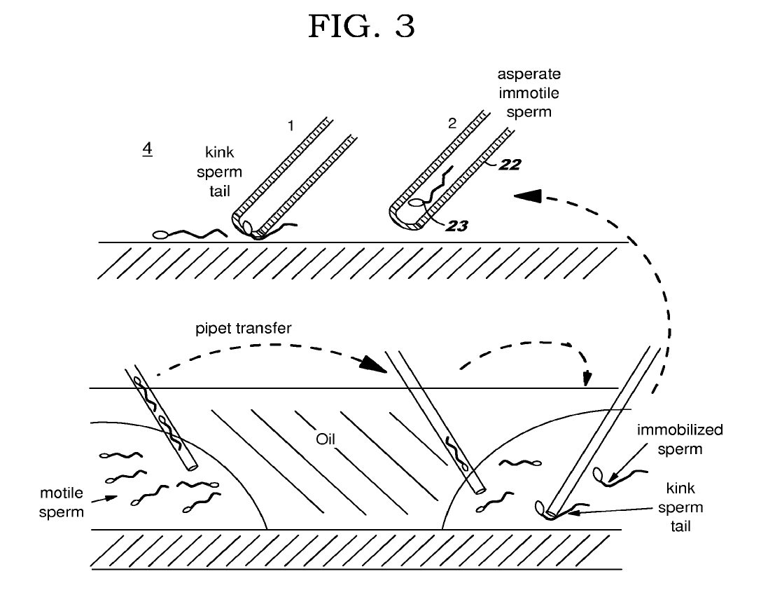

ICSI is illustrated in FIGS. 2 and 3.

1. ICSI dish is prepared in a 10-cm Petri dish 18 by placement of two round drops 19, 20 and one elongated drop 21 evenly spaced in the dish 18. The upper left drop 19 is 0.5 cc HTF plus 10% SPS solution, the upper right drop 20 is 0.5 cc of the same solution but containing PVP (polyvinylpyrrolidone) which is required to clean sperm and slow sperm velocity. The lower middle elongated drop 21 contains 1.0 cc of hepes buffer solution. All 3 droplets are kept under oil and pre-equilibrated in the incubator for two hours.

2. The processed sperm solution is examined in its test tube, and a 250 μm pipette 22 is used to transfer several thousand sperm into the upper left droplet 19 in the ICSI dish 18. A few dozen sperm that progressed rapidly to the opposite edge of the droplet are collected in the same pipette 22 and transferred to the upper right PVP droplet 20. The long-term IVF culture dish 17 is removed from the incubator and placed next to the ICSI dish 18, and between 1 to 5 stripped oocytes 8 are aspirated into the 250 μm pipette 22 and then transferred to the lower end of the elongated droplet 21. They are aligned adjacent to each other in a vertical row.

3. The 250 μm pipette 22 is then used to individually trap the morphologically best appearing sperm 23 against the bottom of the Petri dish in the PVP droplet 20, with the sperm 23 held one third the distance down the tail from the head position. The sperm 23 tail at this point is then kinked with the pipette to immobilize the sperm. After this has been completed for 4 to 5 sperm 23, the immobilized sperm are then transferred with the same pipette 22 to the middle of the elongated droplet 21.

4. The 80 μm diameter holding pipette 24 is then inserted into the left actuator of the micromanipulator, and the 10 μm diameter microneedle 25 is inserted into the right actuator of the micromanipulator. They are then lowered into the middle of the elongated droplet 21 under microscopic guidance. The holding pipette 24 is then used to approach the uppermost oocyte 8, suction is applied to grasp the oocyte 8 at the end of the holding pipette 24, and the pipette 24 is then moved back to the middle of the elongated droplet 21.

5. The 10 μm microneedle 25 is then used to aspirate 4 or 5 sperm 23 along its length with the head of the sperm 23 oriented toward the tip of the needle 25. Using alternating flush and suction through the holding pipette 24, the oocyte 8 is rotated and oriented until the polar body 28 is at the 6 o'clock position. The microneedle 25 containing sperm 23 is then used to puncture the zona 26 followed by the oocyte 8 membrane at the two o'clock position in a horizontal direction to avoid the miotic spindle 27. The first sperm 23 at the tip of the microneedle 25 is slowly injected into the cytoplasm 29 as the microneedle 25 is gradually withdrawn. After inspection, the injected oocyte 8 it is then moved to the top of the elongated droplet 21 and released. The holding pipette 24 is then moved back to the bottom of the elongated droplet 21 to grasp the next oocyte 8. Slow injection of fluid out of the microneedle 25 is done until the next sperm 23 is positioned at the tip of the needle 25.

6. This procedure is repeated for each sequential oocyte 8 until sperm injection has been performed on all, taking care to inject as little media as possible into the cytoplasm 29 during sperm injection. After completing the procedure, all ICSI fertilized oocytes 8 are located at the top of the elongated droplet, and they are then aspirated en mass into the 250 μm pipette 22 and transferred back into the long-term IVF culture dish 17. The culture dish 17 is then returned to the incubator.

### Incubation (No Figures)

1. Preincubated and equilibrated long-term culture dishes are moved to hood. Each stripped oocyte in second buffer dish is individually transferred to its own culture media droplet under oil in the long-term culture dish, using a 150 μm micropipette inserted directly into the droplet. The long-term dishes are then moved back into the incubator.

2. Incubator settings are temperature at 37° C., oxygen at 18.9%, and carbon dioxide at 5.8%.

3. Incubator atmosphere consists of controlled concentrations of oxygen, nitrogen, and carbon dioxide provided by a programmed gas mixing manifold which is supplied by three gas lines from individual compressed gas tanks cylinders.

4. Embryos observed each day to evaluate progress and development. Embryo culture dishes are removed from incubator and viewed under the inverted microscope, then quickly returned to the incubator. Expected progress on day 1 after ICSI is confirmation of fertilization by presence of two pronucleii and/or a second polar body. Day 2 embryos should be at the 4 cell stage, day 3 embryos at the 8 to 12 cell stage, and day 4 embryos at the compact morula stage. If embryo culture is continued to day 5, blastocyst development should be expected.

5. Cell culture media fluid is changed from HTF on the first day of culture to pyruvate based media, which is continued until day 4. It is then changed to glucose based media which is continued until termination of culture.

6. Embryos are incubated until embryo transfer procedure, embryo freezing, or are discarded if development stalls of fails. Depending upon the in vitro fertilization program, embryos are transferred or frozen typically on day 1, day 3, day 4, or day 5 after egg capture and ICSI.

### Embryo Freezing (Cryopreservation)

1. Cryopreservation solutions are mixed prior to cryopreservation procedure, then stored in lab refrigerator at 4° C. until freezing procedure. Cryopreservatives are propylenediol and sucrose, in hepes buffer solution at three increasing concentration levels. Embryos are immersed in each solution sequentially from lowest to highest concentration for specific time periods, allowing time to establish osmotic equilibrium in each solution before transfer to the next. After spending a short period of time incubating in the highest concentration solution, the embryos are transferred to a freezing vial containing the same solution and then inserted into a programmable freezing machine. Once frozen, the vials are removed from the machine and stored in a liquid nitrogen cryostat until thawing procedure.

2. Cryopreservation solution dish is prepared by placing 0.5 cc droplets of all 4 solution levels in each quadrant of a 5-cm Petri dish, all under oil layer, with each droplet labeled “0” to “3” with marker pen on the dish underside. Typically, two additional buffered drops without cryopreservation (level 0) are added as back-up rinse droplets. This dish is temperature and gas equilibrated, but all prior prep steps are subsequently done at room temperature in open hood.

3. Freezing vials are prepped by pipetting highest concentration solution (level 3) into each vial (0.5 cc each) in open hood. Vials are pre-labeled and will contain one or two embryos each. After solution is added to each vial, the vial lids are reattached to prevent evaporation before embryos are inserted.

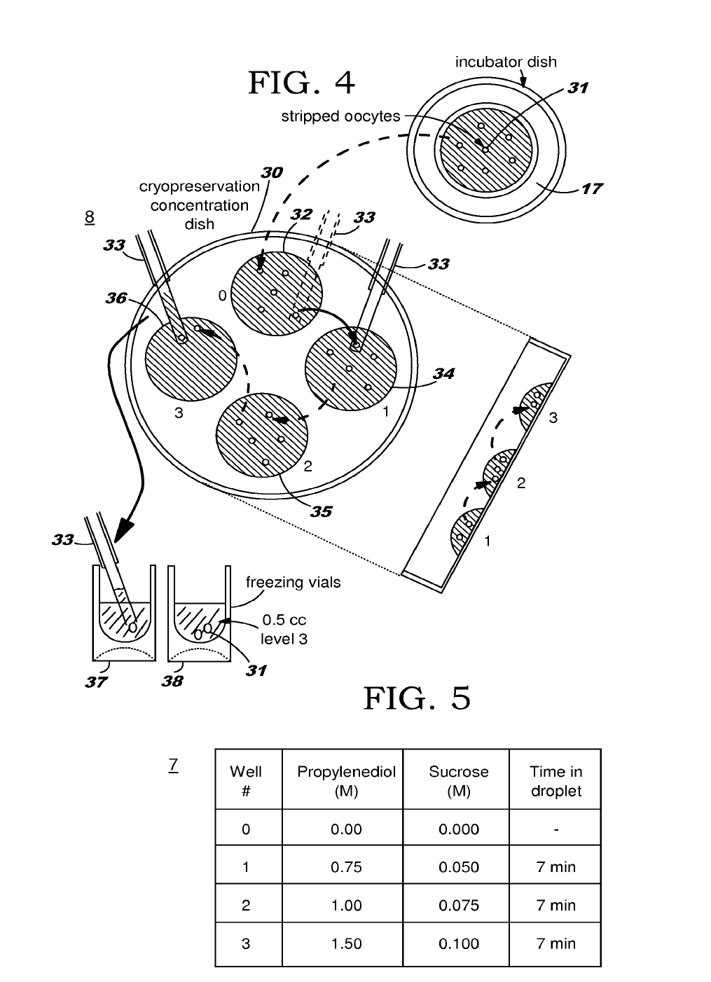

4. Referring to FIG. 4, embryo culture dishes 17 are removed from the incubator and these 10-cm dishes are placed adjacent to the cryopreservation solution dish 30 in the open hood at room temperature. The first embryos 31 are aspirated with accompanying micro-drop of culture solution into 180 μm micropipette 33 under microscopic visualization, and then transferred directly through the oil layer into the level 0 droplet 32 using the same micropipette 33, with placement of embryos 31 evenly spaced in the center of the droplet 32. Once all the embryos 31 are in place in the level 0 droplet 32, they are individually reaspirated into the same micropipette 33 and transferred through the oil layer directly into the level 1 droplet 34 under microscopic visualization. Embryos 31 are incubated in the level 1 droplet 34 for 7 minutes by electronic timer. The embryos 31 are then reaspirated into the micropipette 33 and transferred to the level 2 droplet 35 and incubated in that solution for 7 minutes. Finally, the embryos 31 are transferred to the level 3 solution 36 for an additional 7 minutes using the same method. Composition of level 1, level 2 and level 3 solutions is shown in FIG. 5.

5. The embryos are then immediately transferred by the 180 μm micropipette 33 into the freezing vials 37, 38, one or two embryos 31 per vial 37, 38. The vials 37, 38 are sealed by screw on caps, then loaded into the programmable freezer.

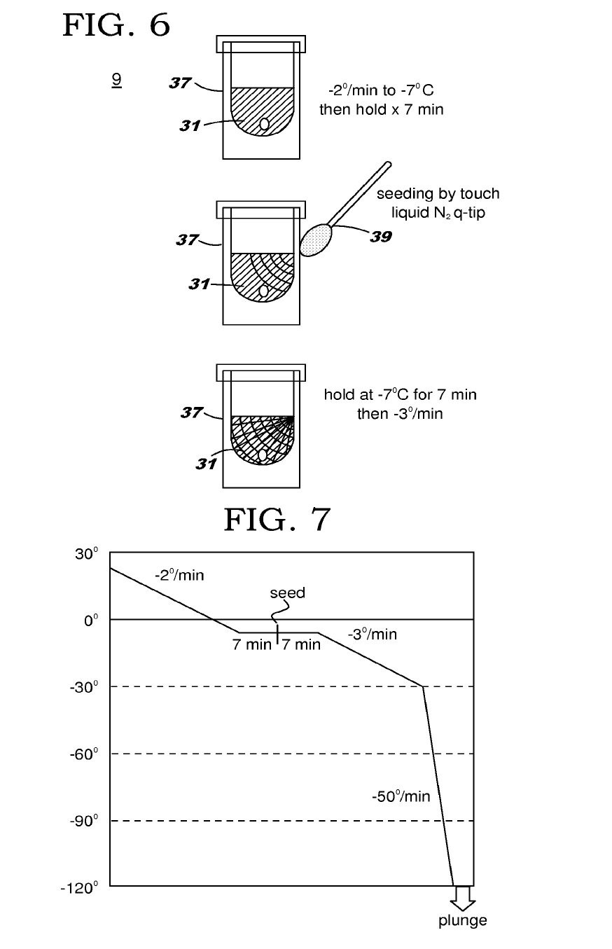

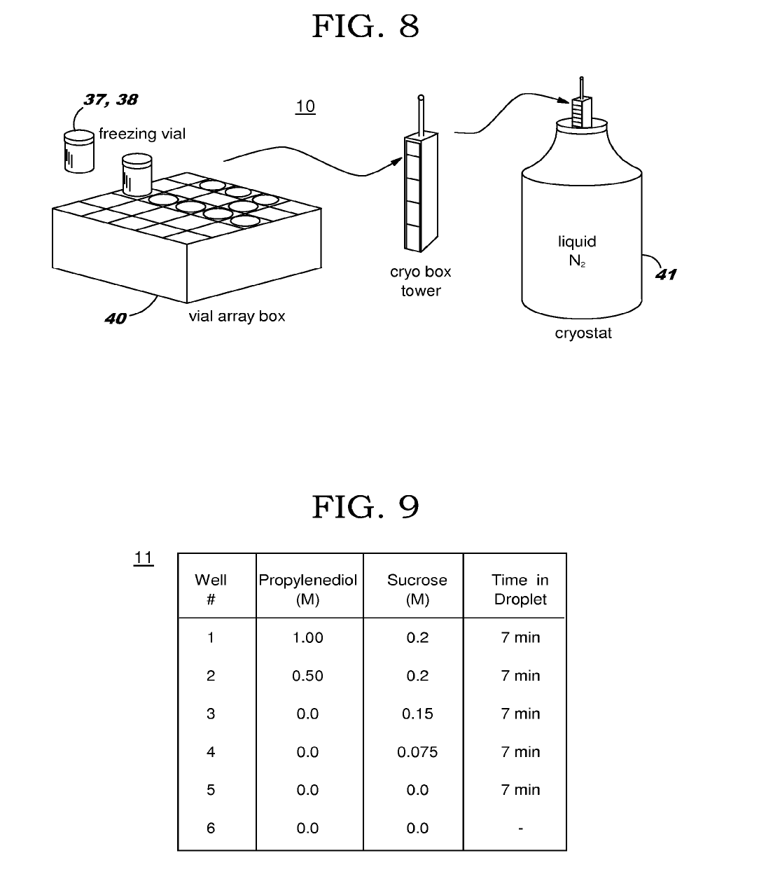

6. In the freezer, embryos 31 are initially cooled at 2° C./min down to −7° C., then held at −7° C. for 5 minutes. The vials 37, 38 (only one is shown) are then individually seeded by placing a Q-tip presoaked in liquid nitrogen 39 briefly on the outside wall of the vials 37, 38 just at the media solution level to start ice crystallization of the super cooled media from the surface down toward the bottom of vial. Embryo vials 37, 38 are held at −7° C. for additional 7 minutes, then cooled at minus 3° C./min to a temperature of −30° C., the cooling rate is increased to −50° C./min to a temperature of −120° C. See FIG. 7. Referring to FIG. 8, vials 37, 38 are then plunged into liquid nitrogen for one minute, inserted into storage cartons 40 and stored in liquid nitrogen cryostat 41.

7. Liquid nitrogen cryostat holds vials in cartons under the surface of liquid nitrogen and, with the added safety feature of cryogen level and temperature sensors activating audio and computer phone alarms. Frozen embryos and sperm can be held for decades without loss of viability. To retrieve a specific embryo, the entire stack of vial cartons in the assigned group must be pulled up and out of the cryostat, the cart removed and opened, and the vial withdrawn, and the process reversed to replace the carton stack back into the cryostat before appreciable warming can occur.

### Embryo Thawing

1. Thawing media solutions are mixed and stored in the lab refrigerator, then warmed at room temperature before the thaw procedure begins. The thawing procedure is the approximate reversal of the cryopreservation procedure, with the modification of using 5 intermediate cryopreservation concentration levels instead of 3 levels. All dilution media are hepes solution with decreasing concentrations of propylenediol and sucrose sequentially down to zero. All dilution solutions are prepared in advance in culture flasks within one week of use. FIG. 9 shows the composition of the solutions.

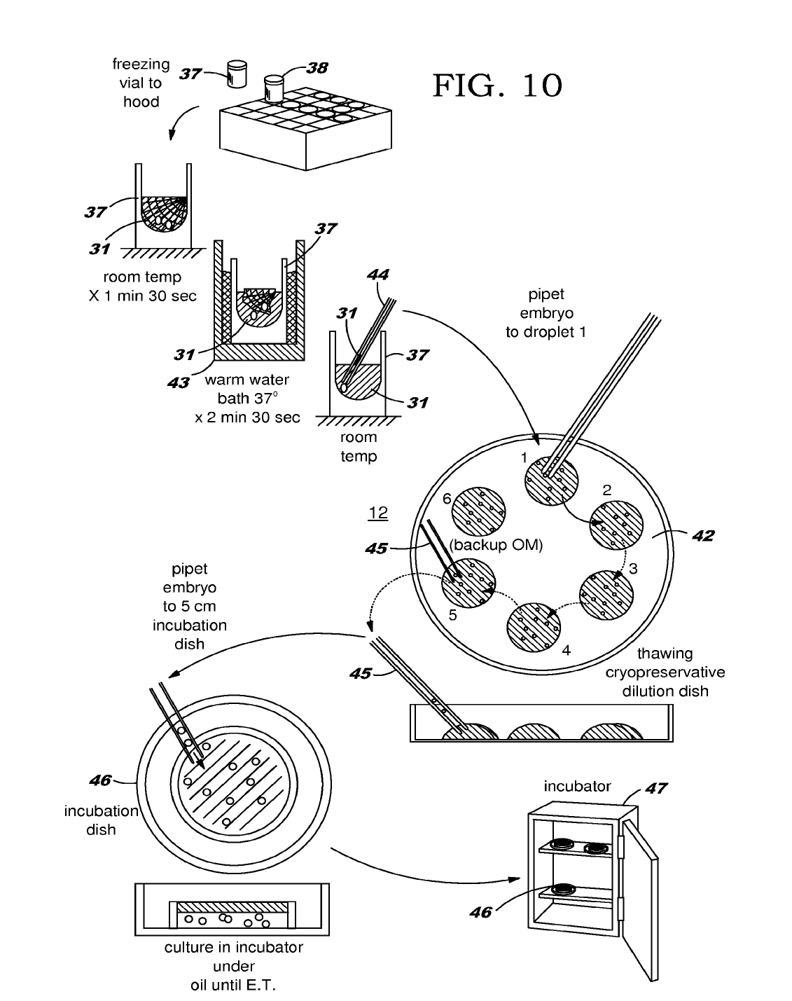

2. Referring next to FIG. 10, the dilution dish 42 is prepared by placing 0.8 cc drop of each solution in a 5-cm Petri dish, a total of 6 drops in a radial pattern. Drop 5 and 6 have no cryopreservative, with drop 6 used as a back up buffer solution for occasional final rinse. No cover oil is used and dilution is done at room temperature in laminar flow hood.

3. The appropriate frozen embryo vials 37, 38 are removed from the cryostat and placed on hood surface at room temperature for 1 minute 30 seconds, then immersed in 37° C. water bath 43 for 2 minutes 30 seconds, and placed back on hood surface at room temperature.

4. Individual embryos 31, one at a time, are aspirated from thawing vials 37 and transferred directly into drop number one using a 30 degree angle roller pipette 44 (400 μm diameter) with small volume of fluid.

5. Embryos 31 are incubated in drop number one for 7 minutes, then transferred with straight micropipette (400 μm diameter) to drop number two. Embryos are then sequentially incubated for 7 minutes in each drop (1 to 6) transferred with 180 μm pipette 45, with the Petri dish covered between transfers.

6. After the last 7 minute incubation in drop 5, the embryo is transferred to a separate 5-cm Petri dish 46 and into hepes-free media drops under oil cover using the same 180 μm straight pipette 45, then covered, and the Petri dishes 46 then moved into the incubator 47 for storage until embryo transfer procedure.

### Embryo Hatching and Transfer Procedure

1. Insert Green holding micropipette (O.D.=150 μm, I.D.=30 μm) into coupler, then coupler is inserted into left-hand side micromanipulator actuator. Holding pipette position is checked by observing through inverted microscope and lowering to staging position by the z axis knob. The red 15 degree angle hatching microneedle (3 to 4 μm diameter) is inserted into its coupler, then coupler is inserted into the right hand sided micro-actuator, then lowered into the staging position by its right z-axis knob.

2. The identification of embryos to be transferred is checked and confirmed by lab records, including incubation and dish and micro-drop numbers. The appropriate culture dish this removed from the incubator and placed under the stereo microscope. A 250 μm diameter micropipette is inserted into a thumb control suction unit handle and is then used to inspect the embryos after removing the incubation dish cover.

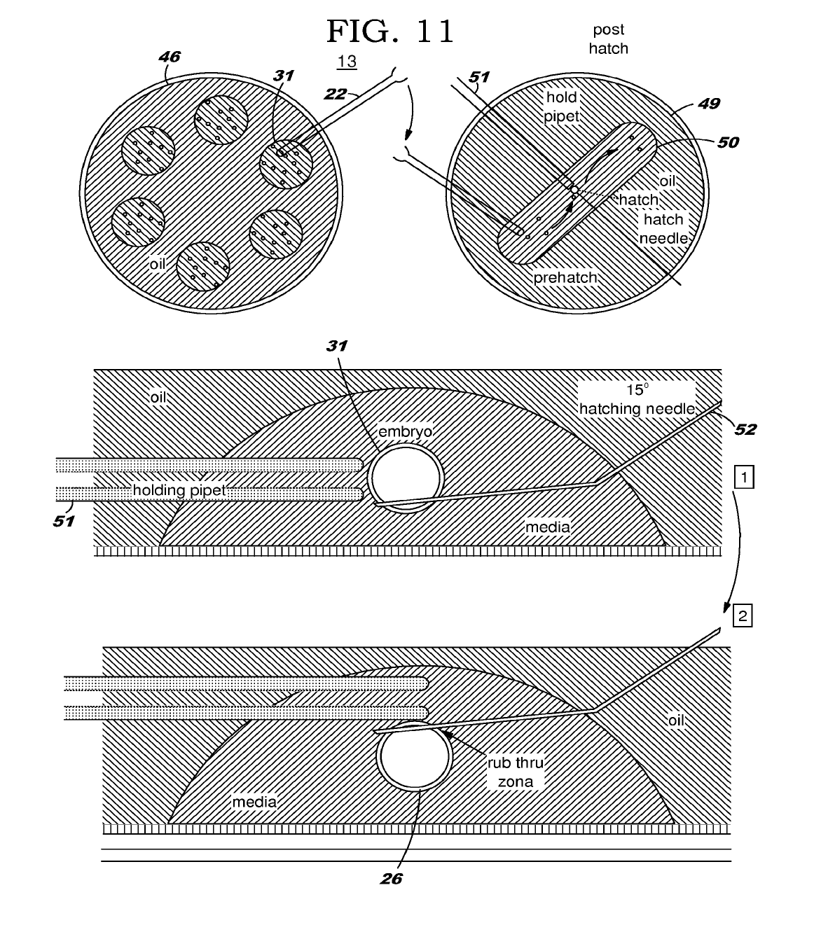

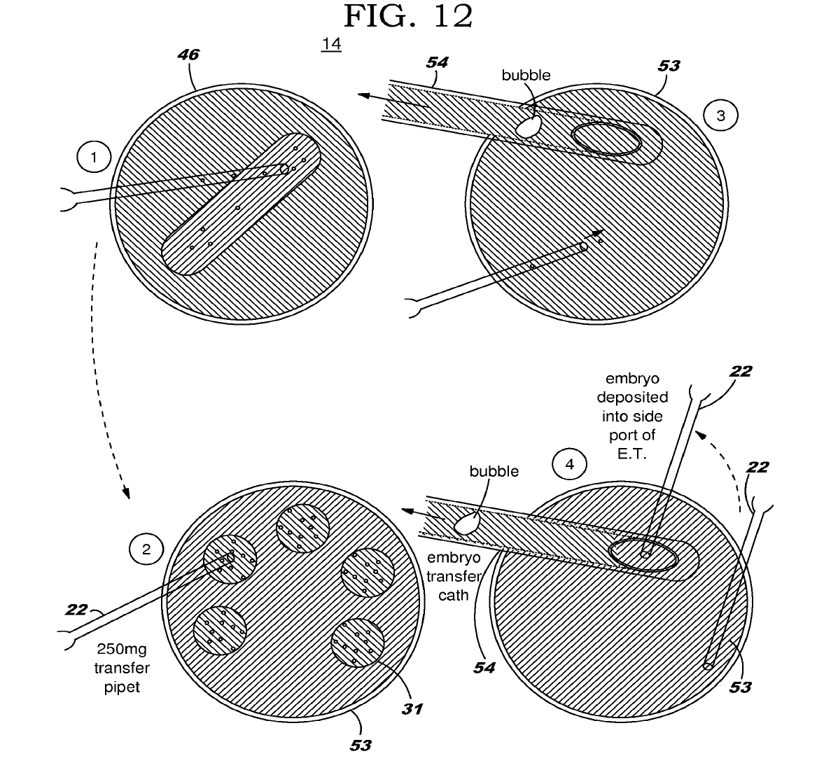

3. Referring next to FIG. 11, the 250 μm pipette 22 is used to aspirate the first embryo 31 from the patient dish 46 microdrop and then transfer the embryo into the hatching dish 49 elongated microdrop 50. The hatching dish 49 contains a droplet 50 of approximately 0.5 cc hepes buffer solution with 10% SPS, under oil cover. One to six embryos 31 are transferred individually to the lower end of the elongated drop 50. The patient dish 46 is returned temporarily to the incubator 47 and held at 37° C., 19.6% oxygen, and 5.5% carbon dioxide.

4. Hatching dish 49 is then moved to the inverted microscope stage, and the holding pipette 51 and hatching microneedle 52 are lowered into the elongated drop under 100 power magnification. The magnification is increased to 400 power and the x and y axis holding pipette 51 is manipulated to the first embryo 31, suction applied to capture it, and then manipulated to the middle of the elongated drop 50. The right x and y axis actuator is used to move the 15 degree microneedle 52 to the opposite side of the embryo 31, then penetrate the zona 26 through a shallow arc and emerge into holding pipette 51 lumen. The right actuator is then used to detach the embryo 31 from the holding pipette 51 after release of suction, and rub the zona 26 against the outer terminus of the holding pipette 51 down to the penetrating microneedle 52, cutting a slit in the zona 26 to complete the hatching procedure. The embryo 31 is moved to the upper end of the elongated drop 50, and the procedure is then repeated for all remaining embryos 31. Hatched embryos 31 are then returned to the incubator dish 46 using the 250 μm micropipette 22 under the stereo microscope at 40 power magnification.

5. When the patient is ready, the incubator dish 46 with hatched embryos 31 is removed from the incubator 47 and placed under the stereo microscope, inspected, and moved to the warming surface. The side port embryo transfer catheter is attached to a 1 cc syringe filled with buffer media which is then injected through the catheter to check for leaks.

6. Referring next to FIG. 12, hatched embryos 31 are then transferred to a 5-cm Petri dish 53 containing 10 cc of hepes media with 10% SPS, using the 250 μm micropipette 22. The embryo transfer catheter 54 is then lowered into this dish 53, side-port up, under the media surface and its syringe is pulled back to the 0.5 cc position. The embryo transfer catheter 54 is then lifted into air above the dish and a small bubble is aspirated into the side-port, and the embryo catheter 54 is returned to the media and the bubble is then aspirated 3 to 4 cm into the catheter. The hatched embryos 31 are aspirated en masse into a 250 μm pipette 22 and deposited into the side-port of the embryo transfer catheter 54, then aspirated 3 to 4 cm into the embryo transfer catheter 54.

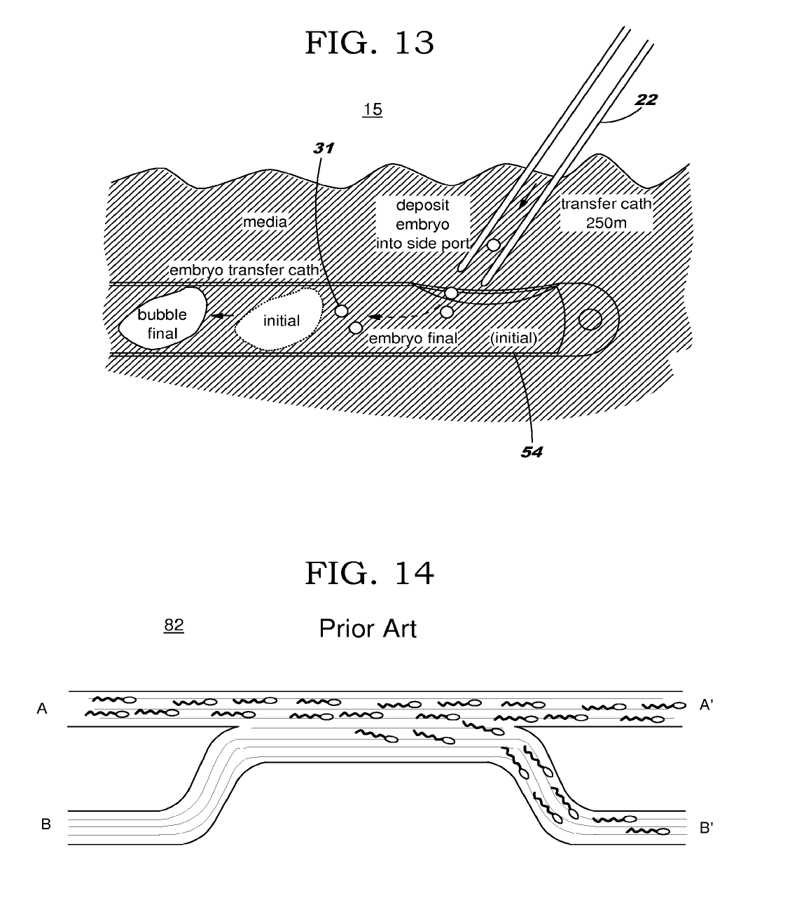

7. Referring next to FIG. 13, embryo transfer catheter 54 is then removed from the Petri dish 53 and delivered to physician for the embryo transfer procedure.

### SUMMARY OF THE INVENTION

Commercial in vitro fertilization laboratory procedures are largely characterized by sequential repetitive cell culture and micromanipulation steps currently performed by antiquated manual cell culture lab techniques. A relatively small number of standard lab manipulation and incubation steps performed in consistent sequential order makes In Vitro Fertilization (IVF) procedures especially amenable to automated microfluidic cell culture, using standard and easily programmable laboratory algorithms. Microfluidic cell culture and cell transport techniques are potentially much more effective and efficient for IVF applications than currently used standard Petri dish and cell culture-in-test tube incubators. Current IVF lab procedures involve culturing simple tiny cells (embryos, oocytes, sperm) in relatively enormous cell culture media volumes in dishes or test tubes, whereas microfluidic systems incubate cells in small micro-chambers. Why store a Volkswagen in an aircraft hanger when an automobile garage is much more efficient and practical? The microfluidic systems are also very amenable to automated micro-manipulation of cells and embryos, and may easily benefit from microprocessor control.

Microfluidic systems can perform several primary functions for IVF and embryo culture: Get sperm and oocytes together for fertilization, supply culture media and nutrients to developing embryos, and transport gametes and embryos between specialized procedures.

Microfluidic systems can prepare gametes and get sperm and oocytes together for fertilization. Such systems can process raw sperm, separating active mobile sperm from semen, cell debris, and immobile or defective sperm. Further, such systems can capacitate sperm by holding in appropriate medium or adding capacitating factors to incubating sperm. Such systems can purify sperm and separate sperm groups by specific physiologic or physical properties, i.e. by activity level or velocity, density, chemotactic differential. Further, they can transport sperm to specialized culture chambers for holding, staging, incubating, ICSI, or fertilization. They can load sperm into pipettes or catheters for intra-culture transport, intrauterine insemination, or sperm freezing containers. Such systems can strip oocytes of cumulus cells or mucus cell debris and transport oocytes to specialized culture chambers for IC SI, fertilization, etc. Finally, microfluidic systems can load oocytes into pipettes or catheters for intra-culture transport or oocyte freezing.

Further, microfluidic systems can supply culture media and nutrients to gametes and developing embryos. They can sequentially change culture media to match embryo development stage, namely HTF for sperm and oocytes, pyruvate base for multi-cell embryos, intermediate for morula stage, glucose based for blastocyst, sodium depleted for oocyte freezing, etc. Such systems can sequentially concentrate or dilute cryopreservatives and media prior to freezing or after thawing oocytes, sperm, or embryos. They can supply fresh media by slow-flow to embryos during incubation and remove waste media from culture, including free radicals. Concentrations of dissolved gases in culture media (nitrogen, oxygen, carbon dioxide) can be tightly controlled, thus eliminating the need for culture fluid/gas atmosphere interface and associated prolonged equilibrium time. Such systems can automate and simplify sampling of culture media for chemical analysis. Finally, co-culture of oocytes and embryos with other cell types, including endometrial cells and tubal lining cells can be automated and miniaturized by including separate culture chambers with shared or transferred media and/or common culture chambers for simultaneous or sequential co-culture.

Finally, microfluidic systems can transport gametes and embryos between various culture chambers. Gametes or embryos can be moved between open or closed culture chambers. Gametes or embryos can be moved between open culture chambers using a multi-well, carousel or similar system. Open chambers can be supplied with slow flow media nutrients systems described above. Gametes and embryos can be moved between open chambers by a micropipette system. A combined open and close chamber system is very versatile and allows optimal culture conditions and micromanipulation procedures in a single combined system. A microfluidic system reduces or eliminates the risk of accidental dropping or loss of culture and embryos because manual movement of culture dishes or tubes between incubators or microscope stages is no longer necessary. Movement of embryos between micro-chambers for specialized functions and procedures can be simplified, or even automated, including: preparation (sperm capacitation, oocyte stripping, cryopreservative concentration and dilution); staging (holding cells between culture and procedure chambers); micromanipulation (temporary placement of oocytes/embryos for micromanipulation procedures including ICSI, blastomere biopsy, assist hatching, etc.); and catheter or freezing chamber loading or unloading.

### BRIEF DESCRIPTION OF THE DRAWINGS

FIG. 1 shows a current method to capture and isolate oocytes.

FIG. 2 shows intra-cytoplasmic sperm injection.

FIG. 3 shows the preparation of sperm for intra-cytoplasmic sperm injection.

FIG. 4 shows a method of preparing embryos for cryopreservation.

FIG. 5 shows the composition of the solutions used for embryo cryopreservation.

FIG. 6 shows a method of embryo cryopreservation.

FIG. 7 shows the cooling rate of the embryos.

FIG. 8 shows a storage method for cryopreserved embryos.

FIG. 9 shows the composition of solutions used for thawing cryopreserved embryos.

FIG. 10 shows a method for thawing cryopreserved embryos.

FIG. 11 shows a method for hatching embryos.

FIG. 12 shows a method for transferring hatched embryos.

FIG. 13 shows hatched embryos being transferred to an embryo transfer catheter.

FIG. 14 shows fluid flow in microfluidic channels.

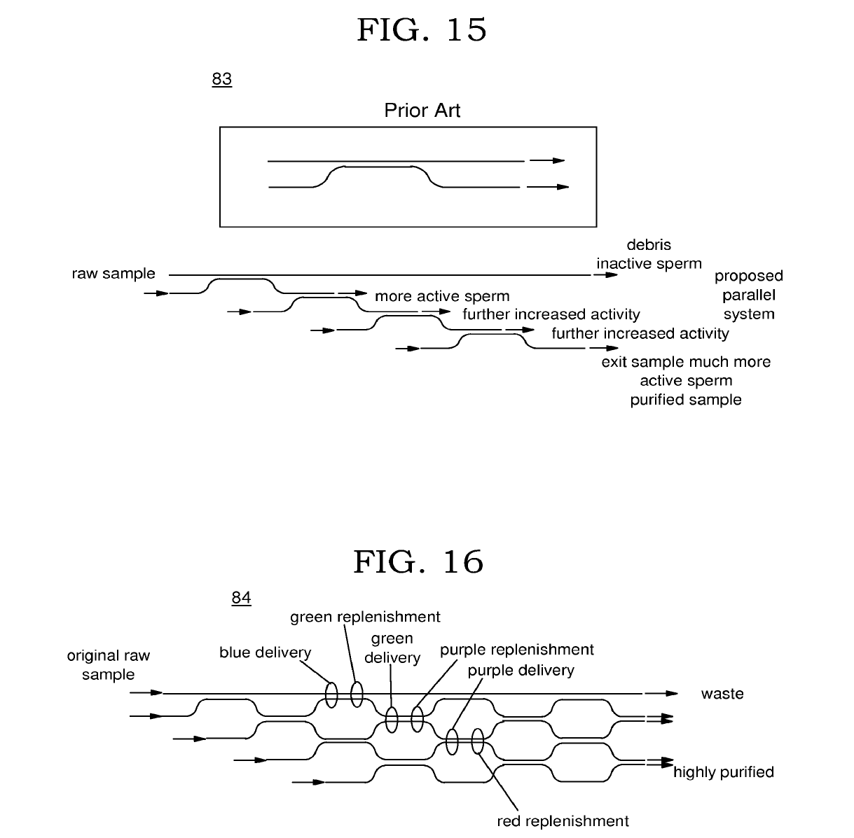

FIG. 15 shows the results of laminar flow in adjacent microfluidic channels.

FIG. 16 shows a network of laminar streams.

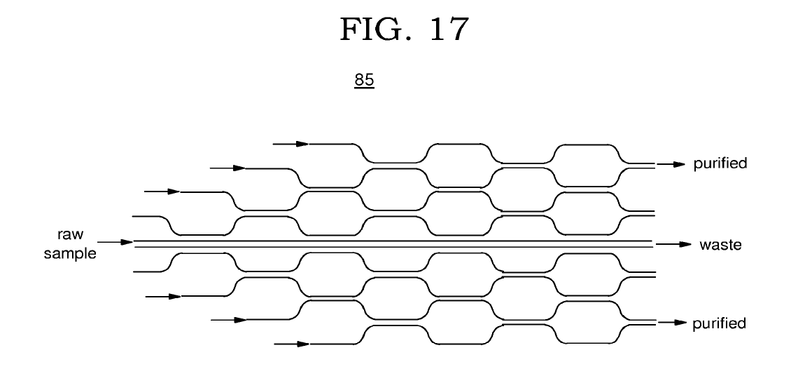

FIG. 17 shows a network wherein the network is repeated on both sides of a raw sample channel.

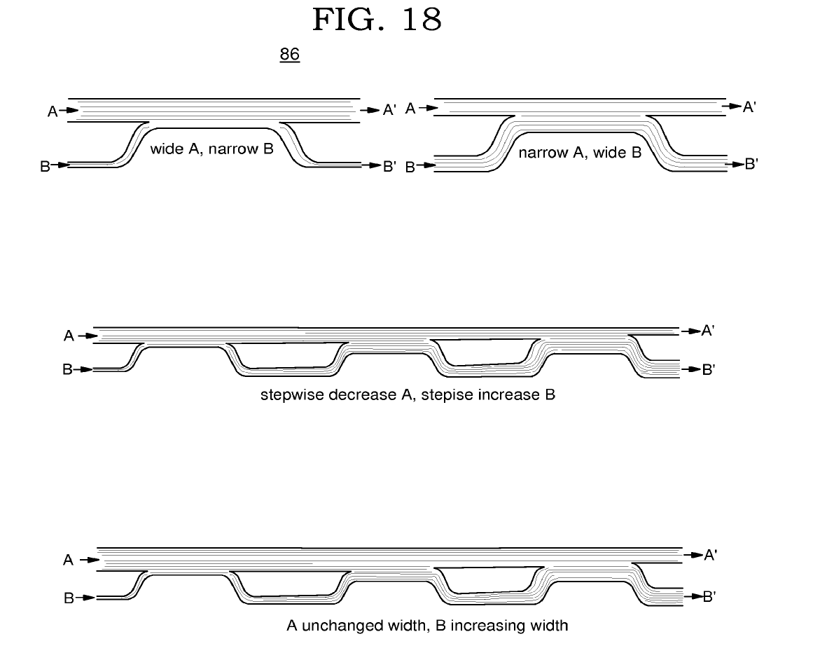

FIG. 18 shows a network with alternating the channel size or geometry.

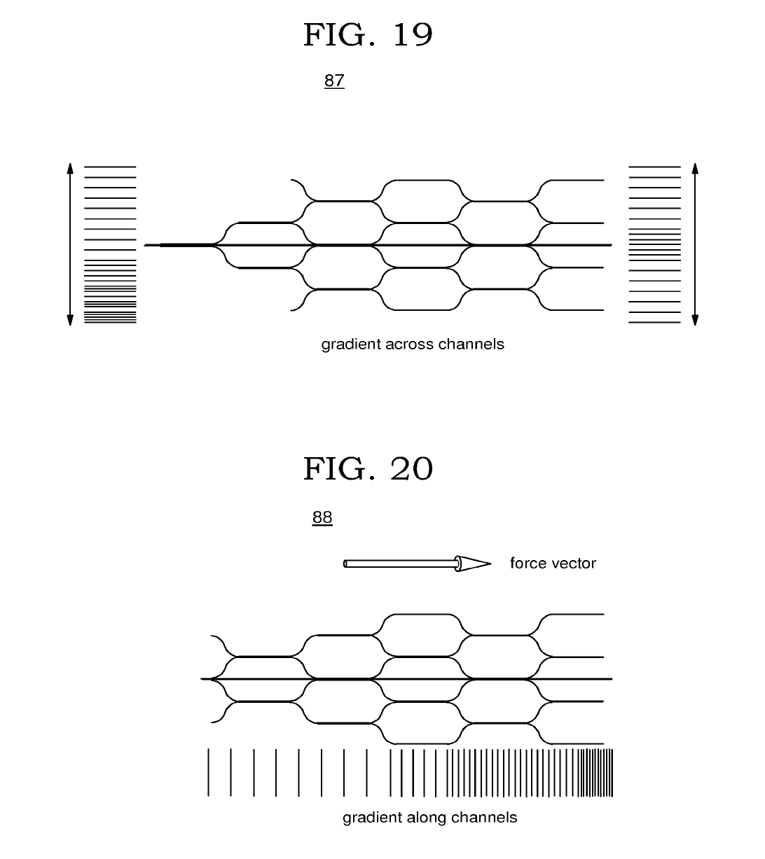

FIG. 19 shows a gradient across network channels.

FIG. 20 shows a gradient along network channels.

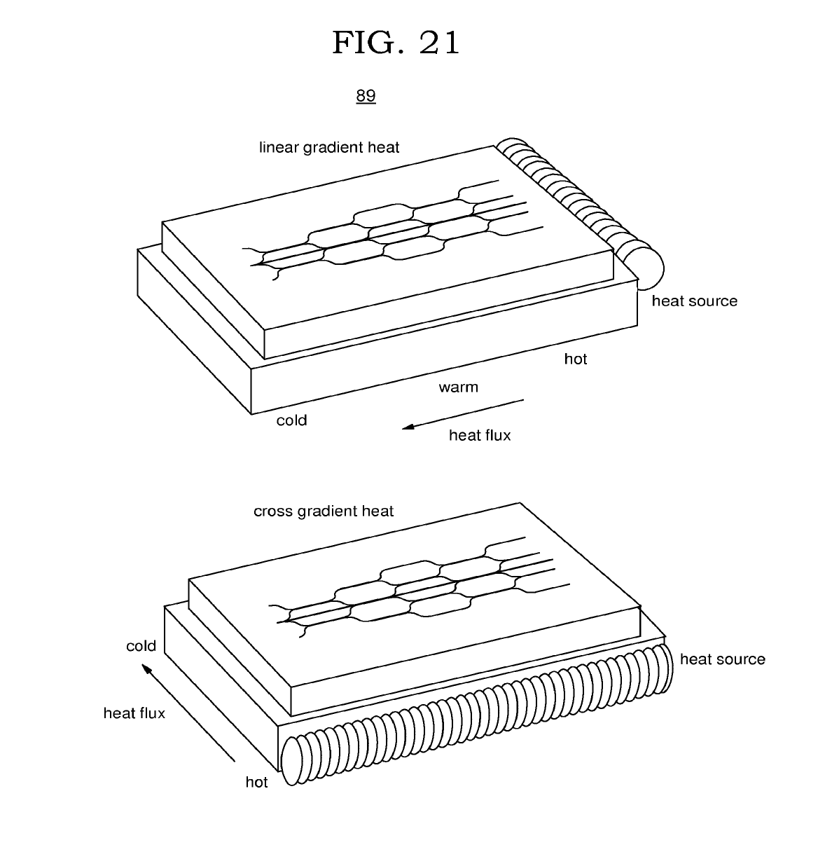

FIG. 21 shows a temperature gradient.

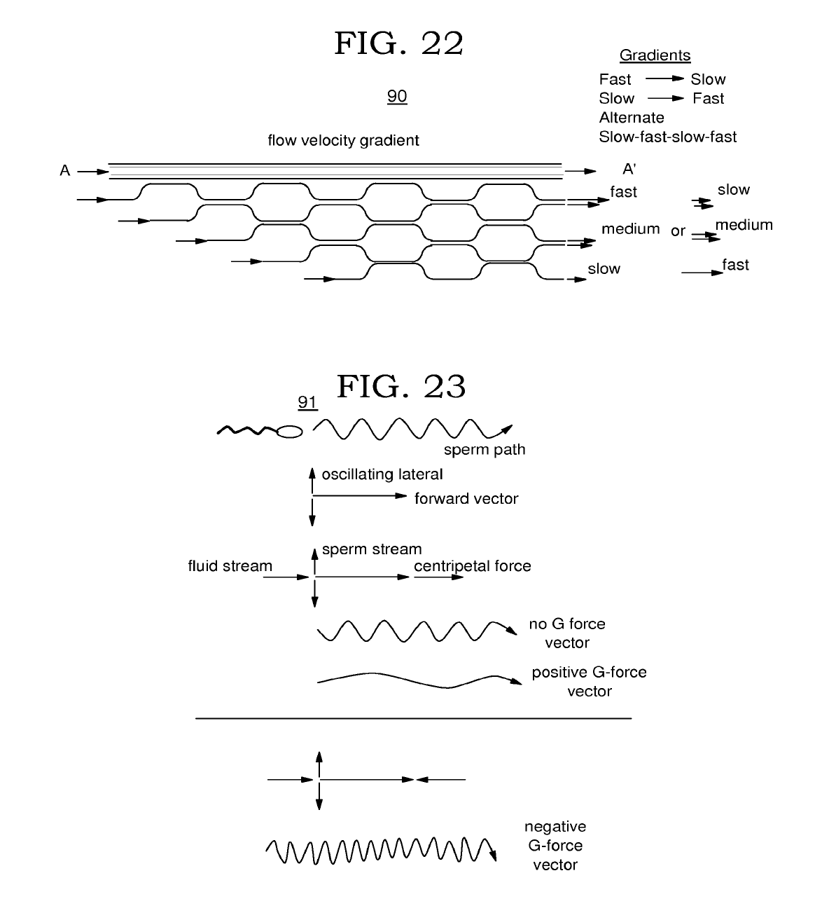

FIG. 22 shows a flow velocity gradient.

FIG. 23 shows the effect of centripetal force on sperm path.

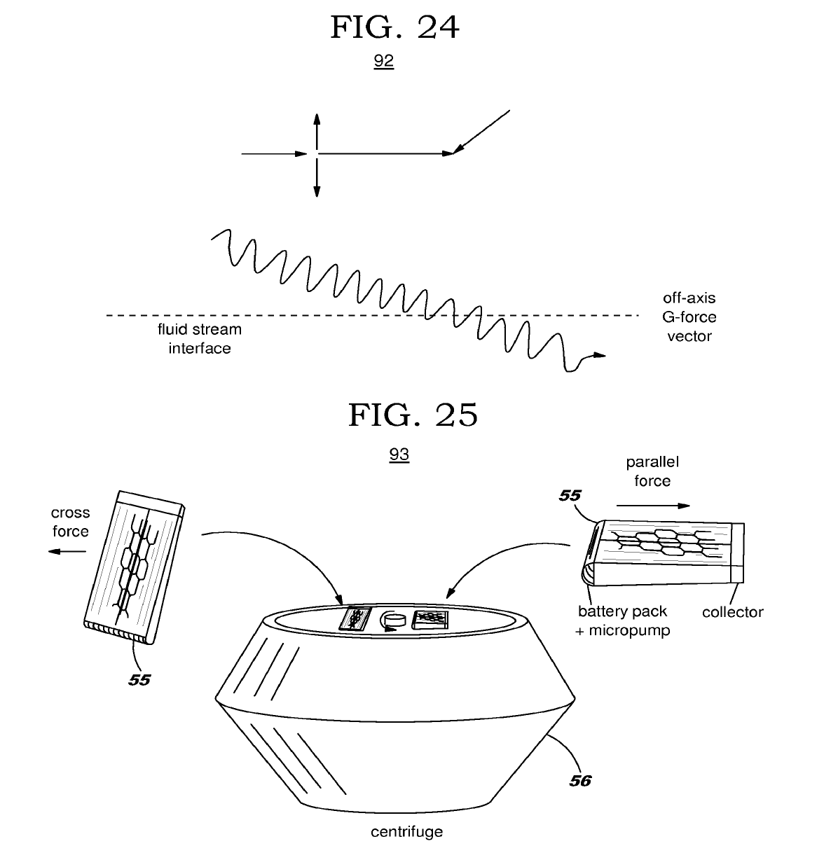

FIG. 24 shows the effect of centripetal force on sperm path.

FIG. 25 shows a method of centrifuging sperm in a microfluidic chip.

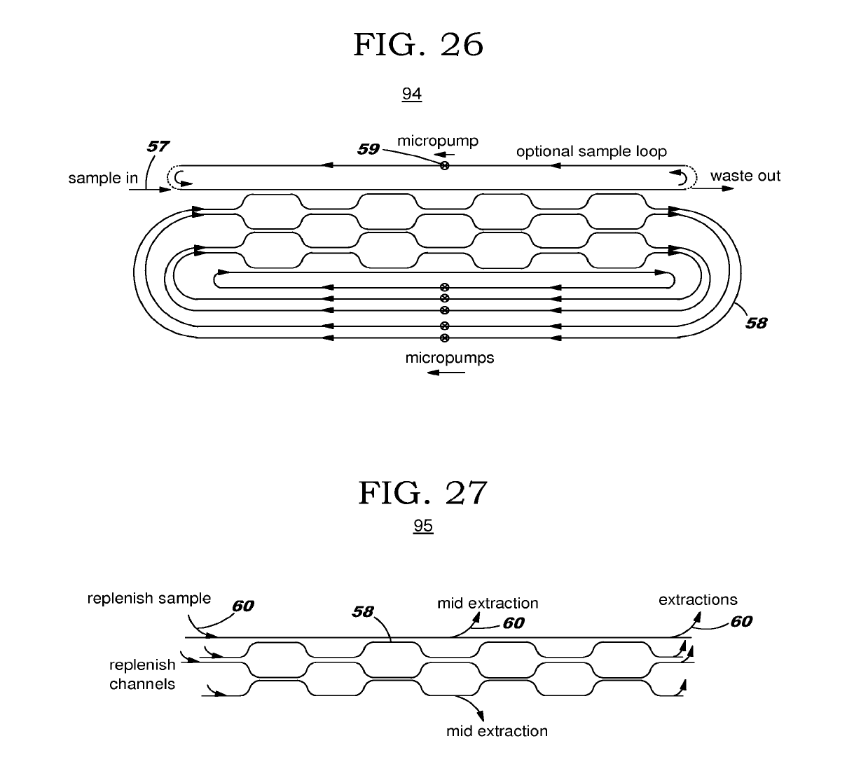

FIG. 26 shows a looped laminar flow channel system.

FIG. 27 shows a laminar flow channel system with side channels.

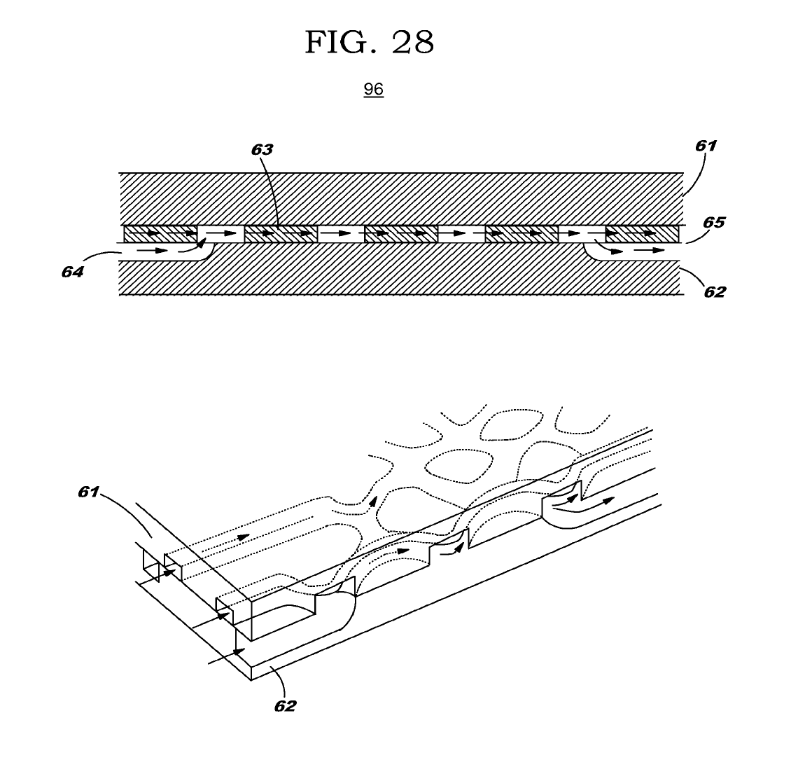

FIG. 28 shows two plates, each containing microchannels, fused together.

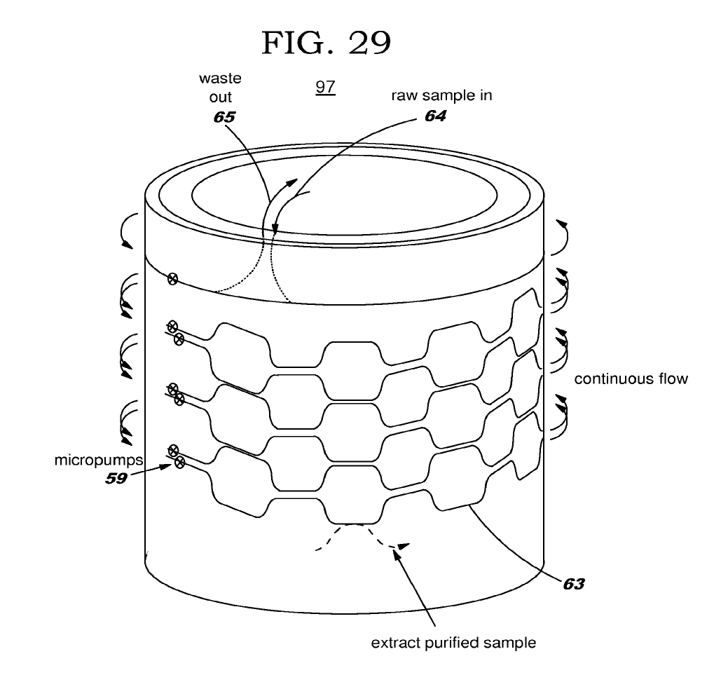

FIG. 29 shows a cylindrical network.

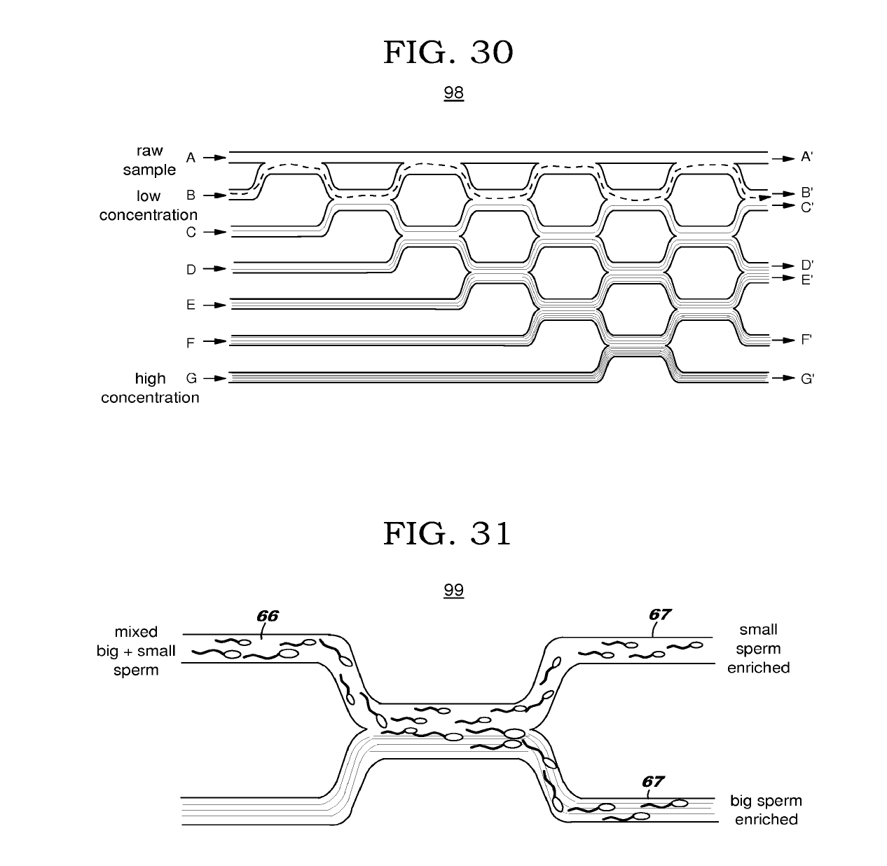

FIG. 30 shows a gradient laminar flow channel system.

FIG. 31 shows the effect of the system of FIG. 30 on sperm.

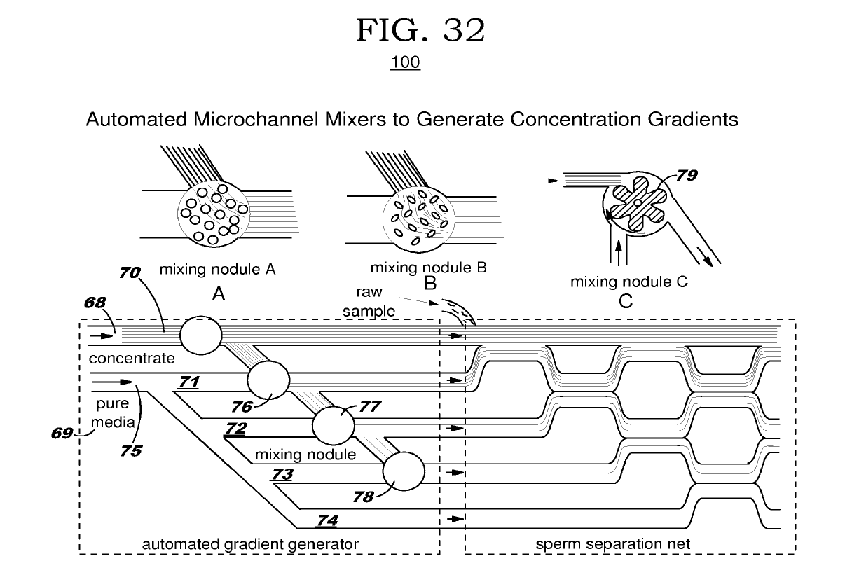

FIG. 32 shows the system of FIG. 30 with various mixing nodules.

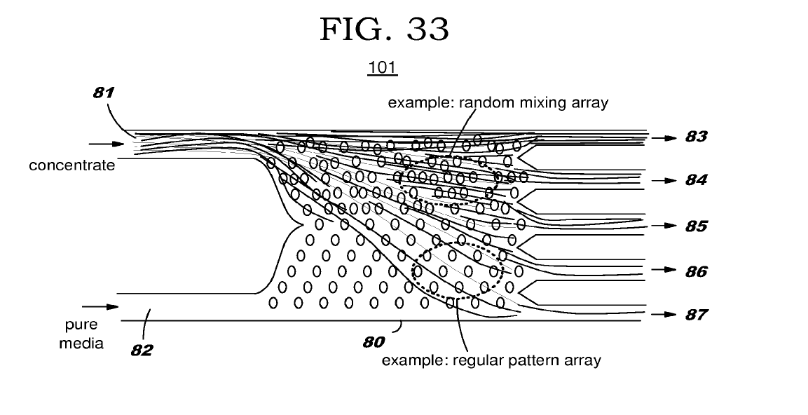

FIG. 33 shows an alternate single chamber mixing nodule.

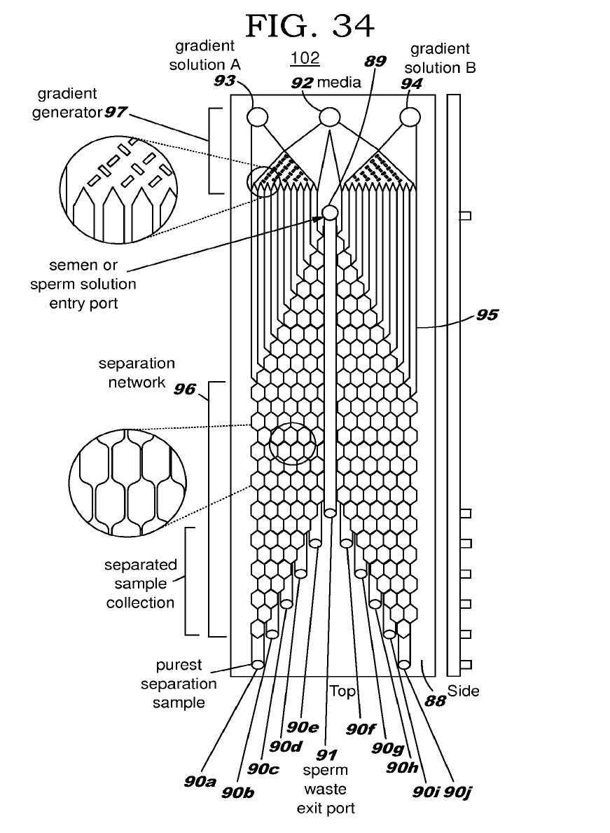

FIG. 34 shows a single microfluidic chip with all the basic components of a sperm separation system.

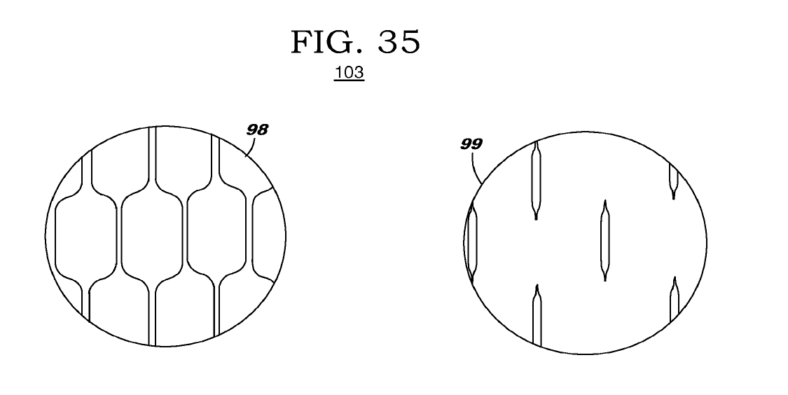

FIG. 35 shows alternate net configurations.

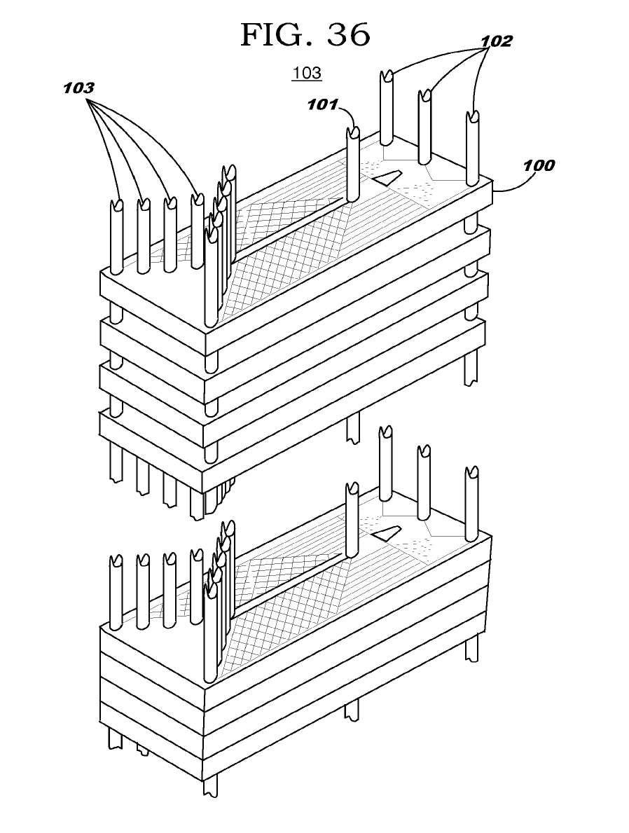

FIG. 36 shows multiple chips operating in parallel fashion.

FIG. 37 shows external forces which may be applied to a fractional distillation network.

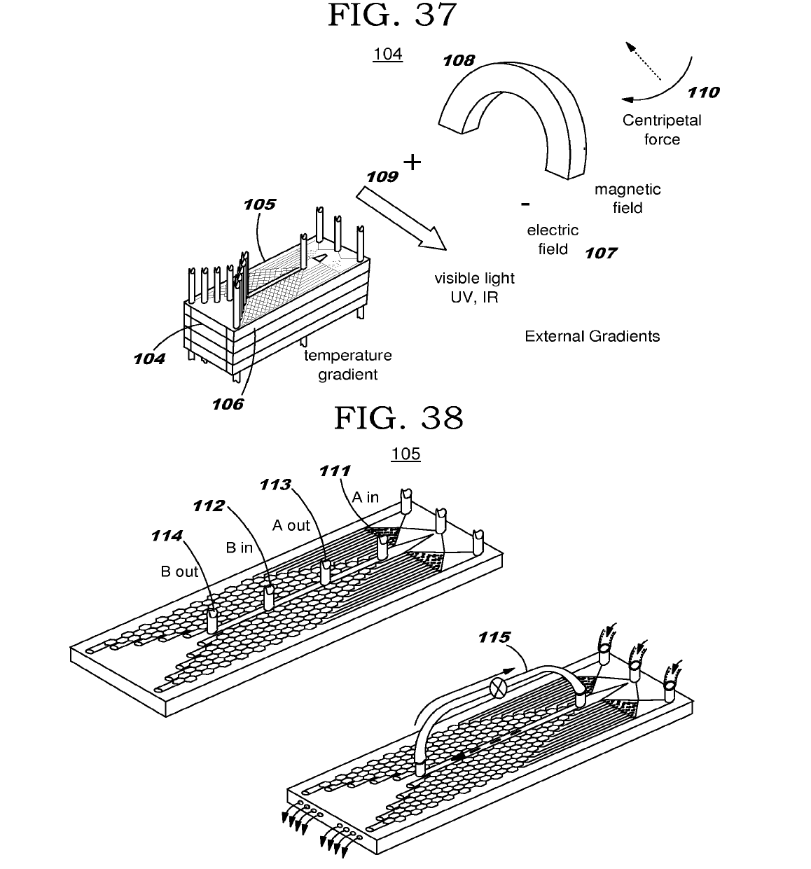

FIG. 38 shows variations in sperm separation chip design.

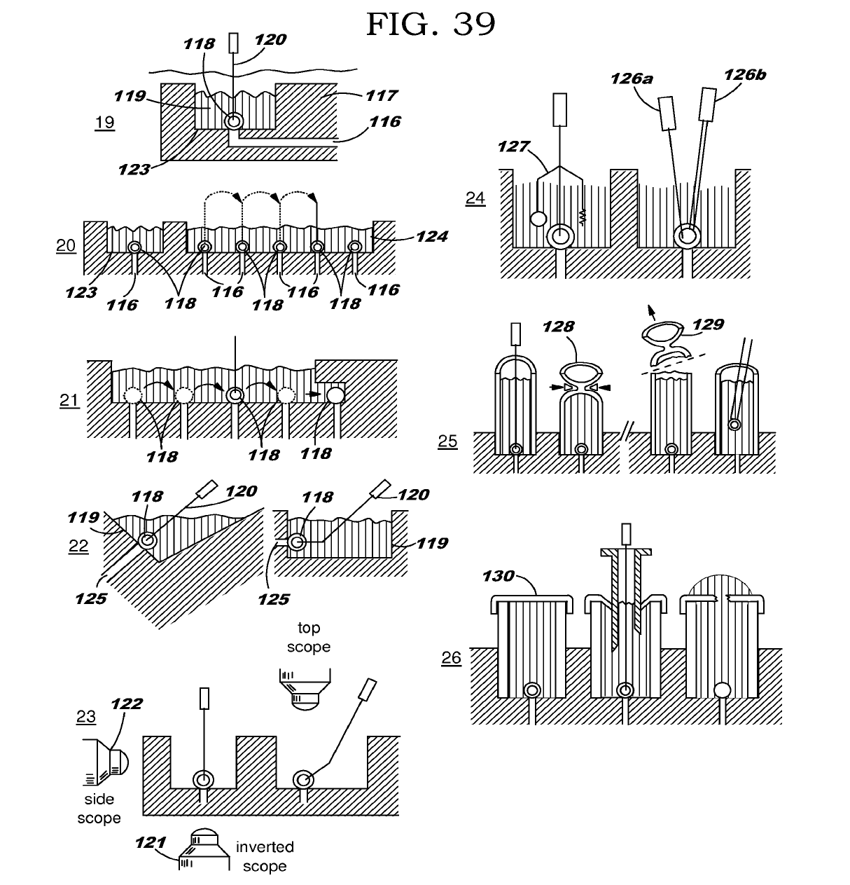

FIG. 39 shows a vertical micromanipulation system.

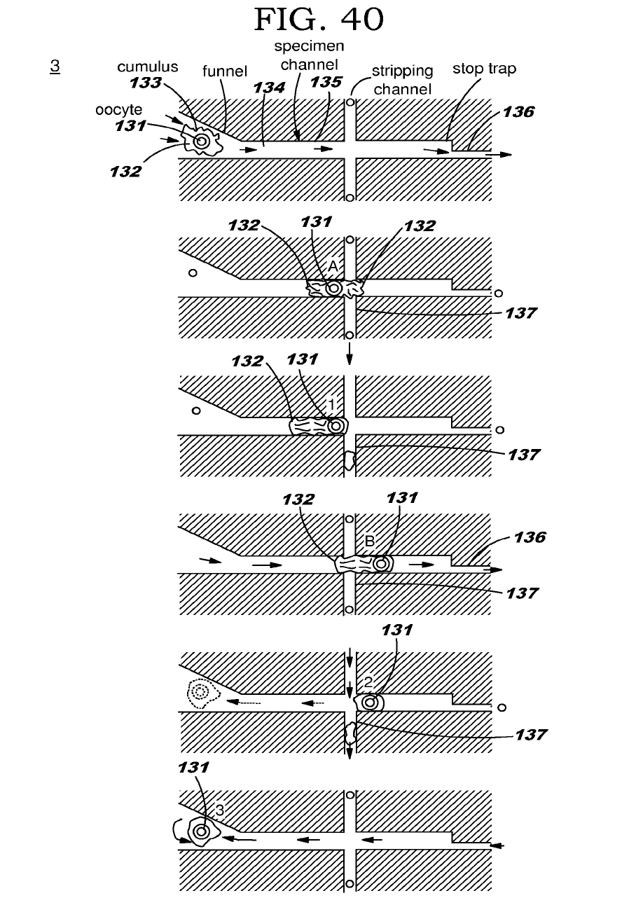

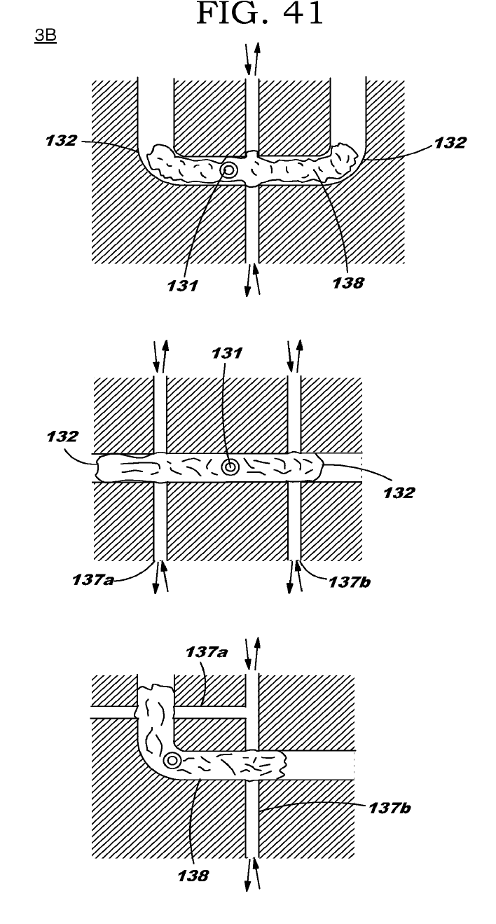

FIG. 40 shows a microfluidic oocyte stripping method.

FIG. 41 shows alternate microfluidic oocyte stripping configurations.

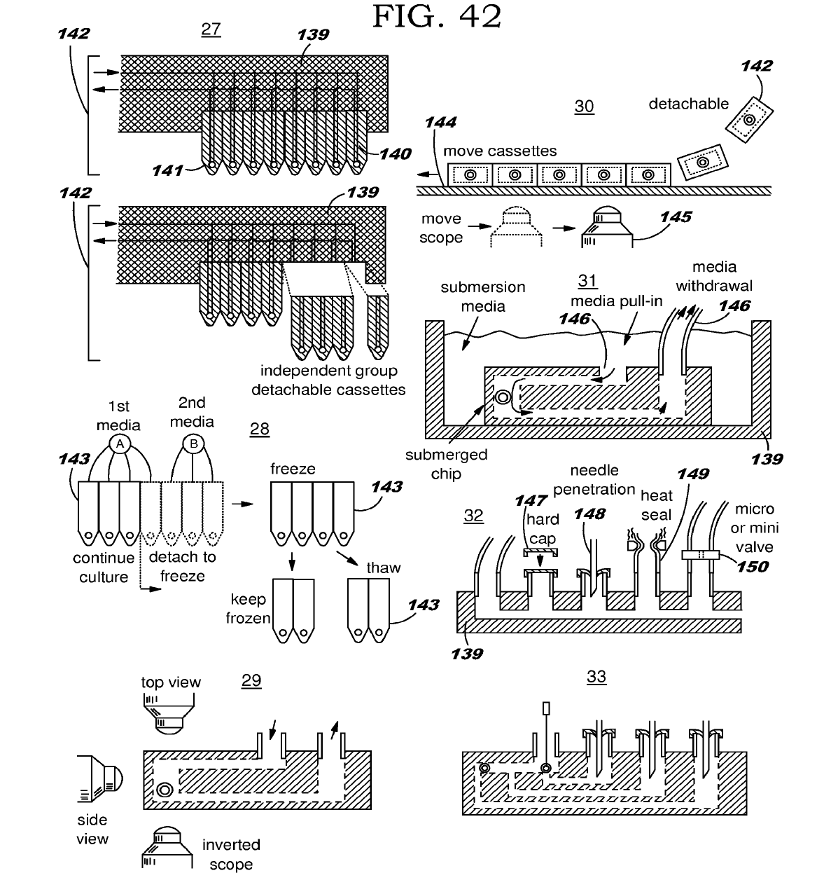

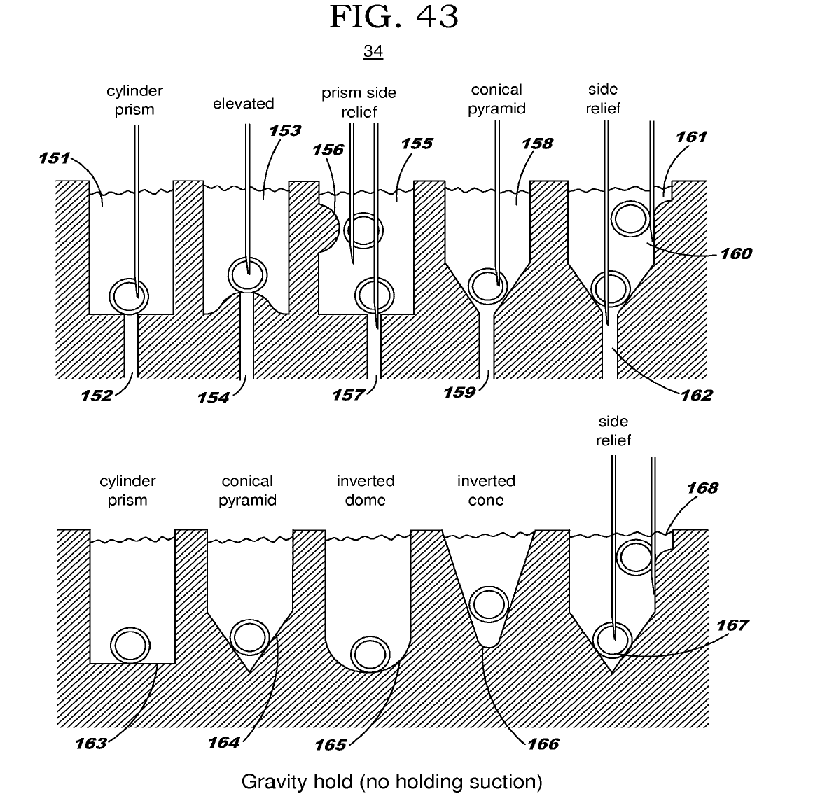

FIG. 42 shows a microfluidic cassette cell/tissue culture system.

FIG. 43 shows a variety of well shapes.

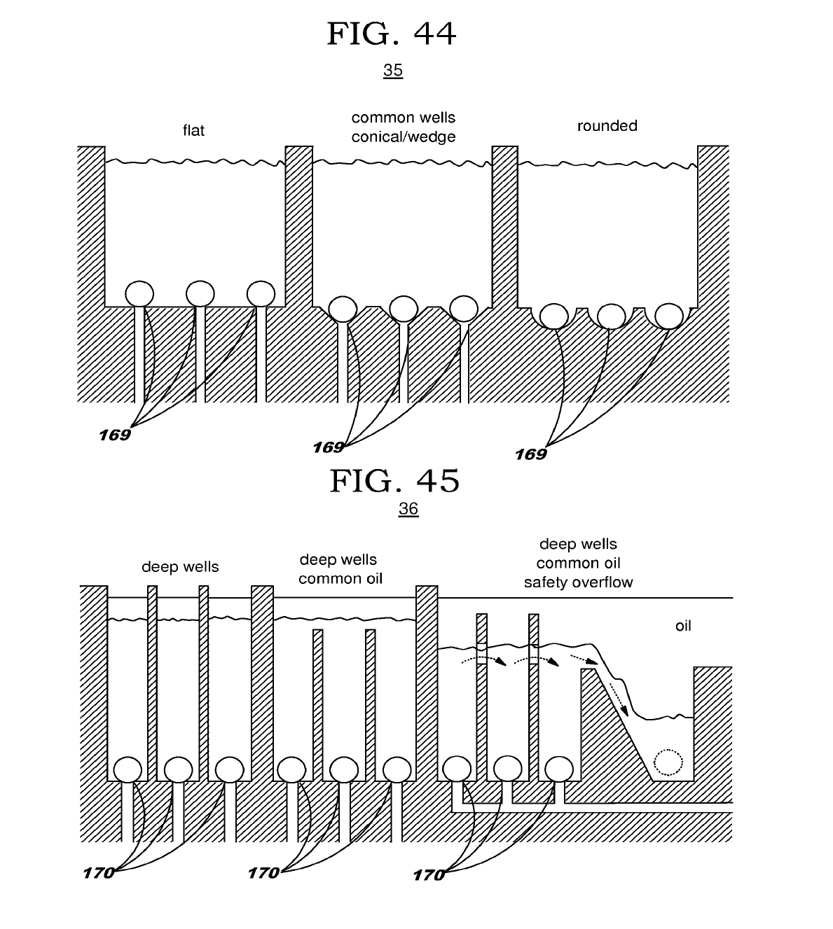

FIG. 44 shows common wells with a variety of well shapes.

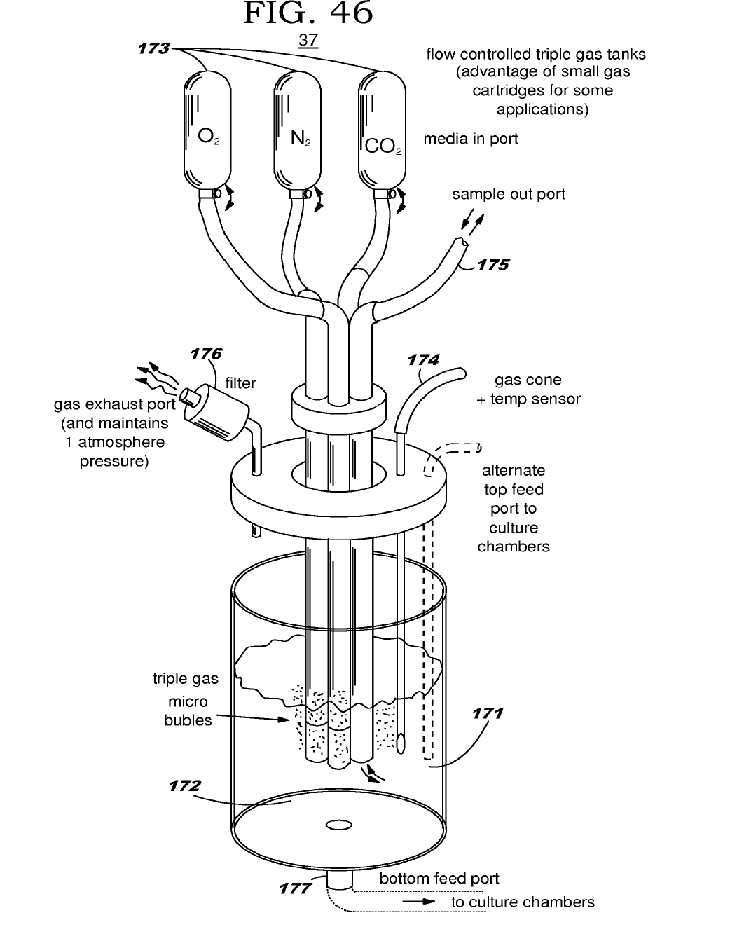

FIG. 45 shows a variety of deep wells.

FIG. 46 shows a system to supply culture media to the microfluidic system.

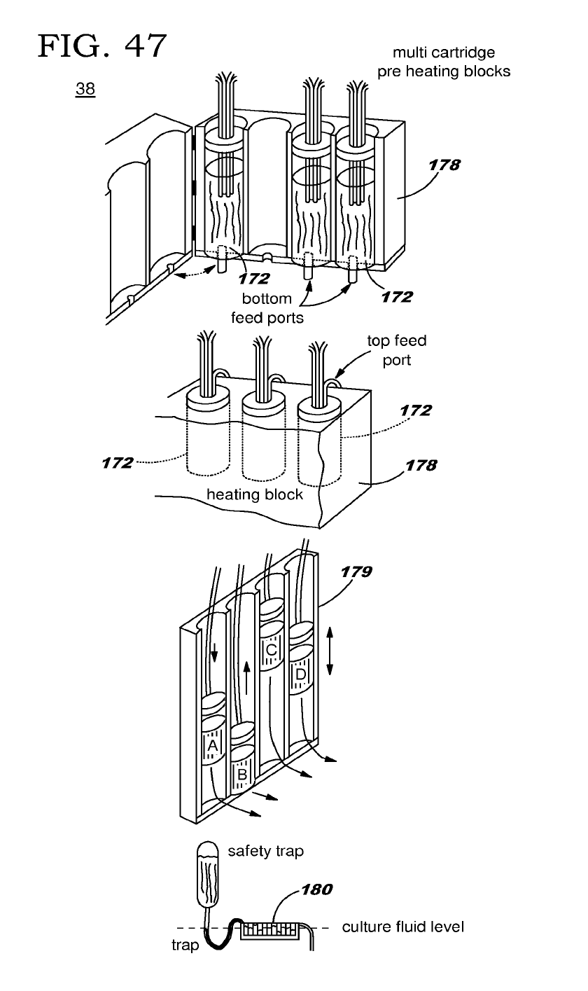

FIG. 47 shows preheating of media, adjustment to media flow and a safety trap for the system of FIG. 46.

FIG. 48 shows an alternative embodiment of a culture media supply system.

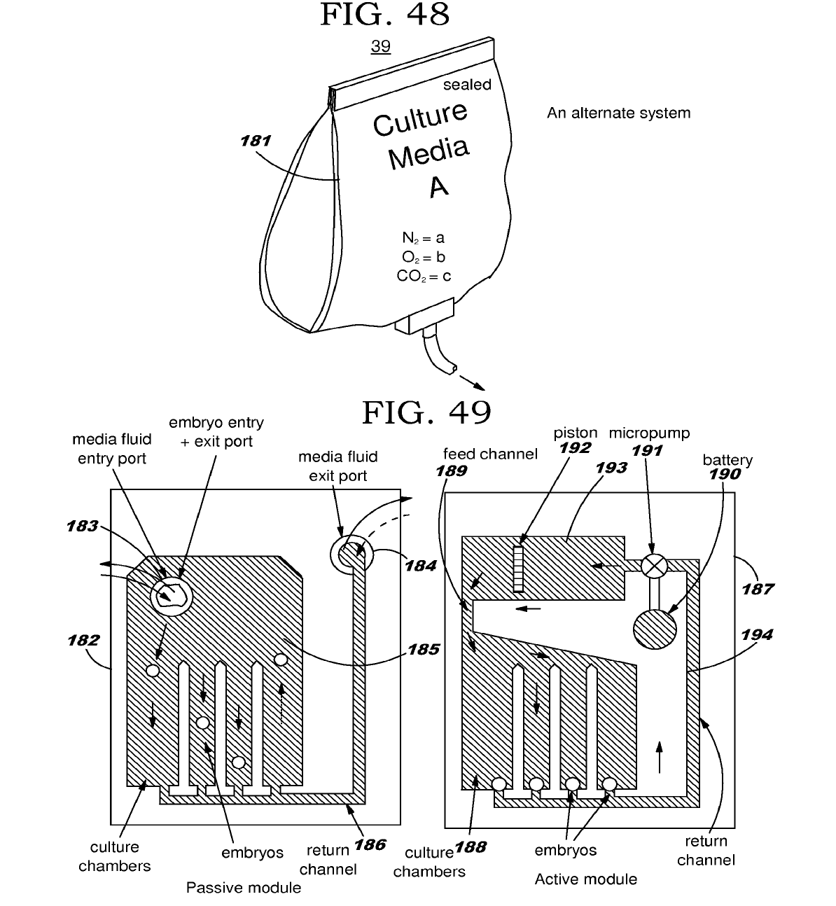

FIG. 49 shows a small microfluidic chip for embryo incubation.

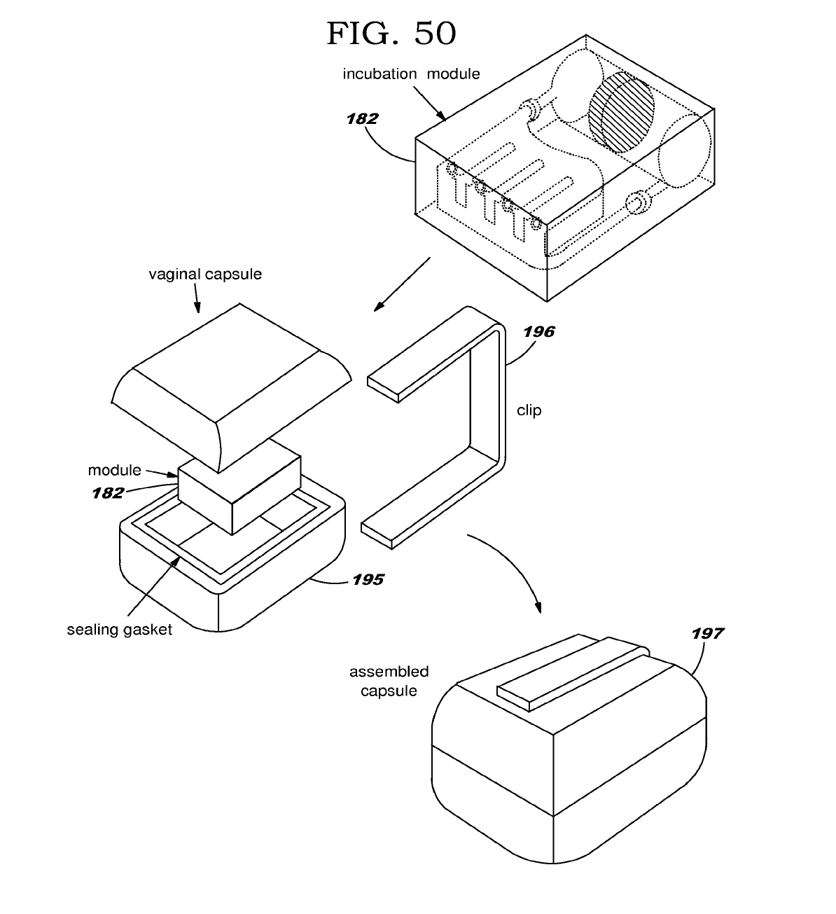

FIG. 50 shows an intra-vaginal embryo incubation module.

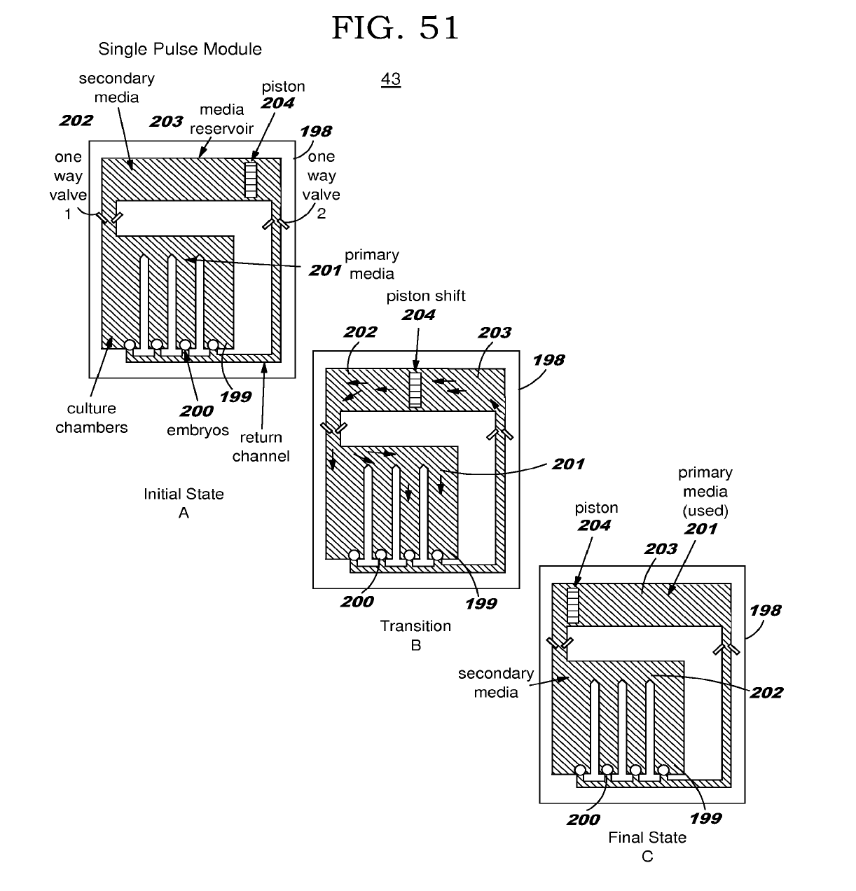

FIG. 51 shows a small microfluidic chip for embryo incubation containing two separate medias.

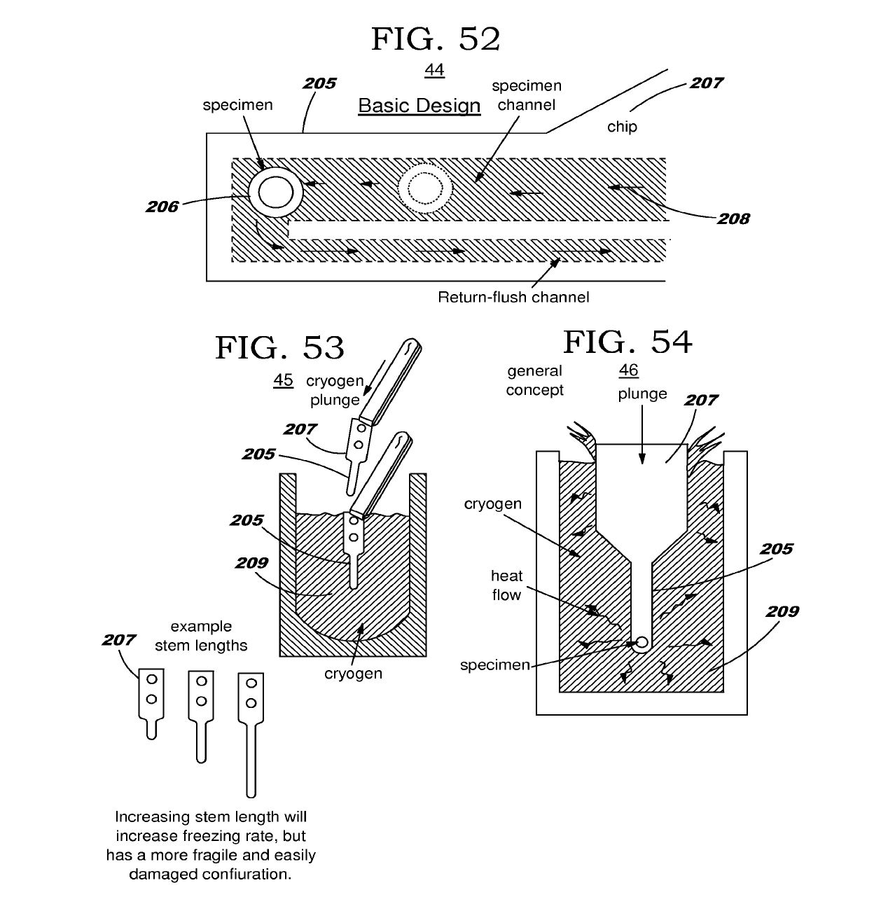

FIG. 52 shows a freezing stem of a microfluidic chip.

FIG. 53 shows a method for cryopreserving a specimen using the microfluidic chip of FIG. 52.

FIG. 54 shows a method for thawing a cryopreserved specimen using the microfluidic chip of FIG. 52.

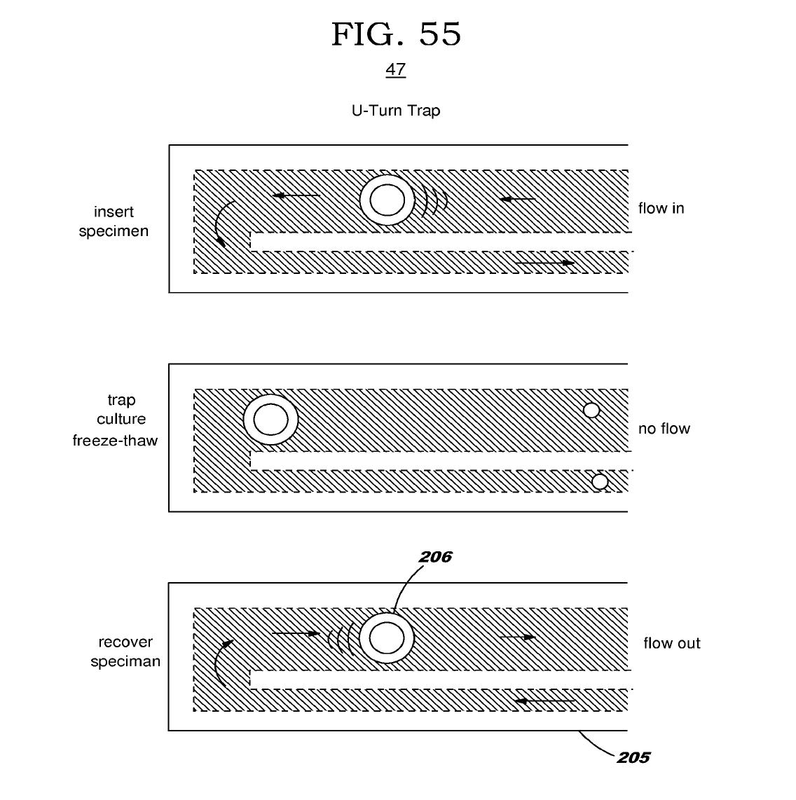

FIG. 55 shows retrieval of a specimen from the microfluidic chip of FIG. 52.

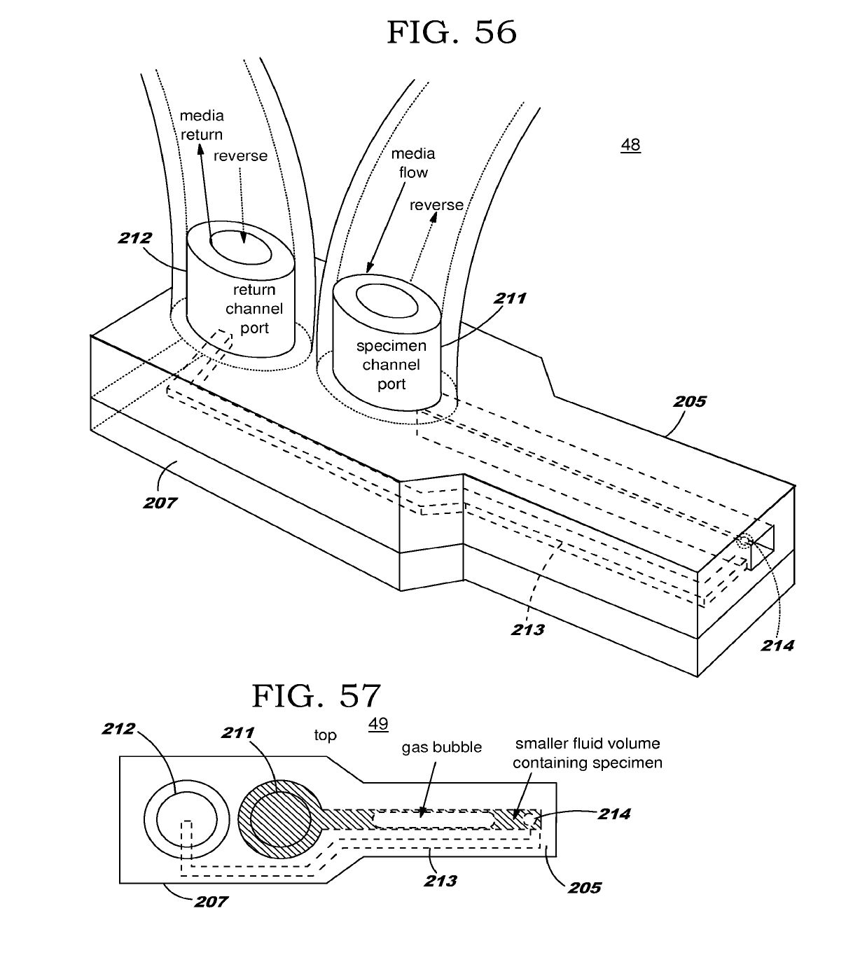

FIG. 56 is a perspective view of a microfluidic chip with a freezing stem.

FIG. 57 is a top view of the microfluidic chip shown in FIG. 56.

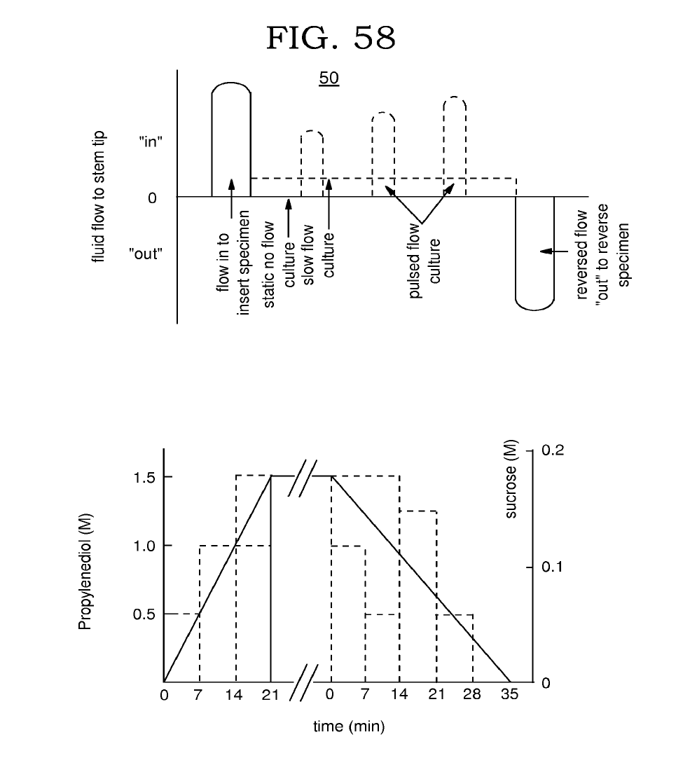

FIG. 58 describes application of cryoprotective solution concentration prior to cryopreservation.

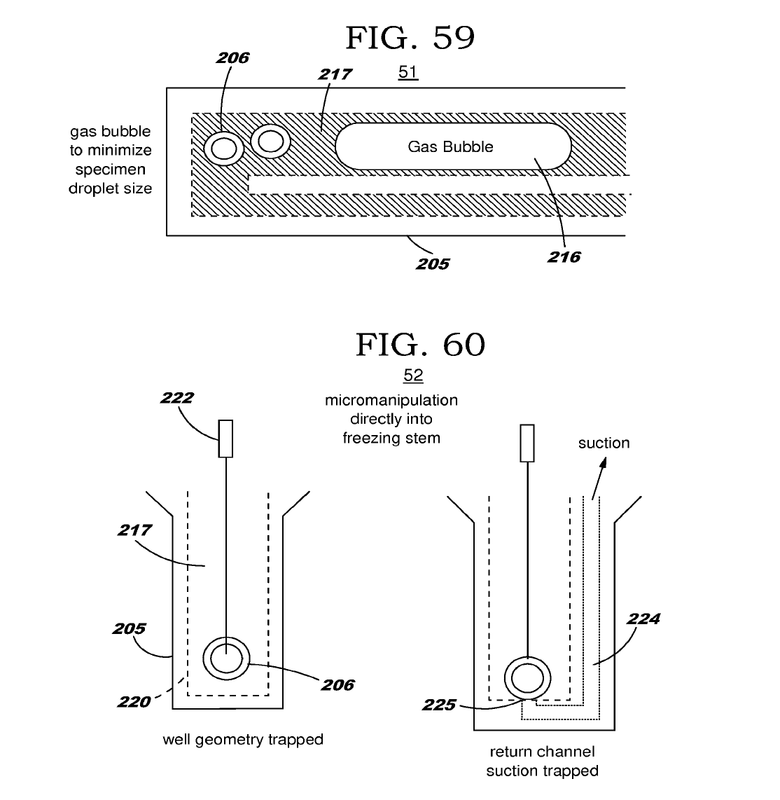

FIG. 59 shows introduction of a gas bubble to the specimen channel.

FIG. 60 shows micromanipulation of a specimen in a freezing stem.

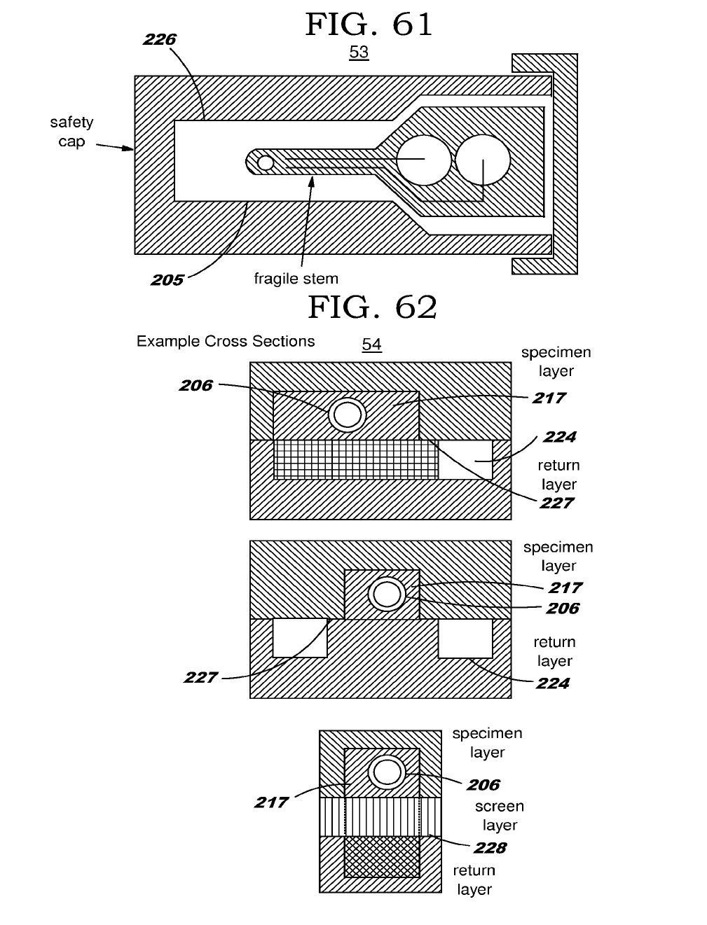

FIG. 61 shows a cap for a freezing stem.

FIG. 62 shows sample cross-sections of a freezing stem.

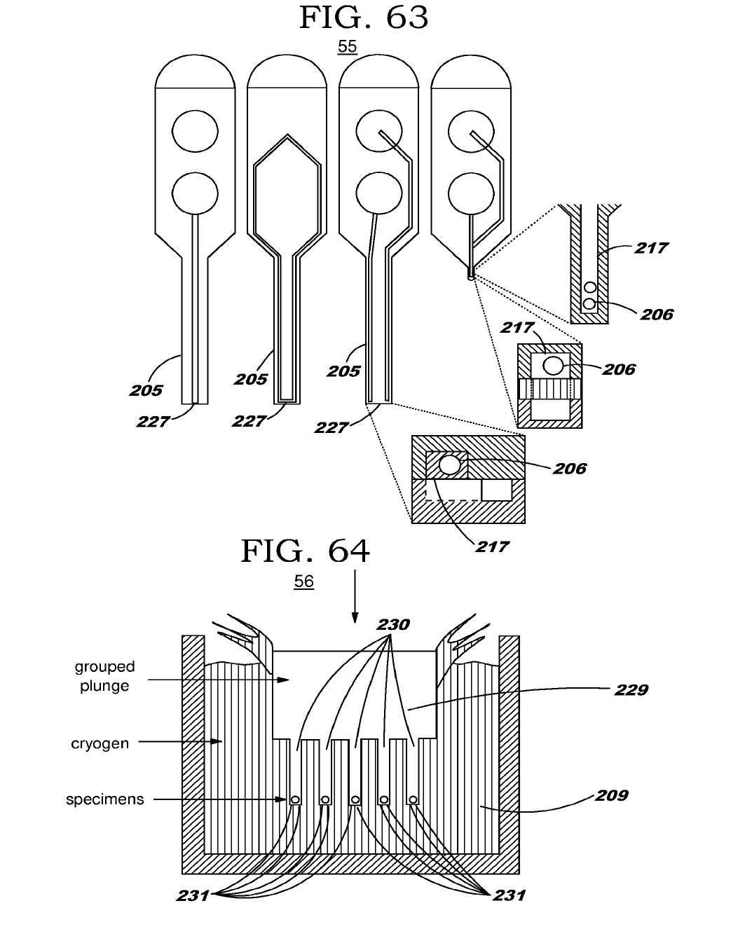

FIG. 63 shows freezing stems with single and double return channel designs, and long and short stem lengths.

FIG. 64 shows a microfluidic chip with multiple freezing stems.

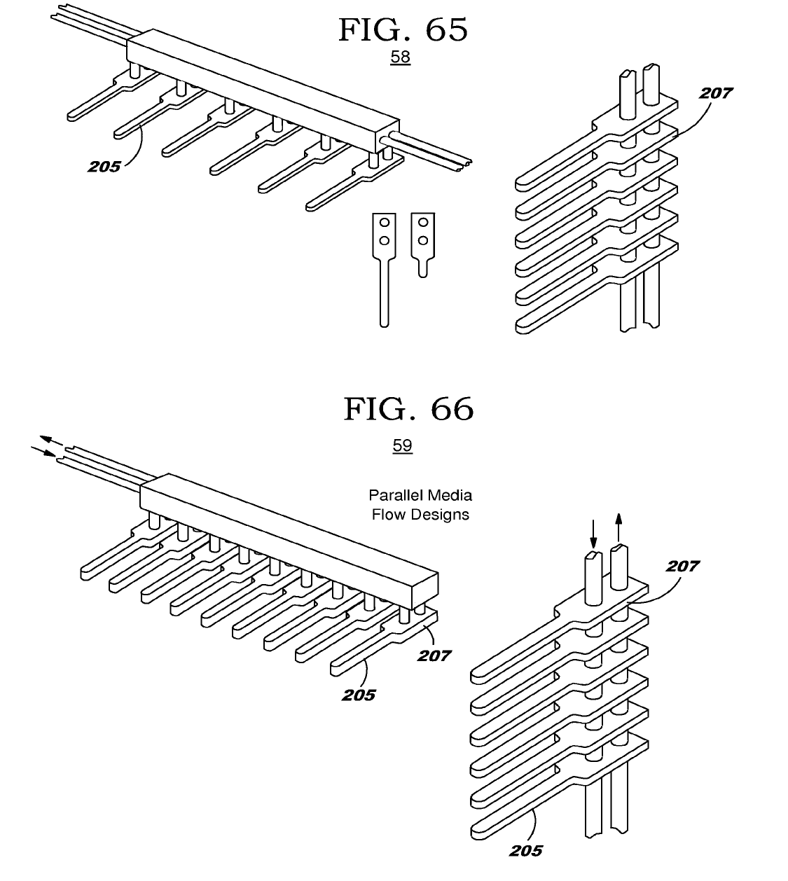

FIGS. 65 and 66 show rows and stacks of combined incubation and freezing stem units.

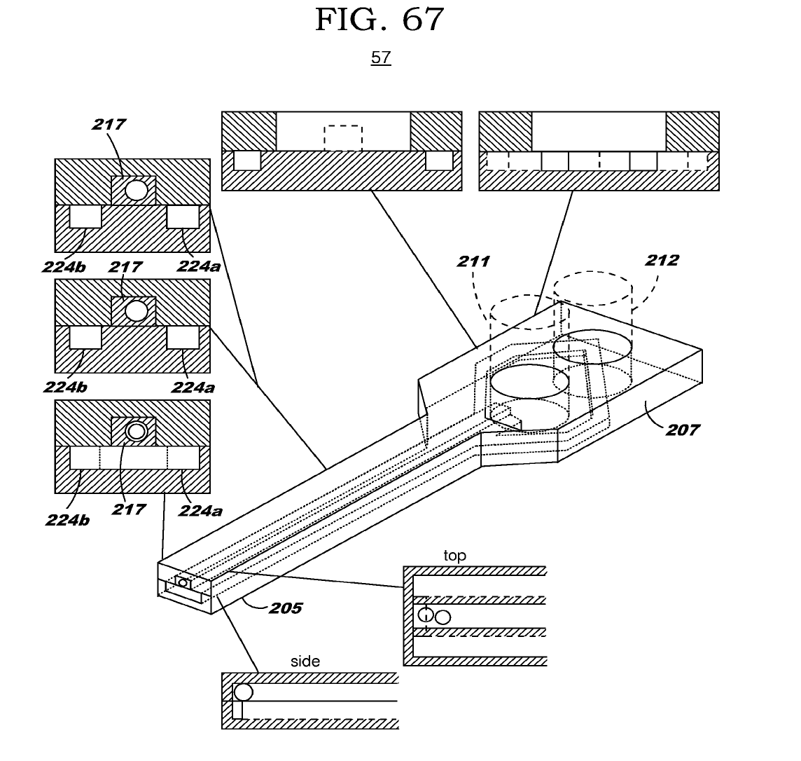

FIG. 67 shows an alternate embodiment of a freezing cassette.

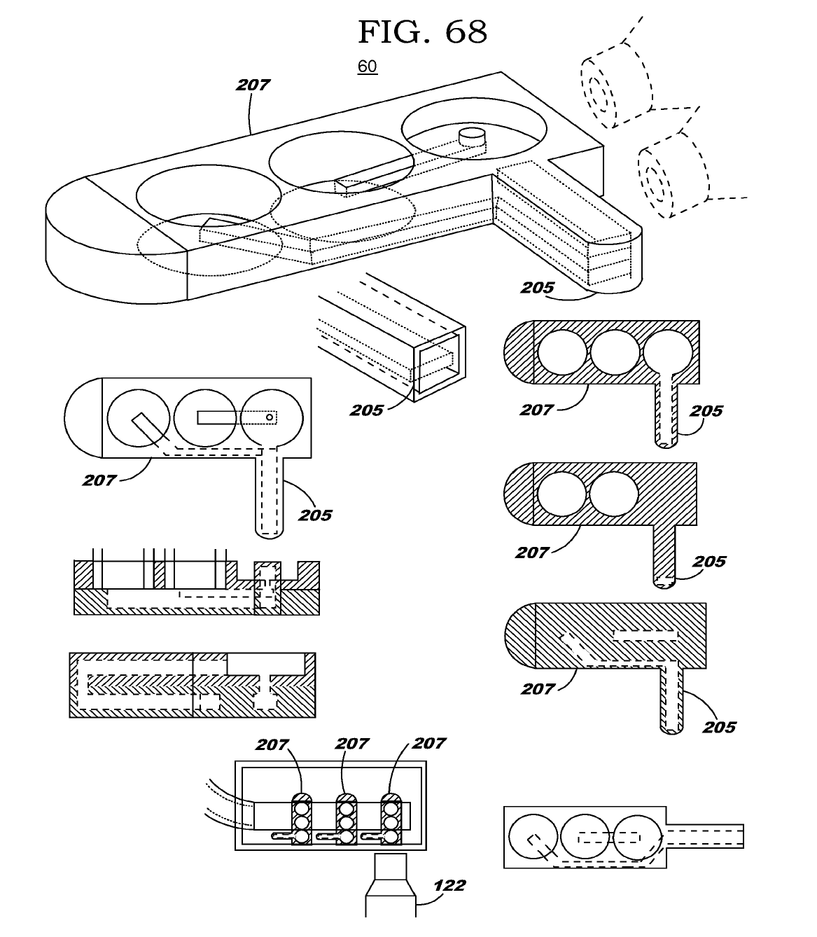

FIG. 68 shows a variation of the combined vertical micromanipulation, embryo incubation, cryopreservation microfluidic chip.

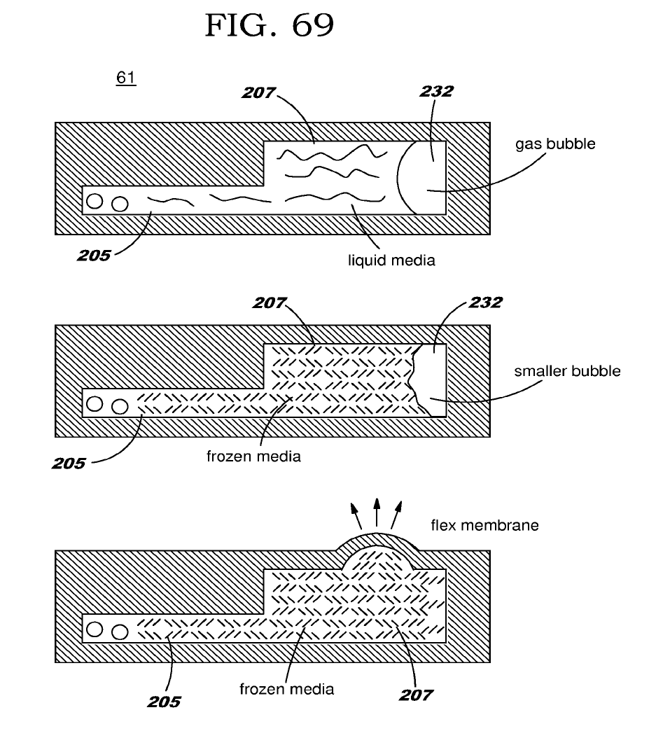

FIG. 69 shows an inert gas bubble in a cryopreservation microfluidic chip.

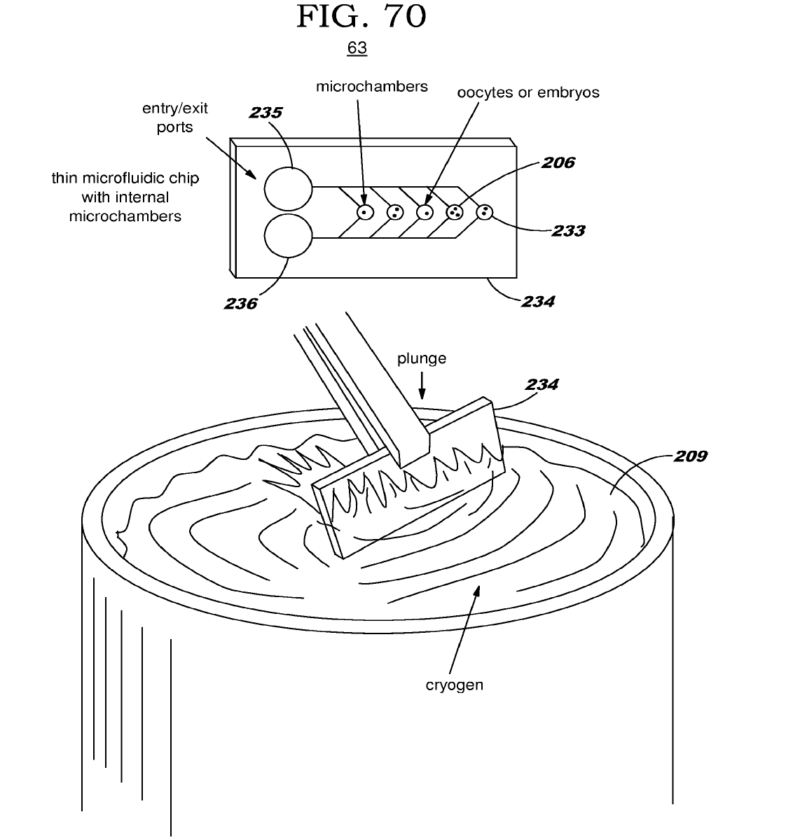

FIG. 70 shows an alternate embodiment of the freezing system.

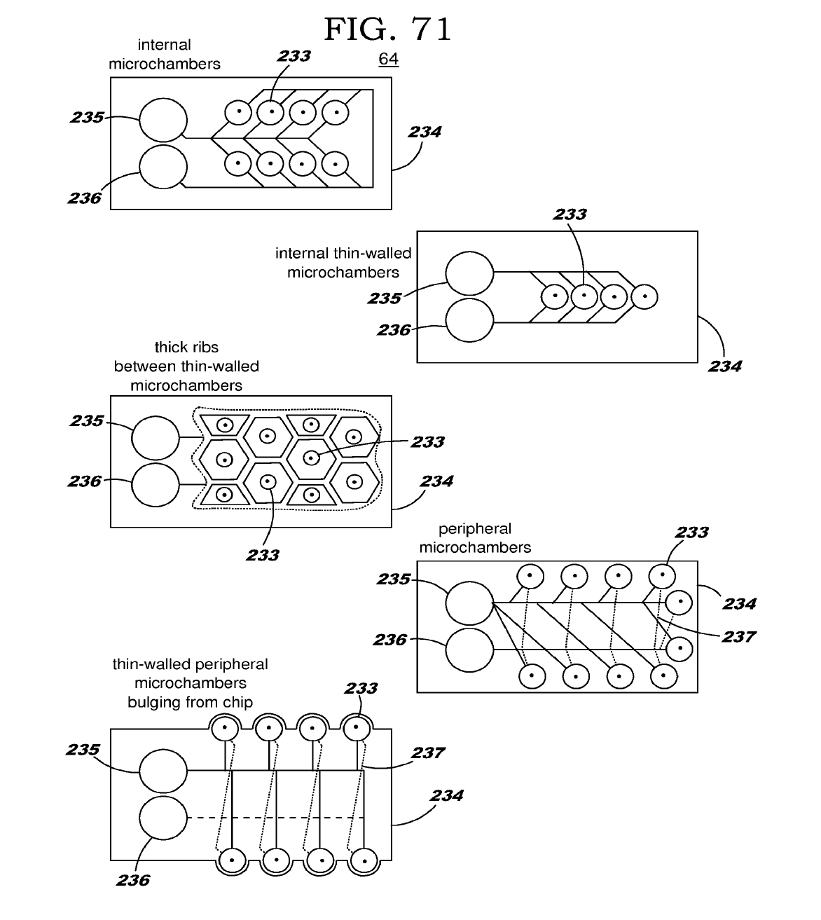

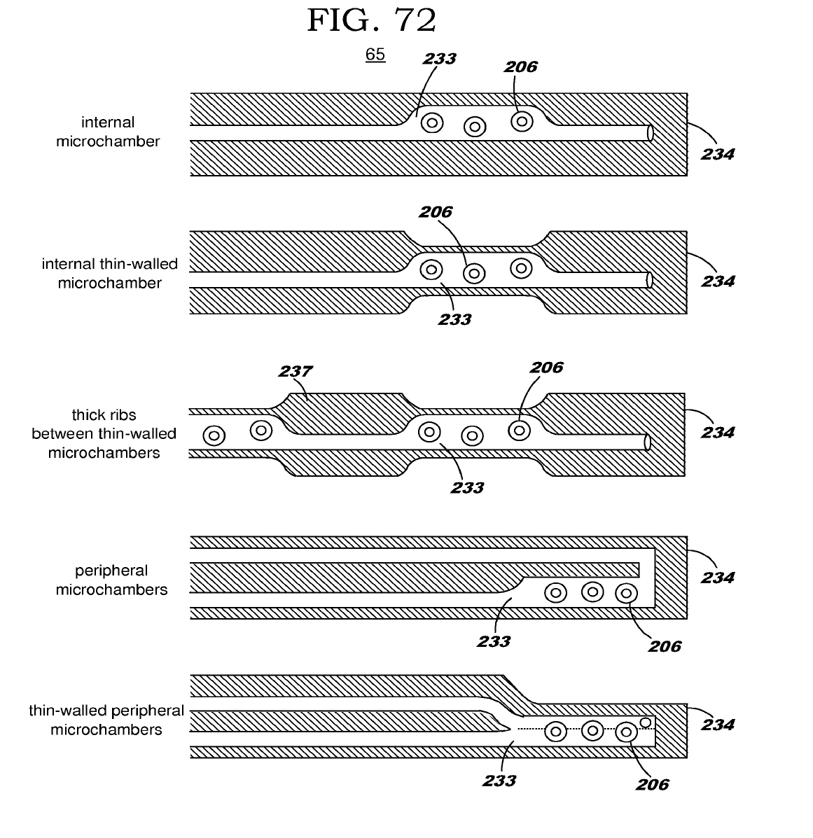

FIGS. 71 and 72 show multiple variations of the microfluidic chip of FIG. 70.

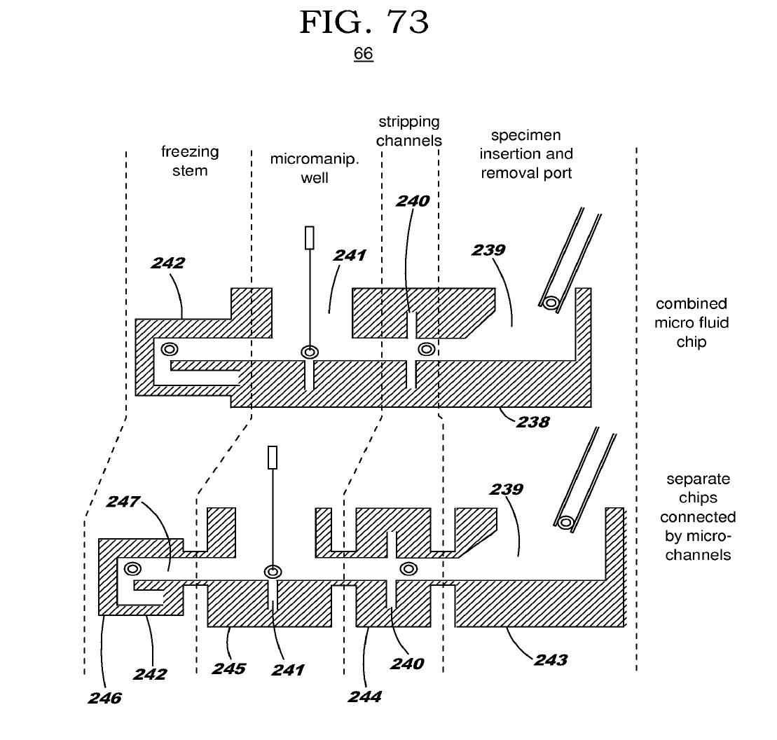

FIG. 73 shows a combined microfluid chip.

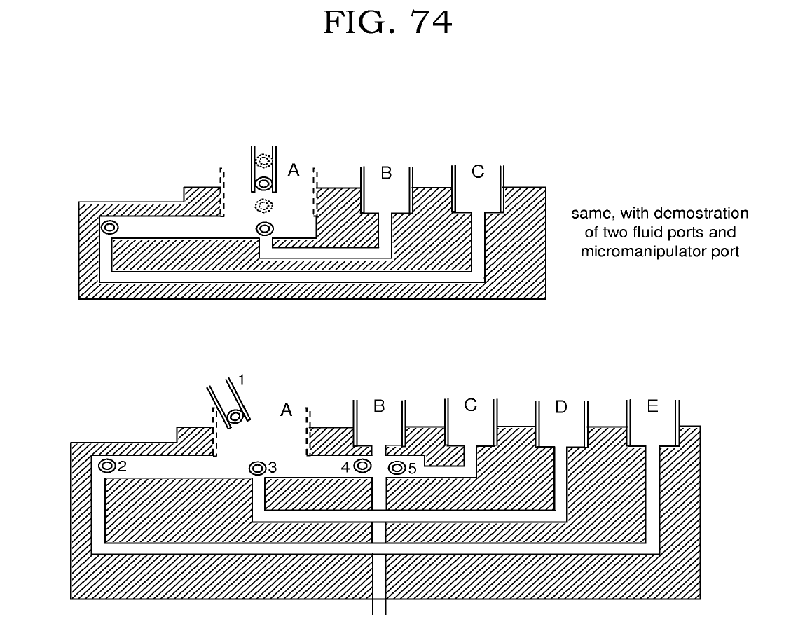

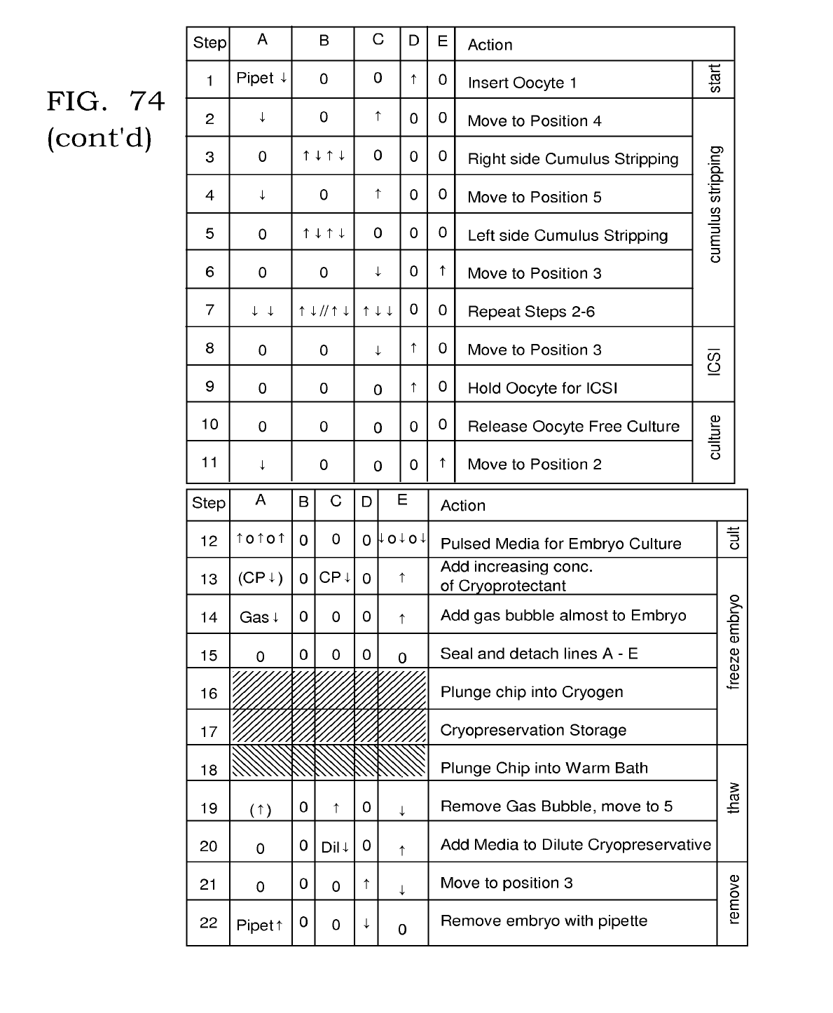

FIG. 74 shows many ways a single microfluid chip can be used.

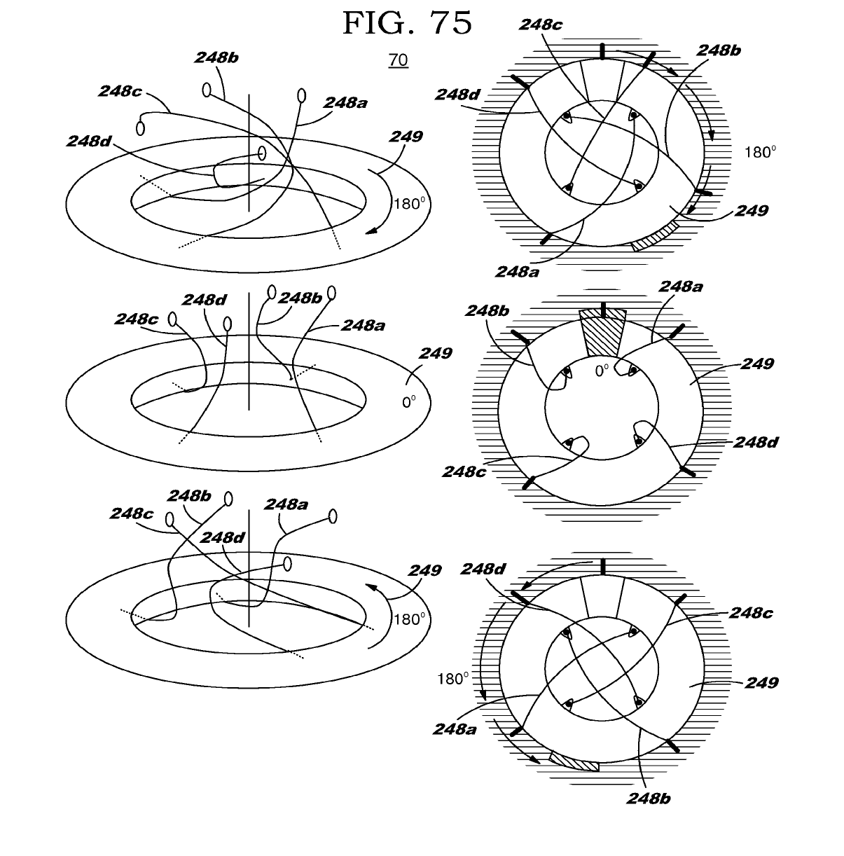

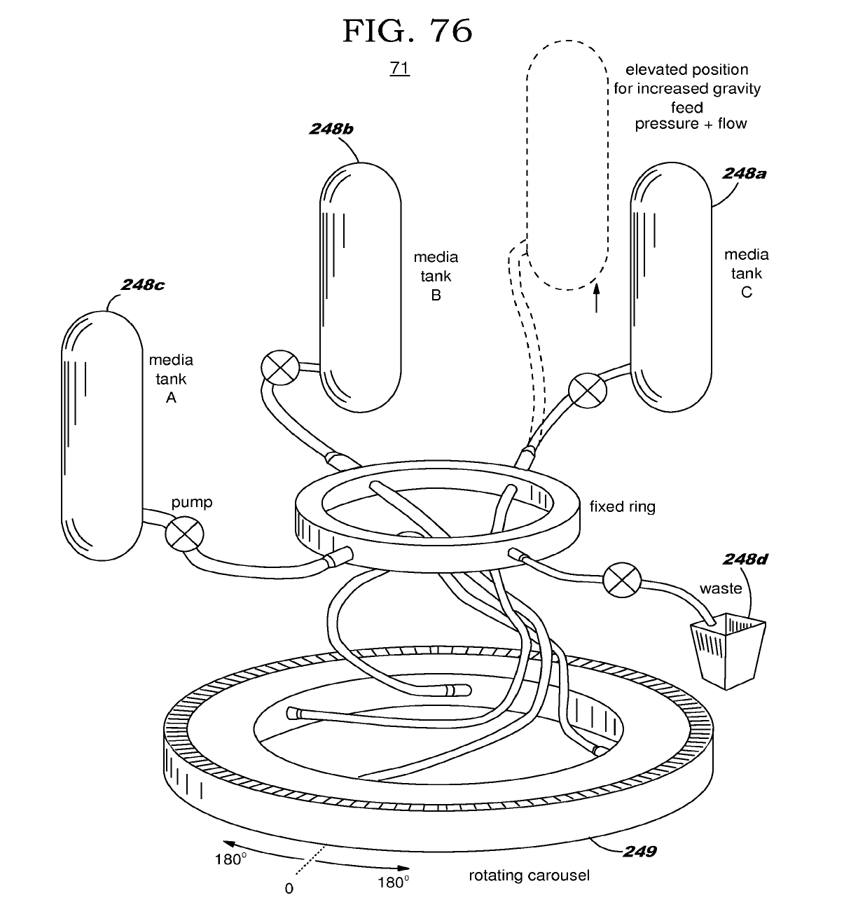

FIGS. 75 and 76 illustrate a simple media feed system.

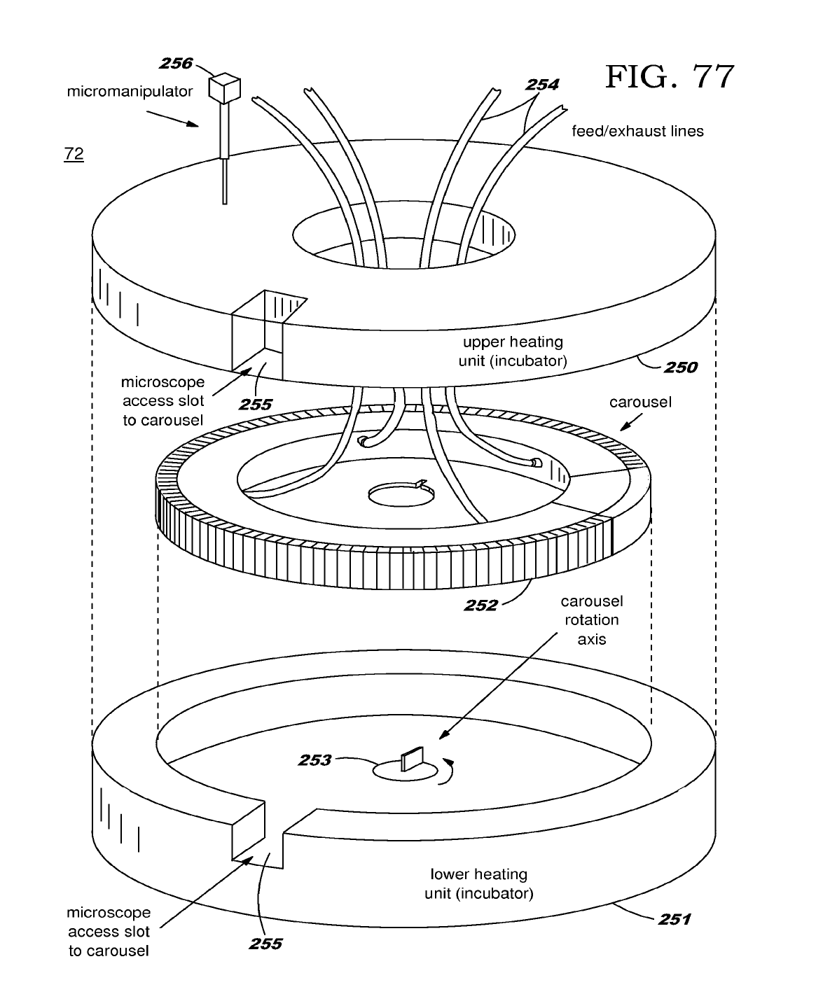

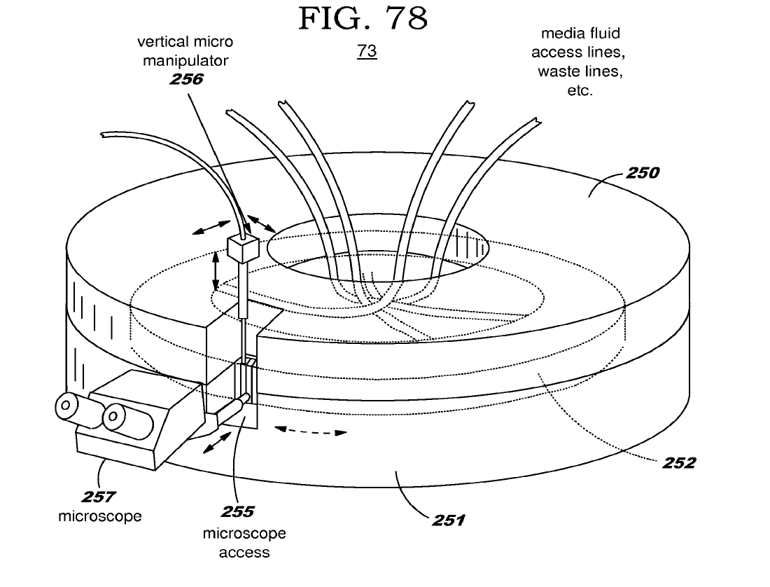

FIG. 77 is an exploded view of a robotic microfluidic incubator system.

FIG. 78 is a perspective view of the robotic microfluidic incubator system of FIG. 77.

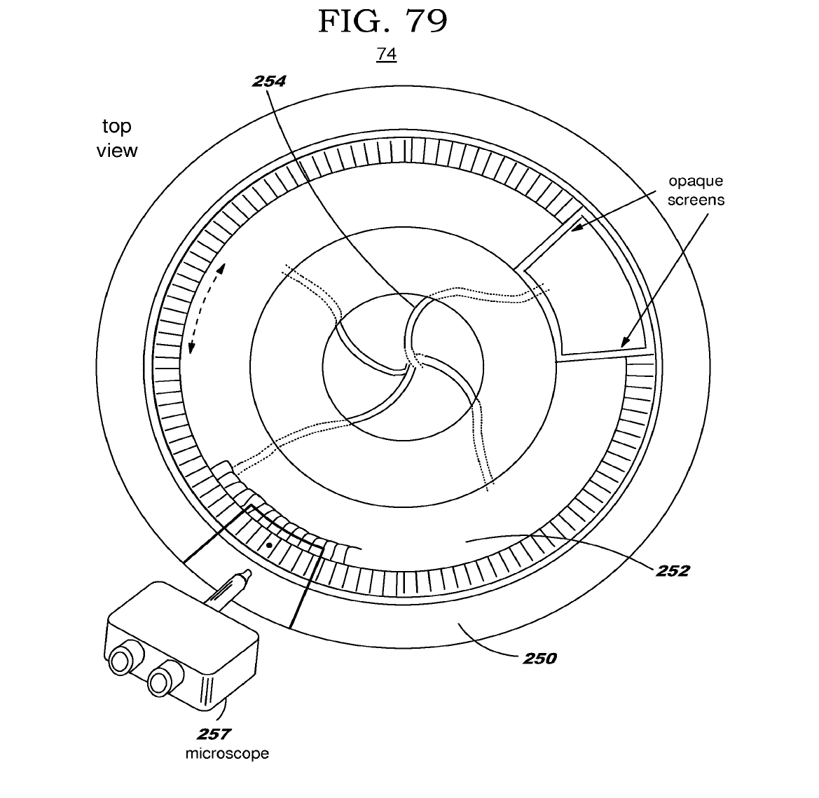

FIG. 79 is a top plan view of the robotic microfluidic incubator system of FIG. 77.

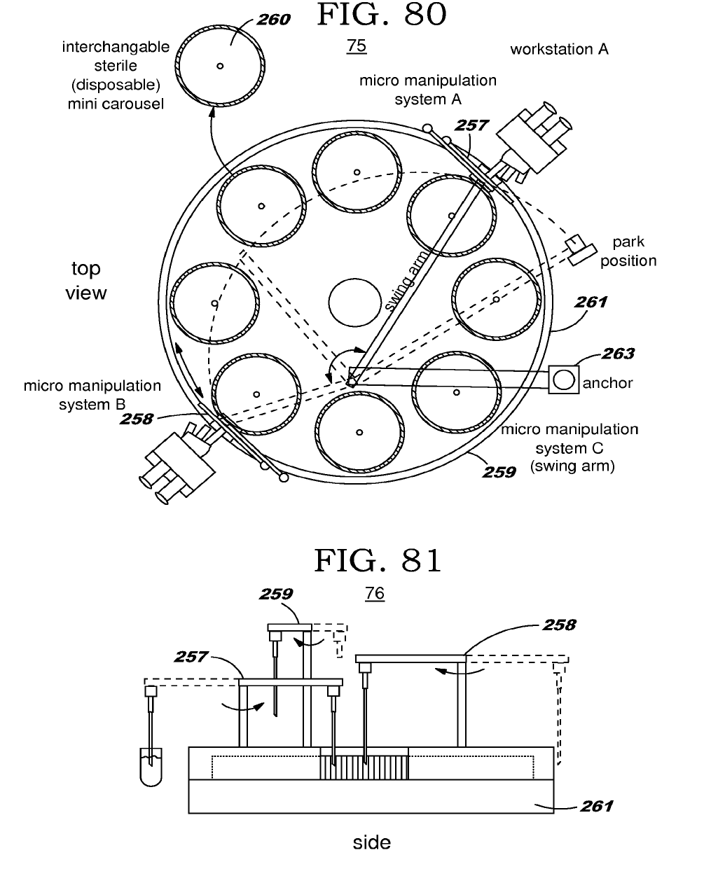

FIG. 80 is a top plan view of a micro-manipulator workstation.

FIG. 81 is a left side view of the micro-manipulator workstation of FIG. 80.

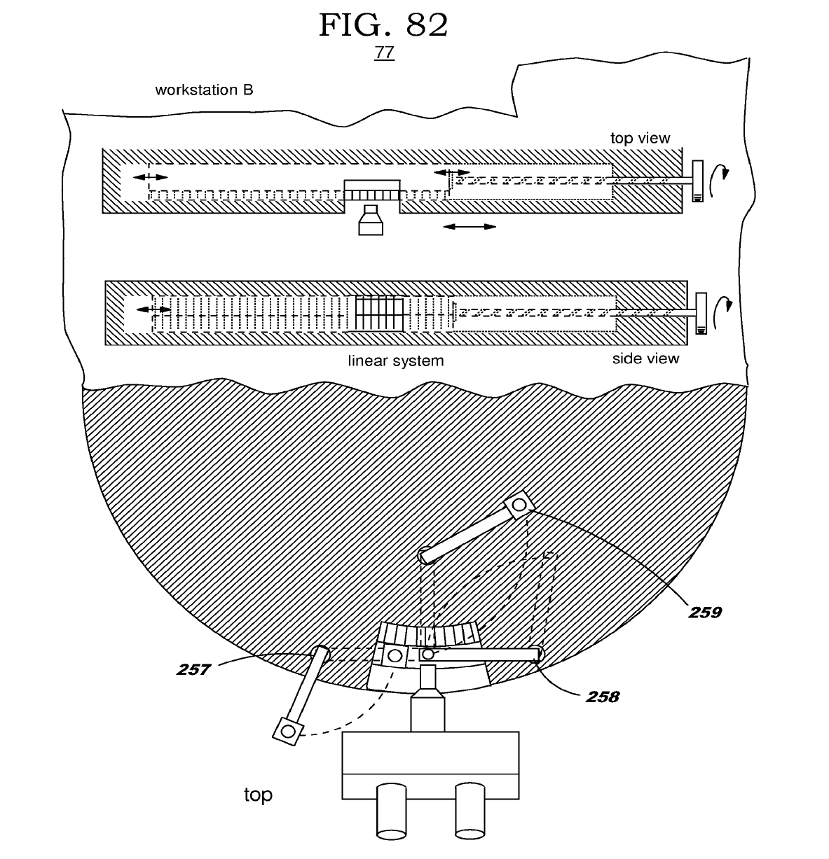

FIG. 82 shows alternate views of the micro-manipulator workstation of FIG. 80.

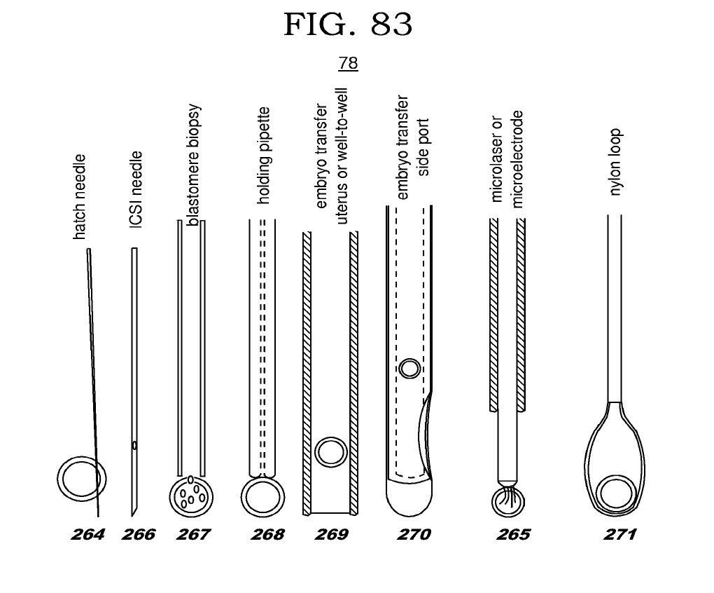

FIG. 83 shows examples of micro-tools.

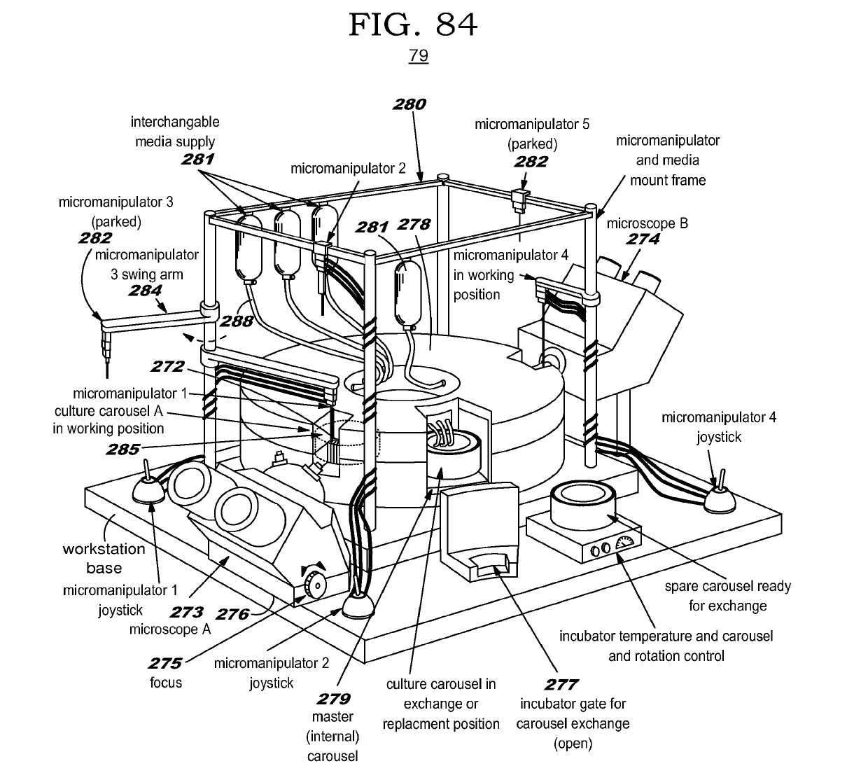

FIG. 84 is a perspective view of a prototype micro-manipulator workstation.

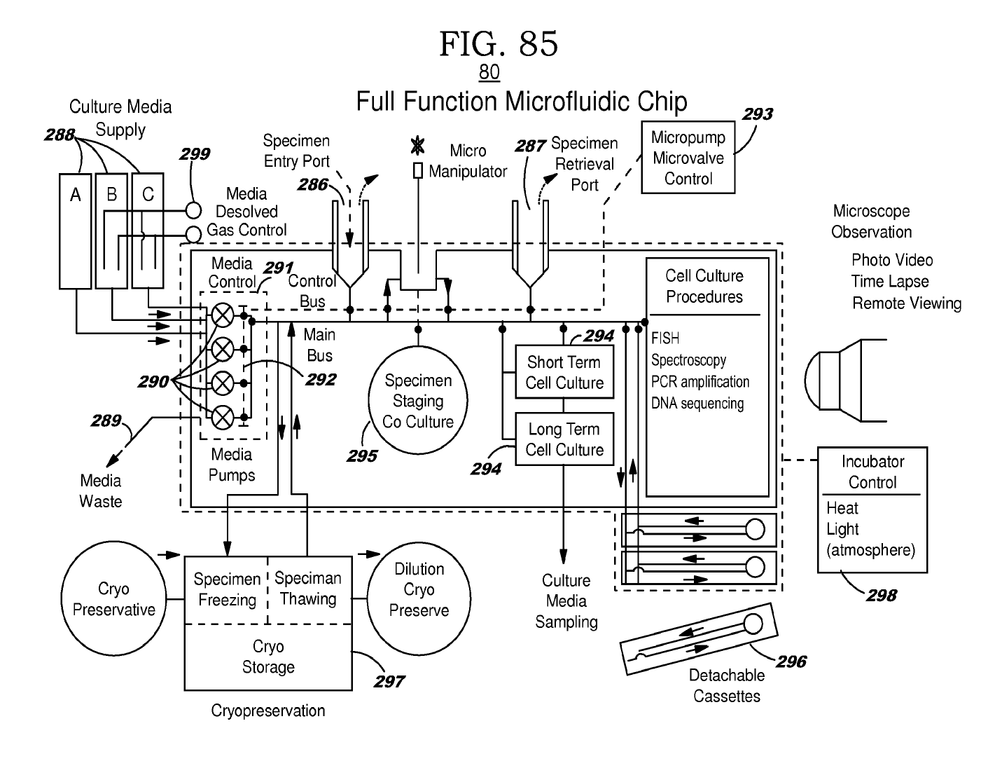

FIG. 85 is a schematic of a full function microfluidic chip.

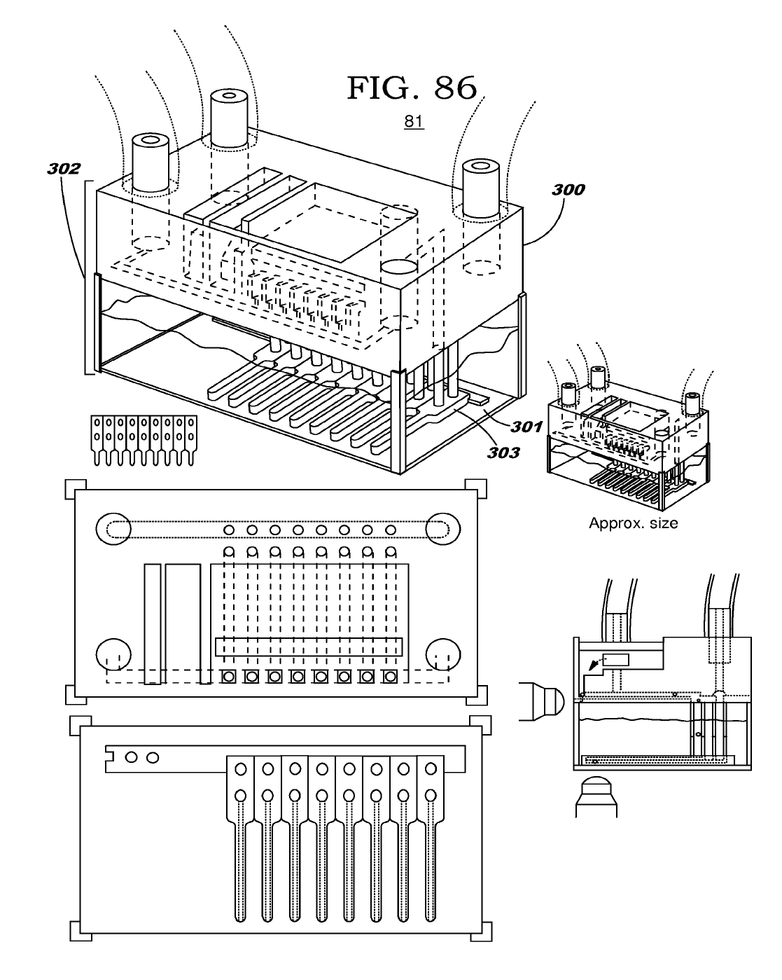

FIG. 86 shows a two-tiered full function microfluidic system.

### DETAILED DESCRIPTION

A more detailed description of components of a microfluidic IVF system is now provided.

The first component is a sperm separation system. The goal of the microfluidic sperm separation system is purification of sperm from semen and separation of normal sperm from those with chromosomal and morphological abnormalities is. If sufficient separation resolution is achieved by the system then simple inexpensive separation of X and Y chromosome sperm may be feasible, allowing sex determination of offspring in fertility patients and in commercial livestock.

A fractional distillation system permits exchange of sperm across laminar flow media streams along redundant parallel channels. Such a system may utilize either a passive gradient generator or an active gradient generator. The separation network is a “chicken-wire” configuration of adjacent, communicating laminar flow microchannels. Network gradient examples include albumin concentration gradients, chemotactic agents, pH gradients, sugar or carbohydrate gradients, and Percoll density gradients, or thermal, electric field, magnetic field, or centripetal force gradients.

Sperm cross the laminar flow boundaries in these channels in an asymmetric manner due to the slightly different concentration composition of the adjacent laminar flow streams. The basic components of a sperm separation system can be incorporated onto a single microfluidic chip, including the semen (or sperm solution) entry and exit ports, base media entry port, gradient solution entry ports, gradient generator, and the network feed channels along with the separation network and separation product exit ports.

Exemplary goals of the sperm separation system include:

1. Purify sperm from semen—Active sperm will cross from the primary laminar flow stream into the adjacent laminar flow stream using their self-powered motion, while semen fluid components and cellular debris remain in the primary stream.

2. Purify processed sperm samples—Pre-washed and processed sperm samples from cell pellet wash, semen dilution, swim-up, or density gradient techniques can be further purified by the microfluidic parallel network system.

3. Transfer active sperm into another fluid media without need to centrifuge into pellet (especially “fragile sperm” that would not survive high G-forces).

4. Separate sperm by their motility properties—sperm motility, velocity, and lateral velocity parameters occupy a wide spectrum. The most active sperm will have a much higher “cross section” of crossover into an adjacent fluid stream, and will separate themselves into a “motility gradient” in a microfluidic net.

5. Separate sperm by density. Sperm have a cellular density slightly higher than water (and seminal fluid). Greater separation by microfluidic net may occur if adjacent stream flow density is appreciably greater (or lesser) than sperm density. Physiologically better sperm tend to have an ideal density and can be purified on a centripetal density gradient with current techniques. A density gradient parallel fluid stream net may separate sperm by density without need for a centrifuge.

6. Separate X and Y chromosome sperm—The mass of X sperm is approximately 3 percent higher than Y sperm, moves slower than Y sperm (average long term velocity) and are longer lived. Current separation techniques are cumbersome, expensive, and relatively inefficient (eg flow cytometry, chromatography). A microfluidic net may be much less expensive and possibly more efficient, especially if media or force gradients are applied.

7. Separate sperm by their chemotactic responsibility—More responsive sperm will have a higher crossover rate into an adjacent fluid stream containing a chemotactic factor.

8. Separate by sperm mass, forward speed, lateral movement, or capacitation status.

Referring to FIG. 14, fluid flow in microfluidic channels has a very low Reynold's number and thus enforces a laminar flow. Adjacent laminar channels A, B in shared region do not mix, so primary sample channel containing active and inactive sperm and debris will flow through from A to A′. Very active swimming sperm on the interface of A and B laminar flow streams may cross into the B stream and exit at B′ as a purified active sperm sample. Nearly all debris and inactive sperm will exit at A′.

FIG. 15 addresses the reason this system is contemplated to exhibit a significant decrease in the number of sperm that serially progress over to subsequent streams. Most sperm samples contain a vast number of sperm (typically millions) and final sample sizes will probably have greatly reduced total number of sperm, but still remain functional for intrauterine insemination or especially ICSI, which require only a tiny fraction of the number of sperm in the initial sample.

Referring next to FIG. 16, further increase in system efficiency may be possible by implementing a “network” or “net” configuration to replenish the individual streams by periodically returning them to their source stream. The relative “degree of purification” should remain stable for each stream once equilibrium is achieved between the replenishment shared streams and the delivery shared streams.

Doubling the stream volume used in the separation or distillation process can be accomplished by duplicating the channel net on the opposite side of the raw sample channel as shown in FIG. 17. Efficiency may be further altered by increasing the flow rate through the system, pulsing the flow rate (stop and go or fast and slow) to allow more or less time for sperm to cross into adjacent laminar streams.

FIG. 18 provides examples of how alternating the channel size or geometry may be used to change the flow characteristics and sperm crossover characteristics of the system. The B channel may have a larger or smaller width or diameter from the A channel throughout its length, or stepwise with sequential shared channels.

Refinement in sperm separation, purity, and efficiency may be applied to the system in the form of gradients in forces, temperature, fluid density, flow speed, fluid velocity, or chemotactic capacitance factors along the linked parallel channels or across the channels. FIG. 19 demonstrates gradient across channels. FIG. 20 demonstrates gradient along channels.

FIG. 21 demonstrates a temperature gradient.

FIG. 22 demonstrates a flow velocity gradient.

Referring next to FIGS. 23 and 24, sperm tend to swim directly into the moving stream direction, and their average velocity can be enhanced by centrifugal force in their forward direction or reduced by centrifugal force in the opposite direction. This may be of particular use if channel fluid velocity remains constant from pump pressure in the microchannel while sperm forward velocity is markedly reduced by an opposing G force, resulting in relatively exaggerated lateral motion. Enhanced sperm separation may then occur at the parallel laminar flow stream interface. An off-axis G force may be useful to enhance stream interface crossover in a preferred direction. Referring to FIG. 25, a microfluidic chip 55 is placed in a centrifuge 56, which is used to generate a “G force” to enhance or counteract microfluidic fluid flow, or to generate a “cross force” to fluid flow perpendicular to or at an angle to fluid flow. For sperm separation applications, the additional force vector is used to advance or retard sperm velocity in the microfluid stream, or to increase average sperm velocity in an off-axis direction.

A flat unidirectional laminar flow microchannel net is limited in sperm purification time and exchange steps by the linear distance from the beginning to the end of the next channel. Because sperm are so numerous, the proportion crossing into the purification channel may be very small after one pass through the length of the net. FIG. 26 demonstrates a looped laminar flow channel system. Sample 57 makes multiple passes through the through the series of microchannels 58, which increases the amount of time and the eventual flux of sperm into the purification streams. Micropumps 59 keep sample 57 moving. The purification channels can also re-circulate through the net to collect a larger number of sperm at equilibrium. Referring next to FIG. 27, side channels 60 for injection of raw sample, replenishment of channel media, removal of wastes, and recovery of final purified sample can be added to any or all microchannels 58. As shown in FIG. 28, manufacture of the system can be simplified by fusing two or more plates 61, 62 with active channels engraved in each, for instance the net 63 in one plate 61 and the entry 64 and extraction 65 channels in the other plate 62. A cylindrical net 63 such as that shown in FIG. 29 incorporating micropumps, entry 64, and extraction 65 channels can run for long periods continuously.

Depending upon the sperm separation requirements for specific applications, various types of microfluidic gradients can be incorporated into the fractional distillation net configuration, including: (1) a fluid density gradient, e.g. a low to high Percoll concentration; (2) a fluid viscosity gradient, e.g. a low to high Albumin concentration; (3) a chemical gradient, e.g. electrolyte, calcium or potassium, etc; (4) a chemotactic gradient e.g. oocyte co-culture fluid; or (5) an osmotic gradient e.g. solute or colloid.

FIG. 30 demonstrates how a gradient system would work. Gradient components are added to parallel channels A-G at successively increasing or decreasing concentrations.

Sequential enrichment of motile sperm 66 occurs at each microchannel 67 shared interface as shown in FIG. 31, with a distillation of preferred sperm into higher and higher concentrations with more distant parallel channels. For example, big sperm preferentially attracted across shared stream laminar interface into higher concentrated media.

Automated microchannel mixers are used to generate concentration gradients. Mixing nodules are required to break the laminar flow of the concentrate fluid and the media fluid into chaotic flow in order to mix the fluids into an intermediate concentration. Examples of mixing nodules are shown in FIG. 32. Pure concentrate 68 is placed in microchannel 70. Pure media 69 is inserted at opening 75 and enters microchannels 71, 72, 73 and 74. Mixing nodules 76, 77 and 78 are placed at microchannel intersections. A, B and C show sample mixing nodules. Mixing nodules A and B are static, mixing nodule C is dynamic with a micromachine rotating vane wheel 79 powered by off-axis input fluid flow. Multi (micro) channel output of the gradient generator is fed into the sperm separation net configuration where laminar flow maintains the fixed concentrations in each channel.

FIG. 33 shows an alternate single chamber mixing nodule. A large chamber 80 receives a high concentration input channel 81 and a zero concentration input channel 82. The two input channels mix with a random or patterned barrier array to generate chaotic flow, then exit into multiple parallel microchannels 83, 84, 85, 86, 87 of increasing concentration to restore laminar flow. The parallel micro-channels are then used to weave the distillation net for sperm separation.

FIG. 34 demonstrates the basic components of a sperm separation system incorporated onto a single microfluidic chip 88, including the semen (or sperm solution) entry port 89, sperm waste exit port 91, base media entry port 92, gradient solution entry ports 93, 94, and the network feed channels 95 along with the separation network 96 and separation product exit ports 90a-90j. Fluid flow along the chip begins at the base media 92 and gradient solution ports 93, 94, with these fluids mixed in a continuous concentration spectrum by the passive gradient generator 97. The gradient spectrum is broken up into discrete ascending (or descending) solution concentration feed channels 95 running in parallel from the gradient generator 97 to the separation network 96, reestablishing laminar flow. The separation network 96 acts as a fractional distillation system that permits exchange of sperm across laminar flow media streams along redundant parallel channels 95. Sperm cross the laminar flow boundaries in these channels 95 in an asymmetric manner due to the slightly different concentration composition of the adjacent laminar flow streams. The asymmetrical crossing of sperm across streams results from the interaction of two factors: (1) the composition and concentration gradient of the media solution and (2) the size, shape, motility, and other morphological and movement characteristics of the sperm.

The microfluidic chip and all components thereof may be made using soft lithography plastic, Polymethylmethacrylate (PMMA), glass or DMSA. One skilled in the art will understand the benefits and drawbacks of each of these materials.

The slight behavioral differences in sperm activity in the different stream solutions determines which of the adjacent streams the sperm “prefers.” For example, a smaller faster Y chromosome sperm may be able to more easily penetrate into a higher concentrated albumin solution stream than a larger, slower X chromosome sperm which may “bounce off” the concentrated albumin solution laminar “wall.” Even tiny asymmetries in separation behavior are multiplied by the fractional distillation nature of the separation web, with each solution concentration stream respectively and alternatively exposed to the adjacent higher and lower concentration stream. A sperm with asymmetric preference for one concentration solution will slowly work its way over to the most favorable stream, and is eventually collected with a cohort of “like-minded” sperm at the final exit port. Very similar sperm are shuffled and concentrated into the stream with optimal favorable concentration solution.

Sperm activity and morphology parameters that may influence functional separation include multi-sperm adhesion and clumping, sperm mass, sperm velocity and forward progression, and head shape. The difference between monosomy and trisomy sperm mass exceeds the difference between X and Y bearing sperm, so a mass separation network may require a collection of many fractions at the end of the chip to obtain the purified sperm type (useful for sex selection and for avoiding fertilization by abnormal sperm.)

FIG. 35 shows other net configurations which may suffice to maintain the fractional distillation function of the microfluidic sperm separation system, ranging from widely separated “chicken wire” laminar channels 98 to narrow alternating parallel flow “vanes” capable of maintaining separate laminar flow streams 99.

In order to increase the volume and speed of sperm samples through the separation process, two or more separation chips can be operated simultaneously in parallel fashion, as shown in FIG. 36. The most efficient system stacks chips 100 with each layer independently operating as a separation network, but sharing sample 101, fluid 102, and recovery 103 ports.

Certain sperm separation applications may require external gradients or forces applied to the fractional distillation network. FIG. 37 shows, a temperature gradient can be applied across or along the separation chip 104 axis for thermotactic separation, with 105 being a higher temperature and 106 being a lower temperature. An electric field 107 or magnetic field 108 may be applied for electromagnetic separation, especially if ferrous micro-beads are attached to sperm or other cells or proteins as part of their separation identity. Visible, ultraviolet, or infrared light 109 can be applied for photon sensitive separation procedures, and centrifugation 110 of the entire chip can be used to apply G-forces along or across the axis of the separation chips in order to change the sperm velocity vectors.

FIG. 38 shows other variations in sperm separation chip design, including serial application of raw sample streams using two or more linear insertion 111, 112 and extraction 113, 114 ports, each pair for individual raw samples, along the separation network central corridor, or recirculation of the raw specimen multiple times through the specimen channel 115. This configuration would be useful to maximize the extraction of motile sperm from a prolific raw semen sample.

The second component of a microfluidic IVF system is a vertical micromanipulator.

Currently available cell culture micromanipulation is done using a relatively large cell holding pipette and a separate smaller micropipette or tool immersed in a Petri dish, each with its own micromanipulator actuator. The suction holding pipette is kept stationary during the manipulation procedure, but is required due to Petri dish geometry limitations. This classic system is required because oocytes and embryos are cultured in Petri dishes, freely mobile in a relatively immense volume of culture media fluid, and observed typically by an inverted microscope. The proposed innovation replaces the holding pipette with a stationary microfluidic suction channel, eliminating the requirement for one of the micromanipulator actuators. The micropipette tool is operated by a single actuator, simplifying the system and reducing instrument costs. A vertical orientation of the micromanipulation tool allows full access to the biological specimen when immersed in cell culture media.

As shown in FIG. 39, the function of the vertical micromanipulation system duplicates the classic system with the advantage of reducing the micro-actuators to a single tool manipulator by replacing the holding pipette with a suction microchannel 116 built into the microfluidic chip 117, or by trapping the specimen with micro-well geometry. The complexity, labor effort, and cost of the system is significantly reduced without sacrificing utility or versatility. Because the specimen 118 must be submerged continuously in liquid media 119, and the micro-actuator generally operates in air, the simplest configuration of the microfluidic system is holding the specimen 118 on a horizontal surface by a suction port microchannel 116 beneath, with the micromanipulation tool 120 descending from the atmosphere above through the surface of the liquid media 119 to approach and contact the top of the specimen 118. The micromanipulator 120 is in a vertical position held above the specimen 118. The micromanipulator 120 is difficult to see through an inverted microscope 121 because from the inverted perspective the micromanipulator 120 is hidden behind the specimen 118. For most applications, better visualization of the micro-tool operations is from the side, so a side mounted microscope 122 or mirror system (typically 45 degree mounted) for an inverted microscope is preferred. A side mounted microscope 122 with a vertical micromanipulator 120 and a specimen 118 held on a horizontal surface by a suction port 116 beneath duplicates the classic system almost perfectly except the working frame is rotated 90 degrees. Micromanipulation by a trained classic system operator requires little or no retraining, especially if the microscope objective or video monitor is rotated 90 degrees to match the classic visual orientation.

Microfluidic chip 117 may be made using soft lithography plastic, Polymethylmethacrylate (PMMA), glass or DMSA. One skilled in the art will understand the benefits and drawbacks of each of these materials.

Specimens 118, can be held and manipulated in individual wells 123, or can be operated upon as a group along a row of suction micro-ports 116 in a group well 124, an especially useful configuration for repetitive parallel applications. A row or array of micro-ports on the operating horizontal micro-well surface can be used to position and move oocytes and embryo specimens by sequential or programmed micro-port suction patterns. Specimens can be moved along the array to culture, holding, viewing, micro-manipulation, staging, or recovery positions by sequentially alternating suction and reverse flow through the holding ports. For most applications, the microfluidic chip and micro-wells are comprised of transparent material to allow visualization through microscopes.

Other angles can be used for special system requirements. For visualization through an inverted microscope, the specimen 118 can be held on a vertical wall by a horizontal oriented suction port 125, and the micromanipulator 120 approaches from the side by an angled actuator or tool mount. Alternately, the chip can be tilted to various angles as long as the specimen 118 remains under the media 119 surface and the micro-actuator 126 remains above the media 119 surface.

Micromanipulation tools include interchangeable micro-needles, pipettes, catheters, wire or nylon loops, electrodes, micro-lasers, or any other useful micro item. Two or more tools can be mounted simultaneously on a single micro-actuator 127 at parallel or offset angles, and two or more tools can be used sequentially or simultaneously on a single specimen if they are mounted on separate micro-actuators 126a, 126b. Micro tools can be used for insertion, removal, or transfer of specimens, oocyte stripping, zona hatching, ICSI, blastomere biopsy, specimen injection of DNA, RNA, protein, or dye solution, catheter loading, and specimen rotation among many other procedures. Many of these procedures can be automated or performed remotely by a programmed system or operator connected by internet, video and micro-robotic data stream.

For shorter term culture, open wells containing buffered media or media under oil layer are typically used, or the entire chip remains in a larger bath of media. The microfluidic media, vacuum control, and specimen insertion/removal interface ports with the macro world require capping or sealing between micromanipulation procedures, or when the chip is detached from the fluid and control systems for transport or cryopreservation storage. Cap and seal methods include heat seal 128, hard cap 129, or Silastic membrane covers 130 for needle penetration.

In vitro fertilization laboratories use current micro-manipulation technology for several basic procedures, including: inter-cytoplasmic sperm injection; embryo blastomere biopsy for preimplantation genetic diagnosis; polar body biopsy for preimplantation genetic diagnosis; removal of fragmentation debris from embryos prior to uterine transfer; assisted zone hatching; micro-injection of DNA, RNA, or tracking dye solutions into specimens; and microinjection of cryopreservatives into oocytes.

The vertical micromanipulator described above can be utilized for all of the above basic procedures, and can also be used for: oocyte cumulus stripping; micropipette catheter loading; and micromanipulation injection of florescent in situ hybridization material (FISH).

The vertical micromanipulator can also be applied to other types of cultured cells and tissues for: microelectrode insertion into cells or tissue; micropipette electrode probe; micropipette injection of cytoplasm components, or for nuclear or organelle transfer.

FIG. 40 demonstrates a microfluidic oocyte stripping method: The oocyte 131 with cumulus mass 132 is inserted into the chip well 133 and drawn through a funnel 134 into the specimen microchannel 135. Fluid is aspirated out the end 136 of the specimen microchannel until the oocyte 131 arrives at position A, then fluid flow is stopped. With oocyte 131 at position A, media fluid containing cumulus digestive enzymes (typically hyanourandase) is pumped rapidly and alternatively back-and-forth along stripping channel 137 to remove the cumulus mass 132 from the right side of the oocyte 131, with the detached cumulus fragments disposed of through the stripping channel 137. The stripping channel flow is then stopped, and slow aspiration of fluid from the end 136 of the specimen microchannel 135 is used to move the oocyte until it arrives at position B. The rapid alternating fluid flow procedure in the stripping channel 137 is repeated until the cumulus mass 132 is removed from the left side of the oocyte 131. Injection of fluid into the specimen microchannel is then used to move the oocyte 131 back to its starting position where, if needed, it is rotated by additional flow to orient any remaining cumulus attachments toward the specimen channel so the entire process can be repeated until complete stripping is achieved.

Note: The stripping channel is too narrow for the oocyte to pass through.

Turning to FIG. 41, alternate stripping configurations may employ a curved specimen channel 138 to apply physical bending stress to the cumulus mass for easy removal, two stripping channels 137a, 137b used to simultaneously remove cumulus from both sides of the embryo, or combination of both.

The third component of a microfluidic IVF system is microfluidic cassette cell/tissue culture system. Currently available microfluidic cell culture systems utilize a single microchip for insertion, storage, manipulation, culture, and recovery of numerous tissue fragments or cells. These microchips incur the same cost, capacity, and complexity whether they hold a single cell or hundreds of cells. The proposed innovation separates the microchannel and micro-chamber culture systems into individual, identical, and detachable units that are operated in parallel for each individual cell or tissue fragment. The number of cassette units can be increased or decreased for each culture run to accommodate the appropriate number of cells or tissue fragments, and cassette units can be provided with customized culture media concentrations and flow rates. A suction holding channel can be incorporated into each cassette to allow built-in, sequential vertical micro-manipulation along the row of cassettes.

Referring to FIG. 42, microfluidic cassettes 142 are comprised of small chips 139 with single or multiple cell culture chambers 140 and the associated microfluidic channels, valves, pumps, and ports, and contain specimens 141, such as cells, tissue fragments, gametes, embryos, and explants. The cassette 142 is designed to hold, store, cell culture, stage, manipulate, freeze (cryopreserve), and thaw these specimens 141. The cassettes 142 can be designed as independent units performing all functions, or small detachable units 143 for specific separate functions or applications (e.g. a detachable chip for cryopreservation freezing of a cell, leaving the cohort of other cells attached to the culture system for continued culture).

The cassettes are typically made of transparent material, such as glass, plastic, Polymethylmethacrylate (PMMA) or DMSA to allow observation of cultured cells by a top view, side view, or inverted view microscope. Multiple simultaneous views can be provided by small mirrors (typically mounted at 45 degree angles) mounted on the microscope, cassette, or independently—an arrangement which is especially useful for viewing complex specimens or for 3-D guidance of micromanipulation tools. For ease of viewing multiple specimens simultaneously, or several specimens in quick succession, the cassettes can be aligned and configured in rows, tiers, or clusters.

Arrays or rows of cassettes 142 can be viewed (and associated specimens operated upon) in succession by placing them on moving racks, conveyors, or carousels 144, or left in position and alternately moving the microscope 145. The active viewing region defines a micro workstation where specimens can be successfully observed, photographed, and micromanipulated. Work station procedures include observation of specimens from remote locations or at odd hours via a video and carousel/rack control link. Automated photo or video recording of specimens can be accomplished by programmed micro processor control of cameras and rack movements. An example of this system would be time lapse video photography of embryo development or cell layer growth response to a change in culture media. Movement of specimens between cassettes or other microfluidic chips can be automated or performed remotely by linked operator.

Control of culture media flow to the specimen is required to deliver nutrients and remove wastes. Media can be delivered to specimens held in microchannels, microchambers, fluid traps, or on suction ports via microchannels 146, typically two or more convergent upon the specimen site. Fluid flow can be continuous or pulsed, and is reversible to deliver or remove the specimen (or static if a relatively large volume of media is used.).

Insertion and removal of a specimen into or out of the cassette, and interface of the macroworld fluid and vacuum control lines, requires chip ports that can be “opened” and “closed.” A closed chip 139 contains ports that have hard caps 147, heat or adhesive sealable tubing 149, microvalves 150, or membranes 148 that can be penetrated by microneedles and pipettes. An open chip 139 is submerged in media and can draw or expel media from the external pool, typically via separately controlled port tube.

A variety of well shapes can accommodate various embryos or culture requirements. Simple low-cost systems can utilize cubic or rectangular prism or cylindrical wells, with or without a holding vacuum channel for micromanipulation stability. Alternate method for holding stability is via conical or pyramid well bottom to trap a spherical embryo during vertical micromanipulation. Side relief feature can be added to enhance the last step of mechanical assisted hatching.

FIG. 43 shows a number of well shapes, including a cylinder prism 151 with a vacuum channel 152; elevated 153 with a vacuum channel 154; prism 155 with side relief 156 and vacuum channel 157; conical pyramid 158 with vacuum channel 159; conical pyramid 160 with side relief 161 and vacuum channel 162; cylinder prism 163; conical pyramid 164; inverted dome 165; inverted cone 166; conical pyramid 167 with side relief 168.

Turning to FIG. 44, transparent culture wells 169 are aligned in rows for easy access and viewing by inverted, standard, or side approach microscopes. The size and shape of the wells are designed to fit the cell culture, culture media, and micromanipulation requirements. Well size ranges from slightly larger than an oocyte (approximately 80 μm mouse, approximately 120 to 150 μm human), to very large size depending on the required culture media volume. Slow flow or periodically changed or renewed media allows very small well volumes. Individualized or customized media requirements for individual embryos or cultured cells are best supplied to individual wells (i.e. one cell per each embryo) but grouped embryos or co-culture embryos may share larger wells or adjacent wells with common shared media.

Deep wells, such as those shown in FIG. 45, permit large media volume and overflow protection.

Microfluidic embryo hatching and loading into the embryo transfer catheter can be done using the microfluidic cell culture cassette system described above. Embryo hatching is done using the vertical micromanipulator, and embryo loading is accomplished by direct delivery of the embryo to the embryo transfer catheter via a microchannel, insertion of an intra-transfer catheter into the open access port or micromanipulation port on the cassette chip, or by extracting the embryo from the open access port on the chip using a pipette.

The fourth component of a microfluidic IVF system is a culture media supply to the microfluidic system.

Closed microfluidic embryo cultures systems have the advantage (over open well systems) of trapping culture media in channels and chambers without gas/fluid interface. Potential evaporation of media with associated solute concentration cannot occur, and escape or entry of dissolved gasses (nitrogen, oxygen, and carbon dioxide in particular) is minimal or absent. The need to expose culture dishes to an incubation atmosphere for several hours to equilibrate gas and temperature is eliminated. Rapid culture setup with immediately available pre-equilibrated culture media is a significant advantage of closed microfluidic systems. In addition, the requirement of very minimal culture media volumes (even for extended cultures) due to the tiny volumes of microchannels and microchambers is a distinct advantage for cultures using expensive media.

In order to supply microfluidic systems with appropriate culture media, a system is needed to pre-equilibrate media with the customized dissolved gas concentrations required for the specific application. Turning to FIG. 46, a relatively small volume system can be designed using an individual cartridge 171 of media 172 containing single, double, or triple (or more) gas “bubblers” 173 similar to an air bubbler system for fish tanks. Very rapid dissolved gas equilibration is achieved, and can be controlled by feedback from dissolved gas sensors 174 imbedded in the media or in microchannels and chambers fed by media lines. Individual nitrogen, oxygen, carbon dioxide, etc. gas concentrations can be separately controlled by individualized gas sensor feedback, and excessively high concentrations can be reduced by flushing with low or zero concentration carrier gas. Alternately, all gases can be premixed at the desired ratios, then delivered to a single bubbler line in the media cartridge, with concentration feedback adjustments applied to the premixing manifold. A media port 175 enables addition of media at any time. A gas exhaust port 176 maintains the pressure in cartridge 171. A bottom feed port 177 carries media 172 to culture chambers (not shown). Media port 175 and bottom feed port 177 can be reversed so that port 175 is used as a feed port and port 177 is used as a media port.

Turning to FIG. 47, preheating of media 172 can be accomplished with heating blocks 178, or by heating media after entry into the microfluid block. Media flow can be accomplished by micropump or syringe, or by gravity 179 with flow rate controlled by cartridge suspension height above culture block. Ambient atmospheric pressure needed for gravity flow is provided by an open filtered port on the cartridge. A trap system 180 will prevent the culture block from going dry in case the media supply accidentally runs out, incorporating a safety measure. A filter at the cartridge outlet can be used to sterilize the media by removal of microorganisms, and can remove stray gas bubbles before media is fed into microfluidic channels.

Turning to FIG. 48, an alternate system for fixed pre-established media and dissolved gas concentration can be supplied by a sealed container 181 with pre-equilibrated components.

The fifth component of a microfluidic IVF system is an intra-vaginal incubation module.

A version of microfluidic embryo culture incubation can be used to greatly simplify the in vitro fertilization process, and eliminate the standard in vitro fertilization incubation procedures and associated high cost of incubation equipment. Standard in vitro fertilization incubation steps include fertilization of oocytes by incubating them with sperm after oocyte capture and stripping, or incubating ICSI fertilized oocytes in large volumes of media in Petrie dishes or test tubes. These dishes or test tubes must be pre-equilibrated prior to insertion of oocytes, sperm, or embryos by keeping then in a standard cell culture incubator for 2 to 3 hours in order to stabilize the media fluid temperature and dissolved gas concentrations. After transferring embryos into the pre-equilibrated media, the Petrie dish or test tube containers are kept in the standard laboratory incubators for 1 to 6 days, after which the developed embryos are removed from the dishes and either transferred into the patient's uterus, frozen for delayed transfer, or (if development fails) discarded. Typically, the embryos are removed from the incubator once a day and inspected by microscope to monitor development, but these daily inspections are optional. The current in vitro fertilization process involves purchase, maintenance, and operation of large cell culture incubators along with their associated multiple gas lines, gas manifolds, and large compressed gas cylinders. In addition to large capital expenditure for this equipment, significant ongoing expense is involved with quality control and with constant operation and replacement of spent gas cylinders.

This process can be significantly simplified by an inexpensive innovation using intra-vaginal microfluidic modules. Turning to FIG. 49, after oocyte stripping procedures and ICSI or standard fertilization, the embryos are inserted into a small microfluidic chip 182 comprised of at least a media fluid entry port and embryo entry and exit port 183, an exit port 184, fluid chamber 185, and return channel 186. Optionally, the chip may contain a culture well or fluid trap (not shown). A second embodiment comprises a chip 187 with culture chambers 188. A micropump 191 powered by battery 190 pushes media into a chamber 193 where a piston 192 pushes the media through a feed channel 189 into culture chambers 188 and through a return channel 194 back to micropump 191.

Microfluidic chip 182 may be made using plastic, Polymethylmethacrylate (PMMA), glass or any material having similar qualities. One skilled in the art will understand the benefits and drawbacks of each of these materials.

Referring to FIG. 50 chip 182 (or 187) is encased and sealed inside a small, smooth, inert module 195. A clip 196 secures module 195, forming assemble capsule 197. Capsule 197 is then placed in the back of the patient's vagina, and held in place by a vaginal packing cloth or circumferential cervical ring for 1 to 6 days. The microfluidic chip contains enough liquid culture media to provide the embryo with sufficient nutrients and to dilute metabolic wastes for the entire incubation period. The intravaginal module is kept at body temperature with no ambient light during this time, and the media contains enough dissolved gas in the fluid volume to maintain physiologic oxygen and carbon dioxide concentrations for normal embryo development. At the end of the 1 to 6 day incubation period, the module is removed from the vagina, opened, and the microfluidic chip is retrieved and examined microscopically. Embryos with normal development are removed from the chip and either immediately transferred into the patient's uterus, or frozen for later thaw and delayed uterine transfer.

The incubation microfluidic chip and intravaginal module replace the current expensive and tedious laboratory incubation system, dramatically decreasing the cost of in vitro fertilization. In addition, the patient becomes more intimately involved with her fertility care, essentially acting as the embryo incubator. The microfluidic chip and/or module can be single-use disposable items, or reusable items after cleaning and resterilization. The basic design of the chip requires at minimum an entry exit port(s), method to seal media and embryos inside, no gas fluid interface for media (micro-channels and chambers are completely full), and sufficient volume of media to maintain nutrition and dilute metabolic wastes for the entire incubation period. The intravaginal module must be small enough to comfortably reside in the back of the vagina for several days, robust enough to withstand expected movement in its environment, sealed tightly enough to protect the enclosed chip from microorganisms and vaginal fluid contaminants, and be comprised of an inert, non-irritating surface material.

The advantages of microfluidic technology can be incorporated into the design of the chip. A passive chip is comprised of a sufficiently large media chamber to provide nutrient requirements for the embryos. A more advanced chip can include embryo wells extending from the media chamber for individual embryo containment, or incorporate a fluid trap or freezing stem, allowing rapid easy embryo freezing once the chip is retrieved. Microchannels, micro-chambers, embryo wells and fluid traps can be configured for more advanced functions, including continuous or intermittent circulation of media around the embryos during the incubation periods using a motion or battery powered micropump. A change in culture media at a specific time during incubation can be accomplished using a single media reservoir with a movable piston, or by keeping different types of media in two or more separate micro-reservoirs. FIG. 51 illustrates a chip 198 having culture chambers 199 containing embryos 200 covered in a primary media 201. A secondary media 202 is initially stored in a media reservoir 203. At a given time, piston 204 shifts through media reservoir 203, pushing secondary media 202 into culture chambers 199 and primary media 201 into media reservoir 203. Micropump or piston movement is either automated or accomplished manually (for instance movement of the piston by external magnet) at the time of temporary retrieval of the module part way through the incubation period.

The sixth component of a microfluidic IVF system is a microfluidic freezing stem.

This innovation increases the freeze/thaw survival of cells and tissues by increasing the freezing rate with reduction of the thermal momentum of the culture system. After insertion of cells into the microfluidic system, the specimens are trapped by media fluid flow in a narrow stem extending from the microchip. The thin-walled exposed stem permits very rapid freezing once the microchip is plunged into liquid nitrogen or similar cryogen. After thawing, the process is reversed to recover the biological specimen.

The purpose of the freezing stem is to maximize the rate of freezing of the oocyte, embryo, cell, or tissue fragment specimen by decreasing the mass and thermal momentum around the specimen and increasing the heat flux out of the specimen when it is placed into liquid, solid, or slushed cryogen. Turning to FIG. 52, a relatively small, thin stem 205 of the microfluidic chip holds the specimen 206 away from the larger mass of the chip body 207 in order to increase the exposure the specimen to rapid heat removal by the cryogen. The specimen 206 is contained in a microchannel 208 extending from the main mass of the chip 207 into the low mass and thin-walled stem 205, and held near the tip of the stem 205 to be exposed on nearly all sides to cryogen, and is protected from direct exposure to the cryogen to prevent contamination by microorganisms, toxins, or debris. Relatively toxic but more efficient cryogens, such as liquid propane (cooled by liquid N₂), can be used for rapid freezing or vitrification of the specimen, enabled by the physical barrier of the freezing stem chip design. The other “workings” of the microfluidic chip, including the necessarily larger insertion and removal ports or fluid entry/exit ports, connectors, and microvalves and sorting channels are kept away from the stem because of the relatively high mass and thermal momentum. The freezing stem also provides a physical barrier between the specimen and the cryogen to prevent contamination, and can double is a microfluidic cell culture chamber and a micromanipulation platform.

General operation of the freezing stem is as follows. First, the specimen is immersed in a small amount of culture fluid or fluid droplet (with optional addition of cryoprotective solution). The specimen is then positioned at the tip of the stem. Optionally, a cell culture of the specimen may be taken before freezing. Turning to FIGS. 53 and 54, chip 207 is plunged; typically stem 205 first, into a liquid, slushed, or frozen cryogen 209. The stem and specimen are then stored at cryogenic temperatures. The specimen is thawed by rapidly plunging the freezing stem into a relatively large volume of warm water (liquid) bath or by exposure to radiant heat or microwaves. Cryoprotective solution and/or cell culture of the specimen are diluted post thaw. The specimen is retrieved from the stem as shown in FIG. 55. Media is aspirated into the stem 205 in the reverse direction that it originally entered, thus pushing specimen 206 out.

Chip 207, including stem 205 may be made using plastic, Polymethylmethacrylate (PMMA), glass or any material having similar qualities. One skilled in the art will understand the benefits and drawbacks of each of these materials.

Very high freezing and thawing rates are achieved by maximizing heat flow into and out of the specimen in the stem, using low mass (small stem size), low thermal momentum, high surface to volume ratio (long stems, hemispheric tip), and thin walls. In general, a larger “body” of the microfluidic chip attached to the stem is required to house the specimen insertion and retrieval operations and the microfluidic channels, ports, valves, and other interface systems. Increasing stem length holds the larger mass and thermal momentum body away from the specimen to increase the freezing rate, but also increases the physical fragility of the device.

The size of the microfluidic chip attached to the freezing stem depends upon the requirements of the system, but simple applications can use a relatively small total chip size. FIGS. 56 and 57 show an efficient configuration. A microfluidic chip body 207 contains enough size and mass to accommodate the specimen and fluid entry port 211 and exit port 212 and connectors 213 along with the associated microchannel 214 extending into a functional freezing stem 205.

When maintained at constant, appropriate temperature the freezing stem can double as a microfluidic cell culture system after placement of the specimen in the tip trap. A static culture method involves no active fluid medium flow to or from the specimen during the culture, but an active system involves either continuous-fluid flow of media or periodic flow (pulsed flow method) down the specimen channel and returned via the return channel. The active flow system allows sampling of the return media for research or clinical assays, and allows sequential changes in the culture medium composition to optimize cell culture conditions. Immediately prior to freezing the specimen, a stepwise or continuous increase in cryoprotective solution concentration as shown in FIG. 58, can be infused around the specimen at the trap position, and after thawing the process can be reversed by stepwise or continuous dilution of cryoprotective solution, followed by reversed flow to retrieve the specimen. Several freezing stems can be incorporated as a group to allow parallel culturing and simultaneous freezing and thawing of multiple specimens.

Turning to FIG. 59, the rate of freezing and thawing can be further increased by introducing a gas bubble 216 into the specimen channel 217 and advanced close to the specimen 206 in order to decrease the droplet size and associated thermal momentum at the tip of the stem 205.

As illustrated in FIG. 60, the confined geometry of the specimen trap 220 at the end of the freezing stem 205 can be used to hold the specimen 206 stationery for micromanipulation tools 222 inserted down the specimen channel 217. Alternately, the return channel 224 connection to the trap 220 can be configured as a suction holder to stabilize the specimen 206 for micromanipulation. Turning to FIG. 61, a safety cap 226 is used to cover the fragile stem 205 during culture, micro-manipulation, and storage between freezing and thawing.

Turning to FIGS. 62 and 63, the junction 227 at the tip of the freezing stem 205 acts as a fluid trap for the specimen 206, ensuring free movement of the specimen 206 along the larger diameter specimen channel 217 and the ability to hold the specimen 206 in a stationary position at the most thermally exposed part of the mechanism (the tip) for long term culture or for rapid freezing. Simple specimen traps involve a small connecting channel 228 or microscreen between the large diameter specimen channel 217 and the typically smaller diameter return fluid channel 224 located at the very tip of the freezing stem 205. The specimen 206 is too large to pass through the connecting channel 228 or screen, but fluid flows around the specimen 206 into the connecting channel 228 and on through the return channel 224, with a specimen 206 held against the terminal wall by fluid pressure.

FIG. 63 also illustrates examples of single and double return channel designs, and long and short stem lengths with associated stem cross-section and longitudinal sections. Typical actual sizes of cassettes are also illustrated.

The specimen is moved from the entry port (or micromanipulation or primary culture portion of the main body the chip) to the end of the freezing stem by fluid flow from the specimen channel port to the connecting channel and back through the return channel. The fluid flow is reversed after thawing the specimen in order to move the specimen from the tip of the freezing stem back to the entry/exit port. Typical specimen thaw is by rapid plunge into a relatively large volume of warm water bath or media bath. If cryopreservation solutions are required for some applications, the cryopreservation solution at appropriate concentration is delivered to the specimen by fluid flow through the specimen channel, with the advantage of slow, rapid, or stepwise changes in cryopreservative concentration as needed through the connecting ports, and with post thaw dilution of cryopreservation solution done in the same manner before reversed flow recovery of the specimen.

FIG. 64 illustrates a variation involving a multi-well chip 229 with associated rows of multiple freezing stems 230 extending from one or more edges of the chip can hold between two to 20 (or more) specimens 231, all to be simultaneously plunged into cryogen 209. This system allows batched freezing of cells, oocytes, and embryos, and individual specimens can be added (or removed) to the chip prior to freezing. This principle can be applied to rows or stacks of combined incubation and freezing stem units, connected to a parallel media flow system as illustrated in FIGS. 65 and 66. This arrangement allows individual detachment of specific freezing cassettes for cryogen plunge, leaving the attached cassettes for continued culturing or for extraction of specimens for disposal or transfer.

FIG. 67 shows examples of long stem 205 freezing cassette with two fluid ports 211, 212 in the body and a single specimen channel 217 with two return channels 224a, 224b, an over-design feature to provide backup in the event of obstruction of one return channel by specimen or debris. Cross and longitudinal stem sections are illustrated.

Turning to FIG. 68, another useful variation of the combined vertical micromanipulation, embryo incubation, cryopreservation microfluidic chip incorporates a freezing stem 205 extending from the side of the main chip body 207. This allows easy visual control of the micromanipulation procedure, movement of the embryo to the end of the freezing stem, inspection of embryo development during the incubation period, observation of the embryo during the cryopreservative concentration procedure, and control of placement of the pre-freezing gas bubble in the specimen channel, all through a side view microscope 122. These procedures can be viewed in a single microscopic field without requirement for moving the microscope or rotation of the microfluidic chip. By aligning the chips in a row or array in parallel fashion, the side arm design allows serial viewing of the working fields of multiple chips, with the added benefit of aligning the fluid ports on the main bodies of the chips for connection to a parallel media supply manifold.

Turning to FIG. 69, upon freezing, water-based culture media expands approximately 9% in volume with ice formation. In an entirely closed and sealed fluid filled chip, the ice expansion will crack the chip open. An inert gas bubble 232 (nitrogen, argon, etc.) will absorb the increased ice volume and prevent damage to the microfluidic chip 207.

Access ports on the main body of the freezing stem can be covered with a Silastic membrane to maintain a closed culture cell, but allow penetration of the access port by a metal or plastic needle. The needle can be used to supply culture media, insert or removed specimens, or in a special case can provide a channel for micromanipulation tool access to the specimen. After withdrawal of the needle, the defect in the Silastic membrane can be sealed with adhesive to provide further protection from leakage or from direct exposure of the specimens to the cryogen.

As illustrated in FIG. 70, a special variation of the freezing stem system involves no stem at all. Instead, the specimens 206 are placed in closed micro-chambers 233 in the interior region of very thin cassette chips 234 having entry and exit ports 235, 236. These specimens 206 are frozen by rapidly plunging in the entire cassette chip 234 edgewise into liquid or slushed cryogen 209, resulting in very rapid heat removal from the enclosed specimen chamber 233 through the top and bottom surfaces of the chip 234.

Cassette chip 234 may be made using plastic, Polymethylmethacrylate (PMMA), glass or any material having similar qualities. One skilled in the art will understand the benefits and drawbacks of each of these materials.

FIGS. 71 and 72 illustrate a number of variations of the cassette chip 234. Increased heat flux and more rapid freezing can be achieved by reducing the thickness of the chip 234 at the location of the micro-chamber 233 (thinning the walls of the chamber), or by placing the micro-chambers 233 along the edge of the chip 234. The thin-walled chip 234 is subjected to significant thermal stress forces during the period of very rapid cooling, and thick walled ribs 237 can be inserted between freezing chambers 233 in order to increase the physical strength of the chip 234 during the freezing process without compromising the heat flux from the thin walled specimen chambers 233.

The basic individual components of microfluidic cell/culture system include culture microchambers and associated culture media delivery channels, freezing stems, micromanipulation wells and platforms, cumulus stripping channels, microscopic observation regions, and in more complex systems a series of micro pumps and valves to transport specimens and fluid along the microfluidic chip. An important part of any microfluidic cell culture system is the interface with the “macroworld”—the means in which fluid (and gas or vacuum) lines are connected to the chip, and the means in which samples are inserted into and removed from the chip. The fluid and gas lines from the macroworld are typically in the millimeter dimension scale and must be connected to the microfluidic channels which are typically on the micrometer scale, a scale change of 2 to 3 orders of magnitude. Likewise, specimens are transported in the macroworld using millimeter scale pipettes and vials, and must be transferred to and from microfluidic channels in the micron scale. In general, moving fluids and specimens between the macro and microworlds is accomplished via ports and wells on the surface of the microfluidic chip that funnel millimeter scale channels into micrometer scale channels. For example, oocytes and embryos are approximately 100 μm diameter and are transferred in 250 μm pipettes into 500 μm ports or wells, then are funneled into 150 μm microchannels. A 1 mm diameter fluid line connects to a chip port which funnels fluid into a 30 μm microchannel.

FIG. 73 illustrates a combined microfluid chip 238 with a specimen insertion/removal well 239, a stripping chamber 240, a micromanipulation well 241. Chip 238 can be used to inspect oocytes, then strip them of cumulus cells, then hold them in place for ICSI fertilization then culture them for several days during embryo development, then trap the embryos in a freezing stem 242. After adding concentrated cryoprotectant the chip is then discontinued from the fluid lines and plunged into a cryogen, and the frozen sample is stored. To thaw, the chip is placed into a warm fluid bath or microwaved, the lines are reconnected, the cryoprotectant is diluted, and the specimen is recovered. An alternate recovery method is physically breaking off the freezing stem after immersing it in a media bath. Alternately, the same four components can be located on individual chips 243, 244, 245 and 246 and connected by microchannel 247.

Microfluidic chip 238 may be made using plastic, Polymethylmethacrylate (PMMA), glass or any material having similar qualities. One skilled in the art will understand the benefits and drawbacks of each of these materials.

FIG. 74 illustrates the many ways a single microfluid chip can be used.

Linear row or carousel incubation wells may be filled with premixed, gassed, and warmed media under an oil layer for short-term applications. Serial short-term applications with intermediate media change requirements can be accomplished by the same system by moving individual embryos between wells using the micromanipulator to pull the embryo up from one well in a micropipette, then rotating the new well into the active position, and lowering the embryo into the new well. Longer term applications often require changing media on an intermittent basis, and this can be accomplished by feeding individualized media through microchannels into individual media wells, with a micro-valve control system arranged to deliver the proper media to the proper well, and remove media individually as test samples or waste. In a rotating carousel system, flexible tubing can be used to deliver various medias to appropriate microchannel ports on the carousel. Rotation of the carousel can be limited to a specific angle each direction to prevent over winding or entanglement of the media feed tubes.

FIGS. 75 and 76 illustrate a simple media feed system consisting of several media feed lines 248a, 248b, 248c, 248d connecting media tanks to microchannel entry ports on the inner circumference of a carousel 249. The carousel 249 rotates 180 degrees each direction from a parked position, allowing microscopic viewing access to the entire carousel circumference (all culture wells) without over winding, entanglement, or stretching of media lines. Micro-pumps can supply media feed pressure into the system, or a failsafe gravity feed can be used for critical applications, with pressure and flow controlled by elevation changes in the media tanks.

Four media lines are illustrated as a typical application, but as few as zero lines to a large number of lines (up to or even exceeding the total number of culture wells) may be employed as indicated by the application requirements. Other lines may include waste lines, connecting lines to wells across the carousel, or lines connecting to the other carousels. Electrical, power, and data wires may also be added in a similar non-entanglement arrangements above, within or below the carousel, including vacuum or other actuator lines controlling the microfluidic micro-valves inside the carousel.

FIGS. 77-79 illustrate a robotic microfluidic incubator system. The system consists of an upper heating unit (incubator) 250, a lower heating unit (incubator) 251, a carousel 252, a carousel rotation axis 253, multiple feed and exhaust lines 254, a microscope access slot 255 and a micromanipulator 256.

The thin transparent sidewall and close proximity of the embryo/oocyte/cultured cells to the sidewall allow close approach of a side view microscope with adequate focal length for mid to high power. This arrangement permits microscopic examination of multiple culture wells when arranged in rows (linear or along the circumference of a carousel). Manual or automated side to side movement of the linear well row, or rotation of the carousel, allows rapid inspection of the contents each well. Automated systems with video capability also allow remote inspection of wells by video connection or Internet connection, and automated video systems can record off-hours inspections or time lapse development in culture (i.e. embryo cell division progression, or axon growth in neuron cell cultures).

Cell culture requires stable, well controlled incubation temperatures, media control, and dissolved gas concentrations, along with minimal or controlled ambient light levels. A relatively compact incubation system can be designed around the linear well or carousel system to maintain constant temperature, light levels, and media and dissolved gas levels. For a carousel system a basic incubator design consists of an enveloping hollow cylindrical jacket containing a temperature control system, carousel rotation and well position control, low interior light levels, and media feed lines and waste lines. An access port is cut into one side of the incubator jacket to permit close approach of the side view (or inverted) microscope, and of the micromanipulator tools. The access port can be perpetually open, or can have a hinged door or gate which is closed between viewing sessions. Incubation jacket design for temperature control consists of an insulated high thermal momentum shell (i.e. water jacket or gel) along with heating element or heat/cool source.

If good ambient heat stability is available then a simplified system of a tightly controlled, rapid response heated stage may be all that is required. Low interior light levels for cell culture in an otherwise transparent carousel can be easily achieved by inserting opaque screens inside a small arc, and rotating the arc into the access port during non-viewing periods.

Turning to FIGS. 80-82, a micro-manipulator workstation 257 can be added to a linear well bank or carousel 261 of interchangeable sterile mini carousels 262, with access of the vertical micromanipulation tools through a notch cut into the jacket. Two or more workstations 258, 259 can be added around the perimeter of the carousel 261 or along the side (or opposite side) of a linear well bank to allow multiple operators to work simultaneously on several different wells. Each workstation can have its own micro-manipulation system, or can share a mobile micromanipulator mounted on a guide rail or swing arm 263. This allows movement of micro-tools and embryos or cell culture specimens or media across the carousel or positioned over other mini-carousels 260. The mini-carousels 260 mounted on the perimeter of a rotating master carousel 261 can be interchanged, removed, replaced, sterilized, or disposed of in a flexible system which also allows several operators to work at multiple workstations 257, 258, 259. For instance, an individualized mini-carousel 260 can be assigned to each patient in an IVF program, and the mini-carousel 260 can then be resterilized or disposed of after cycle completion.

One embodiment involves multiple swing arm micromanipulation workstations with 1, 2, or more micromanipulation tools available for sequential or for simultaneous use.

Micromanipulation tools are fixed or changeable, and can be manually or robotically maneuvered into and out of position. Programmable automated sequential positioning of tools allows rapid repetitive or intelligent micro-manipulation applications.

A large number of micromanipulation tools and instruments can be inserted into the x, y, and z-axis micro-actuator and made immediately available for a large number of cell culture, gamete, or embryo applications. Two or more micromanipulators can be loaded with fixed tools and used simultaneously or in rapid sequence within the same culture well, or multiple tools can be interchanged on micro-actuators as needed. Examples of some micro-tools are illustrated in FIG. 83 and include mechanical hatching needle 264, Tyrodes acid hatching pipette, micro-laser or microelectrode 265, ICSI insertion needle 266, blastomere biopsy needle 267, holding pipette 268, cell transfer pipette, embryo transfer catheter (end load) 269 or embryo transfer catheter (side load) 270, nylon loop 271, freezing pipette, thaw pipette, or oocyte stripping pipette, media sampler catheter.

A suggested prototype is illustrated in FIG. 84, consisting of nested carousels 272 and (in this example) two side view microscope work stations 273, 274. Microscope objectives have multiple magnification selections, and focus is by rack and pinion mount 275 on the workstation base 276. Two operators can review cultures or embryos and performed separate micromanipulation procedures simultaneously at stations 273 and 274. Small culture carousels 272 are interchangeable and replaceable through an incubator gate 277. Each carousel 272 can hold embryos, oocytes, and sperm for individual patients or couples, or each can hold embryos for specific developmental stages (i.e. cascading carousels, each assigned to a single post fertilization day). This allows loading carousels 272 on day 0 (egg capture) and leaving carousel 272 undisturbed inside incubator 278 until the day of embryo transfer or freezing on day 4 or 5, although embryos can still be periodically examined during this time at a workstation 273, 274. Individual automatic video photography can be done (for example once an hour to record a time lapse evaluation of embryo development for each embryo). Culture carousels 272 can be sterilized between use or can be disposable sterile items for single use or limited use, especially if culturing from patients with infectious agents (e.g. hepatitis B). Culture carousels 272 rotate into incubator 278 or workstation 273, 274 position on a master carousel 279. All carousels 272 are contained in an enclosed incubator 278 maintained at a constant controllable temperature. Separate overhead frames 280 support media tanks 281 and micromanipulators 282 to minimize vibration of micromanipulators 282. Media is supplied by tanks 281 containing control of dissolved gases and preheating elements, with flexible tubing 283 to feed media to fixed supply ring on the incubator, then on to plus and minus 180 degree ports on each culture carousel 272. Micromanipulators 282 are mounted on the overhead frame 280 or on swing arms 284, and are positioned directly over the working culture well 285 at each workstation 273, 274 when active.

FIG. 85 is a schematic of a full function microfluidic chip incorporating all of the basic functions described above. The full function chip contains an entry port 286, a retrieval port 287, fluid supply ports 288, fluid waste ports 289, micro-pumps 290 and micro-channels 291 controlled by microvalves 292 and a computer processing unit 293. Specimen incubation 294, staging 295, stripping, and coculture micro-chambers, along with detachable cassettes 296 and cryopreservation cassettes 297 are built into the chip design. Specimen and manipulation procedures are reviewed through a top view, side view, or inverted microscope, and temperature and ambient light are controlled by a standard or mini-incubator 298. Detachable cassettes 296 allow transfer of specimens between systems and individual control of cryopreservation 297 of specimens. Media is supplied by a dissolved gas cartridge 299 with filter to remove microorganisms and stray bubbles.

FIG. 86 is a two-tiered full function microfluidic system 302 incorporating entry, exit, and fluid supply ports along with sperm prep, oocyte prep and micromanipulation functions on the upper tier 300, and incubation in fluid trap stem microcassettes 303 on the lower tier 301, each detachable for cryopreservation.

A microfluidic system such as that described herein may be made using soft lithography plastic, Polymethylmethacrylate (PMMA), glass, DMSA or any material having similar qualities. One skilled in the art will understand the benefits and drawbacks of each of these materials.

I claim:

1-60. (canceled)

61. A freezing system comprising:
a microfluidic chip;
the chip having at least one port;
the chip having at least one microchamber;
at least one microchannel extending between the port and the microchamber.

62. The freezing system of claim 61 wherein the chip has multiple ports.

63. The freezing system of either claim 61 or 62 wherein the chip has multiple microchambers.

64. The freezing system of any of claims 61 to 63 wherein the chip has multiple microchannels.

65. The freezing system of any of claims 61 to 64 wherein the chip is thinner at the microchamber.

66. The freezing system of any of claims 61 to 65 wherein microchambers are located in the approximate center of the chip.

67. The freezing system of any of claims 61 to 66 wherein microchambers are located at the edges of the chip.

68. The freezing system of any of claims 61 to 67 having ribs between microchambers.

69. A method for freezing a specimen comprising the steps of:
immersing the specimen in culture fluid or fluid droplet;
placing the specimen in a chip having a stem;
positioning the specimen at the tip of the stem;
rapidly plunging the chip into a freezing agent; and
storing the chip at a temperature within a few degrees of absolute zero.

70. The method of claim 69 wherein the freezing agent is cryogen.

71. The method of either of claim 69 or 70 wherein the chip is plunged into the freezing agent stem first.

72. The method of claim 69 further comprising the step of inserting an inert gas bubble in the culture fluid.

73. The method of either claim 69 or claim 72 further comprising the step of adding cryoprotective solution.

74. The method of any of claims 69 to 73 further comprising the step of performing a cell culture on the specimen before freezing.

75. The method of any of claims 69 to 74 further comprising the step of thawing the specimen.

76. The method of claim 75 wherein the specimen is thawed by rapidly plunging the chip into warm water.

77. The method of either claim 75 or 76 wherein the specimen is thawed by exposure to radiant heat.

78. The method of any of claims 75 to 77 wherein the specimen is thawed by exposure to microwave.

79. The method of any of claims 73 to 78 further comprising the step of diluting the cryoprotective solution.

80. The method of any of claims 74 to 79 further comprising the step of diluting the cell culture of the specimen.

81. The method of any of claims 75 to 80 further comprising the step of retrieving the specimen from the stem.

82-152. (canceled)
